(19) United States
(12) Patent Application Publication
(10) Pub. No.: US 2012/0014870 A1
(43) Pub. Date: Jan. 19, 2012

(54) Diagnosis and Treatment of Cancer Using Anti-LGR7 Antibody

(75) Inventors: Hiroyuki Aburatani, Tokyo (JP); Yuriko Uehara, Tokyo (JP); Shinichi Funahashi, Tokyo (JP)

(73) Assignees: Forerunner Pharma Research Co., Ltd., Tokyo (JP); The University of Tokyo, Tokyo (JP)

(21) Appl. No.: 13/140,351

(22) PCT Filed: Dec. 25, 2009
(86) PCT No.: PCT/JP2009/071524

§ 371 Date: Oct. 5, 2011

(30) Foreign Application Priority Data
Dec. 26, 2008 (JP) 2008-333149

(51) Int. Cl. A61K 51/10; C40B 30/00; C07K 16/30; G01N 33/574

(52) U.S. Cl. 424/1.49; 436/501; 506/7; 530/389.7; 530/391.7; 530/387.3

(57) ABSTRACT

With dedicated research, the present inventors discovered that not only the LGR7 gene but also the LGR7 protein are highly expressed in clear cell adenocarcinoma cells of ovarian cancer. Furthermore, the present inventors found that anti-LGR7 antibodies have antibody-dependent cell-mediated cytotoxicity (ADCC) activity and complement-dependent cytotoxicity (CDC) activity against LGR7-expressing cells. From the above findings, the present inventors discovered that the anti-LGR7 antibodies are useful for diagnosis, prevention, and treatment of primary and metastatic ovarian clear cell adenocarcinoma.

### TECHNICAL FIELD

The present invention relates to antibodies that bind to the LGR7 protein, methods for diagnosing and treating cancer, and anticancer agents.

### BACKGROUND ART

The LGR7 molecule is a protein encoded by the gene of Ensembl ID ENSG00000171509 at 4q32 on human chromosome. Based on the features of its amino acid sequence, the molecule is classified as a member of the LGR family of the G-protein-coupled seven-transmembrane hormone receptor protein family (Leucine-rich GPCR family; hereinafter, referred to as LGR family) (Non-patent Document 1), and it is registered as NM_{—}021634/NP_{—}067647 in RefSeq. A sequence in which Leu at amino acid position 70 is substituted with Met has also been reported (Patent Document 1). Furthermore, three splice variants have been reported. In LGR7.1 (AY899848.1), exon 6a is inserted between exons 6 and 7, and exon 15a is inserted between exons 15 and 16. LGR7.2 (AY899849.1) has a gene structure from which exons 12 and 13 are deleted. In LGR7.10 (AY899850.1), exon 3 is deleted. Members of the LGR family are classified into three groups: the first group includes the hormone receptors FSHR(LGR1), LHCGR(LGR2), and TSHR(LGR3); the second group includes LGR4, LGR5, and LGR6 whose ligands are unknown; and the third group includes LGR7 and LGR8 whose ligands are relaxin, insulin-like peptide 3 (INSL3), and such (Non-patent Document 2). In all cases, the ligands are two heterogeneous peptides and they mainly transduce cAMP-mediated signals into cells. The LGR family has a structure comprising a seven-transmembrane protein region and a long N-terminal extracellular domain, and the extracellular domain has 9 to 17 leucine-rich repeats (LRRs) comprising about 25 amino acid residues. LGR7 has ten LRRs (Non-patent Document 1). It also has an LDL-A domain at the N terminus immediately before LRR, which is not found in other LGR family molecules (Non-patent Document 3). It has been reported that LDL-A is necessary for signal transduction, and it is involved in membrane trafficking of LGR7 (Non-patent Document 4). It is known from analyses of TSHR and such that in LGR family molecules, the extracellular LRR binds with high affinity to a ligand which further binds to the second extracellular loop domain, thereby causing G-protein-coupled signal transduction (Non-patent Document 5). Relaxins are known as ligands that bind to LGR7, and include human Relaxin 2 and Relaxin 3. Relaxin 2 has higher binding ability, and it is thought to function as a ligand of LGR7 in vivo (Non-patent Documents 6 and 7).

There is a report on the association of relaxin (ligand) with thyroid carcinoma and prostate cancer (Non-patent Document 8). Regarding prostate cancer, it is reported that androgen-independent growth is promoted in the prostate cancer cell line LNCaP introduced with p53 which has amino acid mutation R to H at position 273 (Non-patent Document 9). Since the level of H2 relaxin is elevated in these cells, it is suggested that H2 relaxin expression is involved in prostate cancer progression. It is shown that p53 R273H binds directly to the H2 relaxin promoter and induces PSA expression via an androgen receptor. However, there are still no articles that report the association between LGR7 and cancer.

On the other hand, some patent documents have reported higher LGR7 gene expression in uterine cancer and ovarian cancer than in normal tissues, and the association between LGR7 and cancer. However, it was not known whether LGR7-expressing cancers can be treated using antibodies, and none of the documents demonstrates an antibody-mediated anticancer effect (Patent Documents 2 to 5).

Even among ovarian cancers, clear cell adenocarcinoma is known to be a type of cancer that is less effective by chemotherapy (Non-patent Documents 10 and 11). Sugiyama et al. reported that the response rate towards chemotherapy using cisplatin and taxane compounds, which is a standard therapeutic method, is 72.5% in serous adenocarcinoma, and as low as 11.1% in clear cell adenocarcinoma (Non-patent Document 10). Meanwhile, the number of patients with clear cell adenocarcinoma is increasing in recent years. According to Nihon Sanfujinka Gakkai-shi Vol. 57, No. 11, p. 1711 (2005), clear cell adenocarcinoma accounts for 22% of all ovarian cancers in Japan. This is highly different from the number (6%) reported in the 1998 FIGO Annual Report overseas. Furthermore, according to the report by Japan Society of Obstetrics and Gynecology, the ratio of clear cell adenocarcinoma to all epithelial malignant tumors in ovarian cancers was 4% from 1971 to 1977, approximately 10% from 1978 to 1983, but more than 20% in 2002, and it is continuing to increase. Therefore, it is desirable to develop therapeutic methods against clear cell adenocarcinoma.

### PRIOR ART DOCUMENTS

### Patent Documents

[Patent Document 1] WO 9948921
[Patent Document 2] US 2005107595
[Patent Document 3] WO 2003016487
[Patent Document 4] WO 2003093827
[Patent Document 5] WO 2005107396

### [Non-Patent Documents]

[Non-Patent Document 1] Hsu, S. Y. et al., Molec. Endocr., 14, 1257-1271 (2000)
[Non-Patent Document 2] Hsueh A. J. W. et al., Journal of Endocrinology, 187, 333-338 (2005)
[Non-Patent Document 3] Bathgate R A. et al., Pharmacol Rev, 58, 7-31 (2006)
[Non-Patent Document 4] Kern A. et al., Endocrinology, 148, 1181-1194 (2007)
[Non-Patent Document 5] Kristiansen K., Pharmacology & Therapeutics, 103, 21-80 (2004)
[Non-Patent Document 6] Halls M. L. et al., British Journal of Pharmacology, 150, 677-691 (2007)
[Non-Patent Document 7] Van Der Westhuizen, E. T. et al., Current Drug Targets, 8, 91-104 (2007)
[Non-Patent Document 8] Hombach-Klonisch S. et al., American Journal of Pathology, 169, 617-632 (2006)
[Non-Patent Document 9] Vinall R. L. et al., Oncogene, 25, 2082-2093 (2006)
[Non-Patent Document 10] Sugiyama T et al. Cancer, 88, 2584 (2000)
[Non-Patent Document 11] Enomoto T st al. Proceedings of ASCO. 2003; 1797, Chicago

### SUMMARY OF THE INVENTION

### Problems to be Solved by the Invention

An objective of the present invention is to provide novel antibodies that bind to the LGR7 protein, novel methods for diagnosing cancer, novel methods for treating cancer, and novel cell growth inhibitors and anticancer agents.

### Means for Solving the Problems

The present inventors discovered that not only the LGR7 gene but also the LGR7 protein are highly expressed in clear cell adenocarcinoma cells of ovarian cancer. There has been no report that LGR7 is closely related to only one type of carcinoma among ovarian cancers—clear cell adenocarcinoma.

Furthermore, the present inventors produced monoclonal antibodies against the LGR7 protein.

The present inventors measured the antibody-dependent cell-mediated cytotoxicity (ADCC) activities of the anti-LGR7 antibodies, and discovered that the anti-LGR7 antibodies have ADCC activity against LGR7-expressing cells. The present inventors also measured the complement-dependent cell-mediated cytotoxicity (CDC) activities, and discovered that the anti-LGR7 antibodies have CDC activity against LGR7-expressing cells. In addition, the tumor regression effect of the anti-LGR7 antibodies was demonstrated by administering them to xenograft tumor model mice. Based on the above findings, the present inventors discovered that the anti-LGR7 antibodies are effective for diagnosis, prevention, and treatment of primary or metastatic ovarian clear cell adenocarcinoma, and thus completed the present invention. More specifically, the present inventors discovered that the anti-LGR7 antibodies are useful as tools for treatment and diagnosis of cancers highly expressing LGR7 such as ovarian clear cell adenocarcinoma, and thus completed the present invention.

Specifically, the present invention provides LGR7 protein-binding antibodies. Furthermore, the present invention provides LGR7 protein-binding antibodies that have cytotoxic activity against cells expressing the LGR7 protein. Preferably, the cytotoxic activity is ADCC activity. The present invention also provides anti-LGR7 antibodies bound with a cytotoxic substance.

Furthermore, the present invention provides pharmaceutical compositions comprising an LGR7 protein-binding antibody as an active ingredient. The present invention also provides cell growth inhibitors comprising an LGR7 protein-binding antibody as an active ingredient. The present invention provides anticancer agents comprising an LGR7 protein-binding antibody as an active ingredient.

The present invention also provides pharmaceutical compositions comprising an LGR7 protein-binding antibody and a pharmaceutically acceptable carrier. More specifically, the present invention provides the inventions of [1] to [23] below:
[1] An antibody that binds to an LGR7 protein and which has cell growth inhibitory activity against cells expressing the LGR7 protein.
[2] The antibody of [1], wherein the cell growth inhibitory activity is cytotoxic activity.
[3] The antibody of [2], wherein the cytotoxic activity is antibody-dependent cell-mediated cytotoxicity activity.
[4] The antibody of [2], wherein the cytotoxic activity is complement-dependent cytotoxicity activity.
[5] The antibody of any one of [1] to [4], which is an antibody to which a cytotoxic substance is bound.
[6] The antibody of [5], which is an antibody having an internalizing activity.
[7] The antibody of any one of [1] to [6], which is an antibody that suppresses the growth of cancer cells.
[8] The antibody of [7], wherein the cancer cells are clear cell ovarian cancer cells.
[9] An antibody of any one of (1) to (29) below:
   (1) an antibody comprising an H chain having the amino acid sequence of SEQ ID NO: 5 as CDR1, the amino acid sequence of SEQ ID NO: 6 as CDR2, and the amino acid sequence of SEQ ID NO: 7 as CDR3 (22DA6 heavy chain);
   (2) an antibody comprising an L chain having the amino acid sequence of SEQ ID NO: 10 as CDR1, the amino acid sequence of SEQ ID NO: 11 as CDR2, and the amino acid sequence of SEQ ID NO: 12 as CDR3 (22DA6 light chain);
   (3) an antibody comprising the H chain of (1) and the L chain of (2) (22DA6);
   (4) an antibody comprising an H chain having the amino acid sequence of SEQ ID NO: 15 as CDR1, the amino acid sequence of SEQ ID NO: 16 as CDR2, and the amino acid sequence of SEQ ID NO: 17 as CDR3 (22DA7 heavy chain);
   (5) an antibody comprising an L chain having the amino acid sequence of SEQ ID NO: 20 as CDR1, the amino acid sequence of SEQ ID NO: 21 as CDR2, and the amino acid sequence of SEQ ID NO: 22 as CDR3 (22DA7 light chain);
   (6) an antibody comprising the H chain of (4) and the L chain of (5) (22DA7);
   (7) an antibody comprising an H chain having the amino acid sequence of SEQ ID NO: 25 as CDR1, the amino acid sequence of SEQ ID NO: 26 as CDR2, and the amino acid sequence of SEQ ID NO: 27 as CDR3 (22DA17 heavy chain);
   (8) an antibody comprising an L chain having the amino acid sequence of SEQ ID NO: 30 as CDR1, the amino acid sequence of SEQ ID NO: 31 as CDR2, and the amino acid sequence of SEQ ID NO: 32 as CDR3 (22DA17 light chain);
   (9) an antibody comprising the H chain of (7) and the L chain of (8) (22DA17);
   (10) an antibody comprising an H chain having the amino acid sequence of SEQ ID NO: 35 as CDR1, the amino acid sequence of SEQ ID NO: 36 as CDR2, and the amino acid sequence of SEQ ID NO: 37 as CDR3 (22DA22 heavy chain);
   (11) an antibody comprising an L chain having the amino acid sequence of SEQ ID NO: 40 as CDR1, the amino acid sequence of SEQ ID NO: 41 as CDR2, and the amino acid sequence of SEQ ID NO: 42 as CDR3 (22DA22 light chain);
   (12) an antibody comprising the H chain of (10) and the L chain of (11) (22DA22);
   (13) an antibody comprising an H chain having the amino acid sequence of SEQ ID NO: 45 as CDR1, the amino acid sequence of SEQ ID NO: 46 as CDR2, and the amino acid sequence of SEQ ID NO: 47 as CDR3 (22DA23 heavy chain);
   (14) an antibody comprising an L chain having the amino acid sequence of SEQ ID NO: 50 as CDR1, the amino acid sequence of SEQ ID NO: 51 as CDR2, and the amino acid sequence of SEQ ID NO: 52 as CDR3 (22DA23 light chain);
   (15) an antibody comprising the H chain of (13) and the L chain of (14) (22DA23);
   (16) an antibody comprising an H chain having the amino acid sequence of SEQ ID NO: 55 as CDR1, the amino acid sequence of SEQ ID NO: 56 as CDR2, and the amino acid sequence of SEQ ID NO: 57 as CDR3 (22DA24 heavy chain);
   (17) an antibody comprising an L chain having the amino acid sequence of SEQ ID NO: 60 as CDR1, the amino acid sequence of SEQ ID NO: 61 as CDR2, and the amino acid sequence of SEQ ID NO: 62 as CDR3 (22DA24 light chain);
   (18) an antibody comprising the H chain of (16) and the L chain of (17) (22DA24);
   (19) an antibody comprising an H chain having the amino acid sequence of SEQ ID NO: 65 as CDR1, the amino acid sequence of SEQ ID NO: 66 as CDR2, and the amino acid sequence of SEQ ID NO: 67 as CDR3 (22SD7 heavy chain);
   (20) an antibody comprising an L chain having the amino acid sequence of SEQ ID NO: 70 as CDR1, the amino acid sequence of SEQ ID NO: 71 as CDR2, and the amino acid sequence of SEQ ID NO: 72 as CDR3 (22SD7 light chain);
   (21) an antibody comprising the H chain of (19) and the L chain of (20) (22SD7);
   (22) an antibody comprising an H chain having the amino acid sequence of SEQ ID NO: 75 as CDR1, the amino acid sequence of SEQ ID NO: 76 as CDR2, and the amino acid sequence of SEQ ID NO: 77 as CDR3 (22SD11 heavy chain);
   (23) an antibody comprising an L chain having the amino acid sequence of SEQ ID NO: 80 as CDR1, the amino acid sequence of SEQ ID NO: 81 as CDR2, and the amino acid sequence of SEQ ID NO: 82 as CDR3 (22SD11 light chain);
   (24) an antibody comprising the H chain of (22) and the L chain of (23) (22SD11);
   (25) an antibody comprising an H chain having the amino acid sequence of SEQ ID NO: 85 as CDR1, the amino acid sequence of SEQ ID NO: 86 as CDR2, and the amino acid sequence of SEQ ID NO: 87 as CDR3 (22SD48 heavy chain);
   (26) an antibody comprising an L chain having the amino acid sequence of SEQ ID NO: 90 as CDR1, the amino acid sequence of SEQ ID NO: 91 as CDR2, and the amino acid sequence of SEQ ID NO: 92 as CDR3 (22SD48 light chain);
   (27) an antibody comprising the H chain of (25) and the L chain of (26) (22SD48);
   (28) an antibody that has equivalent activity as the antibody of any one of (1) to (27);
   (29) an antibody that recognizes the same epitope recognized by the antibody of any one of (1) to (27).
[10] The antibody of [1] to [9], which has a human constant region.
[11] The antibody of [10], which is a chimeric antibody, humanized antibody, or human antibody.
[12] The antibody of any one of [1] to [11], which is a fucose-deficient antibody.
[13] A pharmaceutical composition comprising the antibody of any one of [1] to [12] as an active ingredient.
[14] A cell growth inhibitor comprising the antibody of any one of [1] to [12] as an active ingredient.
[15] An anticancer agent comprising the antibody of any one of [1] to [12] as an active ingredient.
[16] The anticancer agent of [15], wherein the cancer to be treated is ovarian cancer.
[17] The anticancer agent of [16], wherein the ovarian cancer is clear cell adenocarcinoma.
[18] A method for diagnosing cancer, which comprises detecting an LGR7 protein or a gene encoding an LGR7 protein.
[19] A method for diagnosing cancer, which comprises detecting an LGR7 protein.
[20] The diagnostic method of [19], wherein the LGR7 protein is detected using an antibody that binds to the LGR7 protein.
[21] A method for diagnosing cancer, which comprises the steps of:
   (a) providing a sample collected from a subject; and
   (b) detecting an LGR7 protein contained in the sample of (a) using an antibody that binds to the LGR7 protein.
[22] A method for diagnosing cancer, which comprises the steps of:
   (a) administering to a subject an antibody that has binding activity towards an LGR7 protein and which is labeled with a radioisotope; and
   (b) detecting accumulation of the radioisotope.
[23] The diagnostic method of any one of [18] to [22], wherein the cancer to be diagnosed is ovarian cancer.
[24] The diagnostic method of [23], wherein the ovarian cancer is clear cell adenocarcinoma.

### BRIEF DESCRIPTION OF THE DRAWINGS

FIG. 1-1 shows the expression profile of LGR7 (1552715_a_at) in ten cases of ovarian cancers including the following tissue types: clear cell carcinoma (four cases), serous adenocarcinoma (two cases), endometrioid adenocarcinoma (three cases), and carcinosarcoma (one case), as well as ten types of normal tissues, four types of fetal tissues, four types of ovarian cancer cell lines, and 87 ovarian cancer cases including three clear cell cancer cases from the U133 Plus 2.0 expression data published by the International Genomics Consortium (IGC). The letters indicated under the IGC sample names respectively refer to the following: C, clear cell adenocarcinoma; E, endometrioid adenocarcinoma; S, serous adenocarcinoma; M, mucinous adenocarcinoma; O, other cancers; and B, benign serous cystadenoma of borderline malignancy. JHOC-5, MCAS, RMG-1, RMUG-S, and TKY-nu are ovarian cancer cell lines, and among them, RMG-1 is a cell line derived from clear cell adenocarcinoma.

FIG. 1-2 shows the expression profile of LGR7 (1552715_a_at).

FIG. 1-3 shows the expression profile of LGR7 (1552715_a_at).

FIG. 2 presents a photograph showing the result of detecting LGR7 expression in HA-LGR7/BaF3#48 and HA-LGR7/DG44#24 by subjecting cell lysates to SDS-PAGE electrophoresis, and Western blotting using an anti-HA antibody (HA-7). Lane 1: BaF3; lane 2: HA-LGR7/BaF3#48; lane 3: DG44; and lane 4: HA-LGR7/DG44#24.

FIG. 3 shows the results of measuring ADCC activity by Cr release by using HA-LGR7/DG44 as the target cell and NK92mFcR3 as the effector cell.

FIG. 4 shows the results of measuring complement-dependent cytotoxicity by using HA-LGR7/DG44 as the target cell, and reacting it with an anti-LGR7 antibody together with a baby rabbit complement. The intensity of CDC activity was defined by the proportion of 7-AAD-incorporating cells to cells reacted with each antibody.

FIG. 5 shows the results of determining the number of viable cells by using HA-LGR7/DG44 as the target cell, and reacting it with Mab-ZAP (Saporin-labeled anti-mouse antibody) and each monoclonal antibody, and then performing WST8 assay. A higher value on the vertical axis indicates a larger number of viable cells.

FIG. 6 shows that 22DA17 and 22DA23 cross-react with mouse LGR7 based on the FACS analysis of HA-mLGR7/BaF3. The vertical axis indicates geometric mean values.

FIG. 7 shows the results of competition FACS analysis using the biotinylated antibodies Bio-22DA17 and Bio-22DA22. Whether antibodies recognize different epitopes was determined by observing the competition for the epitope by an unlabeled antibody. It was found that 22DA12 and 22DA22 are antibodies that recognize different epitopes from those of other antibodies. The solid lines indicate the results of reacting the biotinylated antibodies, and the shaded peaks indicate the results of reacting an FITC-recognizing anti-mouse antibody.

FIG. 8 shows the result of a drug efficacy test using a mouse xenograft model. In the graph, the tumor volume after antibody administration is plotted against the days post-tumor transplantation. The solid line shows the result of the PBS-administered group (negative control). The dashed line with squares shows the result of the group administered with 10 mg/kg of the FTKODA23 antibody, and the dashed line with triangles shows the result of the group administered with 2 mg/kg of the FTKODA23 antibody.

### MODE FOR CARRYING OUT THE INVENTION

### LGR7

In the present invention, LGR7 is a member protein of the seven-transmembrane LGR family. The amino acid sequence of human LGR7 and the gene sequence encoding it are disclosed in NCBI accession numbers NP_{—}067647 (SEQ ID NO: 1) and NM_{—}021634 (SEQ ID NO: 2), respectively. LGR7 used in the present invention may be splice variants or mutants. In the present invention, “LGR7 protein” refers to both the full-length protein and a fragment thereof. Herein, “fragment” refers to a polypeptide comprising any region of the LGR7 protein, and it does not necessarily have the function of the naturally-occurring LGR7 protein. An example of the fragment is a fragment comprising an extracellular domain of the LGR7 protein. The extracellular domains of the LGR7 protein correspond to positions 1 to 404, 462 to 485, 549 to 581, and 648 to 661 in the amino acid sequence of SEQ ID NO: 1. The transmembrane regions correspond to positions 405 to 427, 439 to 461, 486 to 508, 529-548, 582 to 604, 625-647, and 662 to 681 in the amino acid sequence of SEQ ID NO: 1.

### Preparation of the Anti-LGR7 Antibodies

The origin, type, form, and such of an anti-LGR7 antibody used in the present invention are not limited as long as it binds to the LGR7 protein. More specifically, known antibodies such as nonhuman animal antibodies (for example, mouse antibodies, rat antibodies, and camel antibodies), human antibodies, chimeric antibodies, humanized antibodies, and the like can be used. In the present invention, a monoclonal or polyclonal antibody can be used, but a monoclonal antibody is preferable. The binding between an antibody and the LGR7 protein is preferably specific. When an anti-LGR7 antibody used in the present invention is an antibody that recognizes human LGR7, it may specifically recognize human LGR7, or it may at the same time recognize LGR7 derived from another animal (for example, mouse LGR7).

An anti-LGR7 antibody used in the present invention can be obtained as either a polyclonal or monoclonal antibody using known means. The anti-LGR7 antibody used in the present invention is preferably a mammal-derived monoclonal antibody in particular. The mammal-derived monoclonal antibodies include antibodies produced by a hybridoma and antibodies produced by a host transformed with an expression vector containing an antibody gene using a genetic engineering method.

Basically, monoclonal antibody-producing hybridomas can be prepared using known techniques as follows. First, immunization is carried out by a conventional immunization method using the LGR7 protein as the sensitizing antigen. Hybridomas are prepared by fusing immune cells obtained from an immunized animal with a known parental cell using a conventional cell fusion method. Next, a hybridoma producing an anti-LGR7 antibody can be selected from the hybridomas by screening for a cell producing the antibody of interest using a conventional screening method.

Specifically, monoclonal antibodies are prepared, for example, as shown below. First, the LGR7 protein to be used as the sensitizing antigen for producing an antibody can be obtained by expressing the LGR7 gene. The nucleotide sequence of the LGR7 gene is disclosed in NCBI accession number NM_{—}021634 (SEQ ID NO: 2), etc. That is, a gene sequence encoding LGR7 is inserted into a known expression vector, and suitable host cells are transformed with this vector, and then the human LGR7 protein of interest can be purified from the host cells or culture supernatants using a known method. A purified naturally-occurring LGR7 protein can also be used. Furthermore, as utilized in the present invention, a fusion protein obtained by fusing a desired partial polypeptide of the LGR7 protein with a different polypeptide can also be used as the immunogen. For example, an antibody Fc fragment, peptide tag, or such can be used to produce a fusion protein as the immunogen. A vector expressing the fusion protein can be prepared by fusing genes encoding two or more desired polypeptide fragments in-frame, and inserting the fusion gene into an expression vector. Methods for preparing fusion proteins are described in Molecular Cloning, 2nd ed. (Sambrook J et al., Molecular Cloning 2nd ed., 9.47-9.58, Cold Spring Harbor Lab. Press, 1989).

An LGR7 protein thus purified can be used as a sensitizing antigen for use in immunizing a mammal. An LGR7 partial peptide can also be used as the sensitizing antigen. For example, the following peptides can be used as the sensitizing antigen:
a peptide obtained by chemical synthesis based on the amino acid sequence of human LGR7;
a peptide obtained by inserting a portion of the LGR7 gene into an expression vector and expressing it; and
a peptide obtained by protease degradation of the LGR7 protein.

There is no particular limitation on the region and size of LGR7 to be used as the partial peptide. A preferred region can be selected from the amino acid sequences constituting the extracellular domains of LGR7 (positions 1 to 404, 462 to 485, 549 to 581, and 648 to 661 in the amino acid sequence of SEQ ID NO: 1). The number of amino acids constituting a peptide to be used as the sensitizing antigen is preferably at least three or more, for example, five or more, or six or more. More specifically, a peptide having eight to 50 residues, preferably ten to 30 residues can be used as the sensitizing antigen.

There is no particular limitation on the mammal to be immunized with the sensitizing antigen. To obtain a monoclonal antibody by a cell fusion method, the animal to be immunized is preferably selected by considering the compatibility with the parental cell used for cell fusion. In general, rodents are preferable animals for immunization. More specifically, a mouse, rat, hamster, or rabbit can be used as the animal to be immunized. Alternatively, a monkey or such can be used as the animal to be immunized.

The above-mentioned animals can be immunized with the sensitizing antigen using a known method. In a general method, for example, a mammal can be immunized by intraperitoneal or subcutaneous injection of a sensitizing antigen. More specifically, the sensitizing antigen is administered to the mammal several times every four to 21 days. The sensitizing antigen is diluted at a suitable dilution ratio in phosphate-buffered saline (PBS), physiological saline, or such, and used for immunization. The sensitizing antigen can be administered together with an adjuvant. For example, the sensitizing antigen can be prepared by mixing and emulsifying with Freund's complete adjuvant. Furthermore, a suitable carrier can be used in immunization with the sensitizing antigen. In particular, when a partial peptide of low molecular weight is used as the sensitizing antigen, it is desirable to attach the sensitizing antigen peptide to a carrier protein such as albumin, keyhole limpet hemocyanin, and use this for immunization.

On the other hand, monoclonal antibodies can be obtained by DNA immunization. DNA immunization is a method that confers immune stimulation by administering into an immunized animal a vector DNA constructed in a form that allows an antigen protein-encoding gene (e.g., SEQ ID NO: 2) to be expressed in the immunized animal, and expressing the immunizing antigen in the body of the immunized animal. Compared to general immunization methods by which a protein antigen is administered, the following advantages can be expected from DNA immunization:
immune stimulation can be provided while retaining the structure of a membrane protein such as LGR7; and
there is no need to purify the immunizing antigen.

On the other hand, however, it is difficult to combine DNA immunization with an immune stimulatory means such as an adjuvant. It was predicted that since LGR7 has the structural feature of a seven-transmembrane conformation, it would be difficult to induce an immune response to LGR7 in vivo while retaining its naturally-occurring structure. Because of such structural feature, it was an unexpected result to actually have obtained by DNA immunization monoclonal antibodies that bind to LGR7, which is a protein belonging to the LGR family for which antibody production is difficult.

To obtain a monoclonal antibody of the present invention by DNA immunization, a DNA expressing the LGR7 protein is first administered to an animal to be immunized. The LGR7-encoding DNA can be synthesized by known methods such as PCR. The obtained DNA is inserted into a suitable expression vector and administered to the animal to be immunized. A commercially available expression vector such as pcDNA3.1 can be used as the expression vector. Methods generally used can be employed to administer the vector into the body. For example, DNA immunization can be carried out by using a gene gun to shoot gold particles adsorbed with the expression vector into cells.

A mammal is thus immunized, and an increase in the level of a desired antibody in the serum is observed. Then, immune cells are collected from the mammal and subjected to cell fusion. In particular, spleen cells can be preferably used as the immune cells.

A mammalian myeloma cell is used as a cell to be fused with the above-mentioned immunocyte. The myeloma cells preferably comprise a suitable selection marker for screening. A selection marker confers characteristics to cells for their survival (or failure to survive) under a specific culturing condition. Hypoxanthine-guanine phosphoribosyltransferase deficiency (hereinafter abbreviated as HGPRT deficiency), and thymidine kinase deficiency (hereinafter abbreviated as TK deficiency) are known as selection markers. Cells having HGPRT or TK deficiency have hypoxanthine-aminopterin-thymidine sensitivity (hereinafter abbreviated as HAT sensitivity). HAT-sensitive cells cannot carry out DNA synthesis in a HAT selection medium, and are thus killed. However, when the cells are fused with normal cells, they can continue to synthesize DNA using the salvage pathway of the normal cells, and therefore they can grow in the HAT selection medium.

HGPRT-deficient and TK-deficient cells can be selected in a medium containing 6-thioguanine or 8-azaguanine (hereinafter abbreviated as 8AG), and 5′-bromodeoxyuridine, respectively. Normal cells are killed since they incorporate these pyrimidine analogs into their DNA. On the other hand, cells that are deficient in these enzymes can survive in the selection medium, since they cannot incorporate these pyrimidine analogs. Alternatively, a selection marker referred to as G418 resistance provides resistance to 2-deoxystreptamine-type antibiotics (gentamycin analogs) from the neomycin-resistance gene. Various types of myeloma cells that are suitable for cell fusion are known. For example, myeloma cells including the following cells can be used to produce the monoclonal antibodies of the present invention:
P3 (P3x63Ag8.653) (J. Immunol. (1979) 123, 1548-1550);
P3x63Ag8U.1 (Current Topics in Microbiology and Immunology (1978) 81, 1-7);
NS-1 (Kohler. G and Milstein, C. Eur. J. Immunol. (1976) 6, 511-519);
MPC-11 (Margulies. D. H. et al., Cell (1976) 8, 405-415);
SP2/0 (Shulman, M. et al., Nature (1978) 276, 269-270);
FO (de St. Groth, S. F. et al., J. Immunol. Methods (1980) 35, 1-21);
S194 (Trowbridge, I. S. J. Exp. Med. (1978) 148, 313-323); and
R210 (Galfre, G et al., Nature (1979) 277, 131-133).

Cell fusion of the above-mentioned immunocytes with myeloma cells is essentially performed according to a known method, for example, the method of Kohler and Milstein et al. (Kohler. G and Milstein, C., Methods Enzymol. (1981) 73, 3-46).

More specifically, the above-mentioned cell fusion can be performed in a standard nutritional culture medium in the presence of, for example, a cell-fusion accelerator. A cell-fusion accelerator may be, for example, polyethylene glycol (PEG), Sendai virus (HVJ), or the like. If desired, an auxiliary agent such as dimethylsulfoxide can be added to further enhance fusion efficiency.

The ratio of immunocytes to myeloma cells used can be established at one's discretion. For example, the number of immunocytes is preferably set to one to ten times of that of myeloma cells. As a medium to be used for the above-mentioned cell fusion, for example, RPMI1640 medium and MEM medium, which are appropriate for the growth of the above-mentioned myeloma cell line, or other standard media that are used for this type of cell culture can be used. Moreover, a serum supplement solution such as fetal calf serum (FCS) can be added to the media.

Cell fusion is performed by thoroughly mixing predetermined amounts of the above-mentioned immunocytes and myeloma cells in the above-mentioned medium, adding and mixing with a PEG solution pre-heated to approximately 37° C., so as to form the desired fused cells (hybridomas). In the cell fusion method, for example, PEG with an average molecular weight of approximately 1000 to 6000 can generally be added at a concentration of 30 to 60% (w/v). Subsequently, the agent for cell fusion or the like which is unfavorable for the growth of hybridomas can be removed by successively adding an appropriate medium such as those listed above, removing the supernatant after centrifugation, and repeating these steps.

Hybridomas obtained in this manner can be selected using a selection medium appropriate for the selection markers carried by myelomas used for cell fusion. For example, cells that have HGPRT and TK deficiencies can be selected by culturing them in a HAT medium (a medium containing hypoxanthine, aminopterin, and thymidine). More specifically, when HAT-sensitive myeloma cells are used for cell fusion, cells that successfully fuse with normal cells can be selectively grown in the HAT medium. Culturing using the above-mentioned HAT medium is continued for a sufficient period of time to kill the cells other than the hybridoma of interest (non-fused cells). More specifically, the hybridoma of interest can be selected, typically by culturing for several days to several weeks. Subsequently, hybridomas that produce the antibody of interest can be screened and single isolated by carrying out a standard limiting dilution method. Alternatively, a LGR7-recognizing antibody can be prepared using the method described in International Patent Publication No. WO 03/104453.

Screening for an antibody of interest and cloning can be suitably carried out by a screening method based on known antigen-antibody reactions. For example, an antigen is bound to a carrier such as beads made of polystyrene or the like, or a commercially available 96-well microplate or such, and reacted with the culture supernatant of a hybridoma. Next, after rinsing away the carrier, a secondary antibody labeled with an enzyme, or such is reacted. If the antibody of interest that reacts with the sensitizing antigen is contained in the culture supernatant, the secondary antibody will bind to the carrier via the antibody. Finally, whether or not the antibody of interest is present in the culture supernatant can be determined by detecting the secondary antibody bound to the carrier. A hybridoma that produces a desired antibody having binding ability towards the antigen can be cloned using the limiting dilution method or such. Herein, as an antigen, the antigen used for immunization, or an LGR7 protein that is substantially identical thereto can be suitably used. For example, a cell line expressing LGR7, an extracellular domain of LGR7, or an oligopeptide that comprises a partial amino acid sequence constituting the domain can be used as the antigen.

Alternatively, instead of using the above method of obtaining the hybridoma by immunizing a non-human animal with an antigen, the antibody of interest can be obtained by sensitizing human lymphocytes with the antigen. More specifically, first, human lymphocytes are sensitized by the LGR7 protein in vitro. Then, the immunosensitized lymphocytes are fused with a suitable fusion partner. For example, human-derived myeloma cells that can divide indefinitely can be used as the fusion partner (see Japanese Patent Application Kokoku Publication No. (JP-B) H01-59878 (examined, approved Japanese patent application published for opposition)). The anti-LGR7 antibody obtained by this method is a human antibody having the activity to bind to the LGR7 protein.

Alternatively, an anti-LGR7 human antibody can be obtained by administering the LGR7 protein which serves as an antigen to a transgenic animal having the complete repertoire of human antibody genes, or by immunizing such an animal with a DNA constructed to express LGR7 in the animal. Antibody-producing cells from the immunized animal can be immortalized by cell fusion with a suitable fusion partner or treatment such as Epstein-Barr virus infection. A human antibody against the LGR7 protein can be isolated from the immortalized cells obtained in this manner (see International Publications WO94/25585, WO93/12227, WO92/03918, and WO94/02602). By further cloning the immortalized cells, it is possible to clone cells that produce an antibody having the reaction specificity of interest. When using a transgenic animal as the immunized animal, the immune system of the animal recognizes human LGR7 as a foreign substance. Thus, a human antibody against human LGR7 can be easily obtained.

The monoclonal antibody-producing hybridomas produced in this manner can be passaged and cultured in a standard medium. Alternatively, the hybridomas can be stored for a long period in liquid nitrogen.

The hybridomas can be cultured according to a standard method, and the monoclonal antibody of interest can be obtained from the culture supernatants. Alternatively, the hybridomas can be grown by administering them to a compatible mammal, and monoclonal antibodies can be obtained as its ascites. The former method is suitable for obtaining highly purified antibodies.

In the present invention, an antibody encoded by an antibody gene cloned from antibody-producing cells can be used. The cloned antibody gene can be incorporated into a suitable vector and then introduced into a host to express the antibody. Methods for isolating an antibody gene, introducing the gene into a vector, and transforming host cells have been established (see for example, Vandamme, A. M. et al., Fur. J. Biochem. (1990) 192, 767-775).

For example, a cDNA encoding the variable region (V region) of an anti-LGR7 antibody can be obtained from hybridoma cells producing the anti-LGR7 antibody. Usually, in order to accomplish this, first, total RNA is extracted from the hybridoma. For example, the following methods can be used as methods for extracting mRNA from cells:
the guanidine ultracentrifugation method (Chirgwin, J. M. et al., Biochemistry (1979) 18, 5294-5299); and
the AGPC method (Chomczynski, P. et al., Anal. Biochem. (1987) 162, 156-159).

The extracted mRNA can be purified using an mRNA purification kit (manufactured by GE Healthcare Biosciences) or the like. Alternatively, kits such as the QuickPrep mRNA Purification Kit (manufactured by GE Healthcare Biosciences) for direct extraction of total mRNA from cells are commercially available. Total mRNA can be obtained from a hybridoma using such a kit. A cDNA encoding the antibody V region can be synthesized from the obtained mRNA using a reverse transcriptase. An arbitrary sequence of 15 to 30 bases selected from a sequence common to the mouse antibody genes can be used as a primer. Specifically, a cDNA encoding the antibody V region can be obtained using primers having the DNA sequences of SEQ ID NOs: 97 to 100. A cDNA can be synthesized using the AMV Reverse Transcriptase First-strand cDNA Synthesis Kit (manufactured by Seikagaku Corporation), etc. Alternatively, the 5′-Ampli FINDER RACE Kit (manufactured by Clontech) and the 5′-RACE method which utilizes PCR (Frohman, M. A. et al., Proc. Natl. Acad. Sci. USA (1988) 85, 8998-9002; Belyaysky, A. et al., Nucleic Acids Res. (1989) 17, 2919-2932) can be used for synthesizing and amplifying cDNA. Additionally, during the process of cDNA synthesis, suitable restriction enzyme sites mentioned below can be introduced into both ends of the cDNA.

The cDNA fragment of interest is purified from the obtained PCR product and then ligated to a vector DNA. The recombinant vector is thus prepared, and introduced into Escherichia coli (E. coli) and the like, and colonies are selected. Then, a desired recombinant vector can be prepared from the colony-forming E. coli. The nucleotide sequence of the cDNA can be verified by a known method such as the dideoxynucleotide chain termination method.

Furthermore, a cDNA library can be used to obtain a gene encoding an antibody variable region. First, cDNAs are synthesized using mRNAs extracted from antibody-producing cells as a template to obtain a cDNA library. A commercially available kit is conveniently used for synthesis of the cDNA library. In practice, the quantity of mRNAs obtained from only few cells is very small; thus, the yield is low if the mRNAs are purified directly. Therefore, purification is usually carried out after addition of a carrier RNA which clearly does not contain an antibody gene. Alternatively, when a certain quantity of RNAs can be extracted, the RNAs of antibody-producing cells themselves can be efficiently extracted. For example, addition of carrier RNA is not necessary in some cases when RNAs are extracted from 10 or more, or 30 or more, or preferably 50 or more antibody-producing cells.

The antibody gene is amplified by the PCR method using the obtained cDNA library as a template. Primers for amplifying an antibody gene by the PCR method are known. For example, it is possible to design primers for amplifying a human antibody gene based on the disclosure of the article (J. Mol. Biol. (1991), 222, 581-597), and the like. These primers have different nucleotide sequences depending on the immunoglobulin subclass. Therefore, all possibilities should be taken into consideration when the PCR method is carried out using a cDNA library of unknown subclass as a template.

Specifically, for example, when the objective is to obtain a gene encoding human IgG one can use primers capable of amplifying genes encoding γ1 to γ5 as the heavy chain, and κ and λ chains as the light chain. To amplify an IgG variable region gene, a primer that anneals to a portion corresponding to the hinge region is generally used as the 3′-end primer. Meanwhile, a primer for the individual subclass can be used as the 5′-end primer.

The PCR products produced by gene amplification primers for each heavy- or light-chain subclass are made into independent libraries. Using the libraries thus synthesized, immunoglobulins comprising a combination of heavy and light chains can be reconstructed. The antibody of interest can be screened using the binding activity of a reconstructed immunoglobulin toward LGR7 as an indicator.

For example, when the objective is to obtain an antibody against LGR7, it is more preferable that the binding between the antibody and LGR7 is specific. An LGR7-binding antibody may be screened, for example, by the following steps:
(1) contacting LGR7 with an antibody comprising a V region encoded by an obtained cDNA;
(2) detecting the binding between LGR7 and the antibody; and
(3) selecting an antibody that binds to LGR7.

A method for detecting the binding between an antibody and LGR7 is known. More specifically, a test antibody is reacted with LGR7 immobilized onto a carrier, and then a labeled antibody that recognizes the antibody is reacted. If the labeled antibody on the carrier is detected after washing, then the binding between the test antibody and LGR7 can be verified. Enzymatically active proteins such as peroxidase and β-galactosidase, or fluorescent substances such as FITC can be used for the label. A fixed specimen of LGR7-expressing cells can be used to evaluate the binding activity of the antibody.

Alternatively, for an antibody screening method based on the binding activity, a phage vector-based panning method may be used. When the antibody genes are obtained as libraries of the heavy-chain and light-chain subclasses from polyclonal antibody-expressing cells as mentioned above, phage displaying methods are advantageous. Genes encoding variable regions of the heavy and light chains can be made into a single-chain Fv (scFv) gene by linking the genes via suitable linker sequences. Phages expressing an scFv on their surface can be obtained by inserting a gene encoding the scFv into a phagemid vector. DNA encoding an scFv having the binding activity of interest can be collected by contacting the phage with an antigen of interest, and then collecting antigen-bound phage. scFv having the binding activity of interest can be enriched by repeating this operation as necessary.

An antibody-encoding polynucleotide of the present invention may encode a full-length antibody or a portion of the antibody. “A portion of an antibody” refers to any portion of an antibody molecule. Hereinafter, the term “antibody fragment” may be used to refer to a portion of an antibody. A preferred antibody fragment of the present invention comprises the complementarity determination region (CDR) of an antibody. More preferably, an antibody fragment of the present invention comprises all of the three CDRs that constitute a variable region.

Once a cDNA encoding the V region of an anti-LGR7 antibody of interest is obtained, this cDNA is digested with restriction enzymes that recognize the restriction enzyme sites inserted to both ends of the cDNA. A preferred restriction enzyme recognizes and digests a nucleotide sequence that is less likely to appear in the nucleotide sequence constituting the antibody gene. Furthermore, to insert a single copy of the digested fragment into a vector in the correct direction, a restriction enzyme that provides sticky ends is preferable. A cDNA encoding the anti-LGR7 antibody V region, which has been digested as described above, is inserted into a suitable expression vector to obtain the antibody expression vector. In this step, a chimeric antibody can be obtained by fusing a gene encoding the antibody constant region (C region) with the above-mentioned gene encoding the V region in frame. Herein, “chimeric antibody” refers to an antibody whose constant and variable regions are derived from different origins. Therefore, in addition to interspecies chimeric antibodies such as mouse-human chimeric antibodies, human-human intraspecies chimeric antibodies are also included in the chimeric antibodies of the present invention. A chimeric antibody expression vector can also be constructed by inserting the V-region gene into an expression vector into which a constant region-encoding gene has been introduced.

More specifically, for example, the restriction enzyme recognition sequence for a restriction enzyme that digests the V-region gene can be placed at the 5′ end of a DNA encoding a desired antibody constant region (C region) in an expression vector. The chimeric antibody expression vector is constructed by digesting the two vectors using the same combination of restriction enzymes, and fusing them in frame.

To produce an anti-LGR7 antibody used in the present invention, the antibody gene can be incorporated into an expression vector so that it is expressed under the regulation of an expression control region. The expression regulatory region for antibody expression includes, for example, an enhancer or a promoter. Then, by transforming suitable host cells with this expression vector, recombinant cells that carry the DNA expressing the anti-LGR7 antibody can be obtained.

To express an antibody gene, a DNA encoding the antibody heavy chain (H-chain) and a DNA encoding the antibody light chain (L-chain) can be incorporated separately into expression vectors. An antibody molecule comprising the H-chain and L-chain can be expressed by simultaneously transfecting (co-transfecting) the H-chain and L-chain-incorporated vectors into the same host cell. Alternatively, DNAs encoding the H-chain and L-chain can be incorporated into a single expression vector to transform a host cell with the vector (see International Patent Publication No. WO 94/11523).

Many combinations of hosts and expression vectors for introducing an isolated antibody gene into an appropriate host to produce the antibody are known. Any of these expression systems can be applied to the present invention. When using eukaryotic cells as a host, animal cells, plant cells, and fungal cells can be used. More specifically, animal cells that may be used in the present invention are, for example, the following cells:
(1) mammalian cells such as CHO, COS, myeloma, baby hamster kidney (BHK), HeLa, Vero, HEK293, Ba/F3, HL-60, Jurkat, and SK-HEP1 cells;
(2) amphibian cells such as Xenopus oocytes; and
(3) insect cells such as sf9, sf21, Tn5.

In addition, as a plant cell system, an antibody gene expression system using cells derived from the Nicotiana genus such as Nicotiana tabacum is known. Callus-cultured cells can be used to transform plant cells.

Furthermore, the following cells can be used as fungal cells; yeasts: the Saccharomyces genus, for example, Saccharomyces cerevisiae, and the Pichia genus, for example, Pichia pastoris; and filamentous fungi: the Aspergillus genus, for example, Aspergillus niger.

Antibody gene expression systems that utilize prokaryotic cells are also known. For example, when using bacterial cells, E. coli cells, Bacillus subtilis cells, and such may be used in the present invention.

For mammalian cells, the antibody genes can be expressed by operatively placing the antibody gene just behind a commonly used effective promoter, and a polyA signal on the 3′ downstream side of the antibody gene. An example of such promoter/enhancer is human cytomegalovirus immediate early promoter/enhancer.

Other promoters/enhancers that can be used for antibody expression include viral promoters/enhancers, or mammalian cell-derived promoters/enhancers such as human elongation factor 1α (HEF1α). Specific examples of viruses whose promoters/enhancers may be used include retrovirus, polyoma virus, adenovirus, and simian virus 40 (SV40).

When an SV40 promoter/enhancer is used, the method of Mulligan et al. (Nature (1979) 277, 108) may be utilized. An HEF1a promoter/enhancer can be readily used for expressing a gene of interest by the method of Mizushima et al. (Nucleic Acids Res. (1990) 18, 5322).

In the case of E. coli, the antibody can be expressed by operatively placing the antibody gene with a signal sequence for secretion at downstream of a commonly used effective promoter Examples of such promoter include the lacZ promoter and araB promoter. For the lacZ promoter, the method of Ward et al., (Nature (1989) 341, 544-546; FASEB J. (1992) 6, 2422-2427) may be used. Alternatively, the araB promoter can be used for expressing a gene of interest by the method of Better et al. (Science (1988) 240, 1041-1043).

The pelB signal sequence for secretion (Lei, S. P. et al., J. Bacteriol. (1987) 169, 4379) may be used for antibody production in the periplasm of E. coli. After isolation of the antibody produced in the periplasm, the antibody can be refolded by using a protein denaturant like guanidine hydrochloride or urea so that the antibody will have the desired binding activity.

When the antibody is produced using animal cells, it is desirable to use a signal sequence of an antibody heavy- or light-chain gene as the signal sequence necessary for secretion to the outside of the cells. Alternatively, the signal sequences possessed by secretory proteins such as IL-3 and IL-6 can be used.

A replication origin derived from SV40, a polyomavirus, adenovirus, bovine papilloma virus (BPV), or such can be used and inserted into an expression vector. Additionally, a selection marker can be inserted into the expression vector for increasing the gene copy number in the host cell system. More specifically, the following selection markers can be used: aminoglycoside transferase (APH) gene;
thymidine kinase (TK) gene;
E. coli xanthine guanine phosphoribosyl transferase (Ecogpt) gene; and
dihydrofolate reductase (dhfr) gene, etc.

The antibody of interest is produced by introducing these expression vectors into host cells, and then culturing the transformed host cells either in vitro or in vivo. The host cells are cultured according to known methods. For example, DMEM, MEM, RPMI 1640, or IMDM can be used as the liquid culture medium, and a serum-based supplement such as fetal calf serum (FCS) can be used in combination.

The antibody expressed and produced as described above can be purified by using known methods conventionally used for protein purification either singly or in a suitable combination. For example, the antibody can be separated and purified by suitably selecting and combining an affinity column such as a protein A column, a chromatography column, a filter, ultrafiltration, salting-out, dialysis, and such (Antibodies: A Laboratory Manual. Ed Harlow, David Lane, Cold Spring Harbor Laboratory, 1988).

In addition to the above host cells, transgenic animals can also be used to produce a recombinant antibody. That is, the antibody can be obtained from an animal into which the gene encoding the antibody of interest is introduced. For example, the antibody gene can be inserted in frame into a gene that encodes a protein produced inherently in milk to construct a fused gene. Goat β-casein or such can be used, for example, as the protein secreted in milk. A DNA fragment containing the fused gene inserted with the antibody gene is injected into a goat embryo, and then this embryo is introduced into a female goat. Desired antibodies can be obtained as a protein fused with the milk protein from milk produced by the transgenic goat born from the goat that received the embryo (or progeny thereof). To increase the volume of milk containing the desired antibody produced by the transgenic goat, hormones can be used on the transgenic goat as necessary (Ebert, K. M. et al., Bio/Technology (1994) 12, 699-702).

Non-human animal-derived antibody C regions can be used for the C regions of a recombinant antibody of the present invention. For example, Cγ1, Cγ2a, Cγ2b, Cγ3, Cμ, Cδ, Cα1, Cα2, and Cc can be used for the mouse antibody H-chain C-region, and Cκ and Cλ can be used for the L-chain C-region. In addition, antibodies of animals other than mice such as rats, rabbits, goat, sheep, camels, and monkeys can be used. Their sequences are known. Furthermore, the C region can be modified to improve the stability of the antibodies or their production.

In the present invention, when administering antibodies to humans, genetically recombinant antibodies that have been artificially modified for the purpose of reducing xenoantigenicity against humans, or the like can be used. Examples of the genetically recombinant antibodies include chimeric antibodies and humanized antibodies. These modified antibodies can be produced using known methods.

A chimeric antibody is an antibody whose variable regions and constant regions are of different origins. For example, an antibody comprising the heavy-chain and light-chain variable regions of a mouse antibody and the heavy-chain and light-chain constant regions of a human antibody is a mouse-human interspecies chimeric antibody. A recombinant vector expressing a chimeric antibody can be produced by ligating a DNA encoding a mouse antibody variable region to a DNA encoding a human antibody constant region, and then inserting it into an expression vector. The recombinant cells that have been transformed with the vector are cultured, and the incorporated DNA is expressed to obtain the chimeric antibody produced in the culture. Human C regions are used for the C regions of chimeric antibodies and humanized antibodies.

For example, Cγ1, Cγ2, Cγ3, Cγ4, Cμ, Cα1, Cα2, and Cε can be used as an H-chain C region. Cκ and Cλ can be used as an L-chain C region. The amino acid sequences of these C regions and the nucleotide sequences encoding them are known. Furthermore, the human antibody C region can be modified to improve the stability of an antibody or its production.

Generally, a chimeric antibody consists of the V region of an antibody derived from a non-human animal, and a C region derived from a human antibody. On the other hand, a humanized antibody consists of the complementarity determining region (CDR) of an antibody derived from a non-human animal, and the framework region (FR) and C region derived from a human antibody. Since the antigenicity of a humanized antibody in human body is reduced, a humanized antibody is useful as an active ingredient for therapeutic agents of the present invention.

The antibody variable region generally comprises three complementarity-determining regions (CDRs) separated by four framework regions (FRs). CDR is a region that substantially determines the binding specificity of an antibody. The amino acid sequences of CDRs are highly diverse. On the other hand, the FR-constituting amino acid sequences are often highly homologous even among antibodies with different binding specificities. Therefore, generally, it is said that the binding specificity of a certain antibody can be transferred to another antibody by CDR grafting.

A humanized antibody is also called a reshaped human antibody. Specifically, humanized antibodies prepared by grafting the CDR of a non-human animal antibody such as a mouse antibody to a human antibody and such are known. Common genetic engineering technologies for obtaining humanized antibodies are also known.

Specifically, for example, overlap extension PCR is known as a method for grafting a mouse antibody CDR to a human FR. In overlap extension PCR, a nucleotide sequence encoding a mouse antibody CDR to be grafted is added to the primers for synthesizing a human antibody FR. Primers are prepared for each of the four FRs. It is generally said that when grafting a mouse CDR to a human FR, selecting a human FR that is highly homologous to a mouse FR is advantageous for maintaining the CDR function. That is, it is generally preferable to use a human FR comprising an amino acid sequence highly homologous to the amino acid sequence of the FR adjacent to the mouse CDR to be grafted.

Nucleotide sequences to be ligated are designed so that they will be connected to each other in frame. Human FRs are individually synthesized using the respective primers. As a result, products in which the mouse CDR-encoding DNA is attached to the individual FR-encoding DNAs are obtained. Nucleotide sequences encoding the mouse CDR of each product are designed so that they overlap with each other. Then, overlapping CDR regions of the products synthesized using a human antibody gene as the template are annealed for complementary strand synthesis reaction. By this reaction, human FRs are ligated through the mouse CDR sequences.

The full length of the V-region gene, in which three CDRs and four FRs are ultimately ligated, is amplified using primers that anneal to its 5′ and 3′ ends and which have suitable restriction enzyme recognition sequences. A vector for human antibody expression can be produced by inserting the DNA obtained as described above and a DNA that encodes a human antibody C region into an expression vector so that they will ligate in frame. After transfecting this vector into a host to establish recombinant cells, the recombinant cells are cultured, and the DNA encoding the humanized antibody is expressed to produce the humanized antibody in the culture of the cells (see, European Patent Publication No. EP 239,400, and International Patent Publication No, WO 96/0257).

By qualitatively or quantitatively measuring and evaluating the antigen-binding activity of the humanized antibody produced as described above, one can suitably select human antibody FRs that allow CDRs to form a favorable antigen-binding site when ligated through the CDRs. As necessary, amino acid residues in an FR may be substituted so that the CDRs of a reshaped human antibody form an appropriate antigen-binding site. For example, amino acid sequence mutations can be introduced into FRs by applying the PCR method used for fusing a mouse CDR with a human FR. More specifically, partial nucleotide sequence mutations can be introduced into primers that anneal to the FR sequence. Nucleotide sequence mutations are introduced into the FRs synthesized using such primers. Mutant FR sequences having the desired characteristics can be selected by measuring and evaluating the activity of the amino acid-substituted mutant antibody to bind to the antigen by the above-mentioned method (Sato, K. et al., Cancer Res. 1993, 53, 851-856).

Methods for obtaining human antibodies are also known. For example, human lymphocytes are sensitized in vitro with a desired antigen or cells expressing a desired antigen. Next, the desired human antibody which has the activity to bind to the antigen can be obtained by fusing a sensitized lymphocyte with a human myeloma cell (see JP-B H01-59878). For example, U266 and the like can be used as the human myeloma cell which serves as the fusion partner.

In addition, a desired human antibody can be obtained by immunizing a transgenic animal having the complete repertoire of human antibody genes with a desired antigen. (see International Publication Nos, WO 93/12227, WO 92/03918, WO 94/02602, WO 94/25585, WO 96/34096, and WO 96/337351. Furthermore, technologies for obtaining a human antibody by panning using a human antibody library are known. For example, a human antibody V region can be expressed on the surface of a phage as a single-chain antibody (scFv) by the phage display method, and a phage that binds to the antigen can be selected. The DNA sequence encoding the V region of the human antibody that binds to the antigen can be determined by analyzing the genes of the selected phage. After the DNA sequence of the scFv that binds to the antigen is determined, an expression vector can be prepared by fusing the V-region sequence in-frame with the sequence of a desired human antibody C region, and then inserting this into a suitable expression vector. The expression vector is introduced into suitable expression cells such as those described above, and the human antibody can be obtained by expressing the human antibody-encoding gene. These methods are already known (International Publication Nos. WO 92/01047, WO 92/20791, WO 93/06213, WO 93/11236, WO 93/19172, WO 95/01438, and WO 95/15388).

Therefore, in a preferred embodiment, the antibodies used in the present invention include an antibody comprising a human constant region.

The antibodies of the present invention are not limited to bivalent antibodies represented by IgG, but include monovalent antibodies and multivalent antibodies represented by IgM, as long as it binds to the LGR7 protein. The multivalent antibody of the present invention includes a multivalent antibody that has the same antigen binding sites, and a multivalent antibody that has partially or completely different antigen binding sites. The antibody of the present invention is not limited to the whole antibody molecule, but includes low-molecular-weight antibodies (minibodies) and modified products thereof, as long as they bind to the LGR7 protein.

A low-molecular-weight antibody contains an antibody fragment lacking a portion of a whole antibody (for example, whole IgG). As long as it has the ability to bind the LGR7 antigen, partial deletions of an antibody molecule are permissible. Antibody fragments of the present invention preferably contain a heavy-chain variable region (VH) and/or a light-chain variable region (VL). Furthermore, antibody fragments of the present invention preferably contain CDR. The amino acid sequence of VH or VL may have substitutions, deletions, additions, and/or insertions. Furthermore, as long as it has the ability to bind the LGR7 antigen, VH and/or VL can be partially deleted. The variable region may be chimerized or humanized. Specific examples of the antibody fragments include Fab, Fab′, F(ab′)2, and Fv. Specific examples of low-molecular-weight antibodies include Fab, Fab′, F(ab′)2, Fv, scFv (single chain Fv), diabody, sc(Fv)2 (single chain (Fv)2), and scFv-Fc. Multimers of these antibodies (for example, dimers, trimers, tetramers, and polymers) are also included in the low-molecular-weight antibodies of the present invention.

Fragments of antibodies can be obtained by treating an antibody with an enzyme to produce antibody fragments. Known enzymes that produce antibody fragments are, for example, papain, pepsin, and plasmin. Alternatively, genes encoding these antibody fragments can be constructed, introduced into expression vectors, and then expressed in appropriate host cells (see, for example, Co, M. S. et al., J. Immunol. (1994) 152, 2968-2976; Better, M. and Horwitz, A. H., Methods in Enzymology (1989) 178, 476-496; Plueckthun, A. and Skerra, A., Methods in Enzymology (1989) 178, 476-496; Lamoyi, E., Methods in Enzymology (1989) 121, 652-663; Rousseaux, J. et al., Methods in Enzymology (1989) 121, 663-669; and Bird, R. E. et al., TIBTECH (1991) 9, 132-137).

Digestive enzymes cleave specific sites of an antibody fragment, and yield antibody fragments with the following specific structures. When genetic engineering technologies are used on such enzymatically obtained antibody fragments, any portion of the antibody can be deleted.
Papain digestion: F(ab)2 or Fab
Pepsin digestion: F(ab′)2 or Fab′
Plasmin digestion: Facb

Therefore, low-molecular-weight antibodies of the present invention may be antibody fragments lacking any region, as long as they have binding affinity to LGR7. Furthermore, according to the present invention, the antibodies desirably maintain their effector activity, particularly in the treatment of cell proliferative diseases such as cancer. More specifically, preferred low-molecular-weight antibodies of the present invention have both binding affinity to LGR7 and effector function. The antibody effector function includes ADCC activity and CDC activity. Particularly preferably, therapeutic antibodies of the present invention have ADCC activity and/or CDC activity as effector function.

A diabody refers to a bivalent antibody fragment constructed by gene fusion (Hollinger P. et al., Proc. Natl. Acad. Sci. USA 90: 6444-6448 (1993); EP 404,097; WO 93/11161; and such). A diabody is a dimer composed of two polypeptide chains. Generally, in each polypeptide chain constituting the dimer, VL and VH are linked by a linker within the same chain. The linker in a diabody is generally short enough to prevent binding between VL and VH. Specifically, the amino acid residues constituting the linker are, for example, five residues or so. Therefore, VL and VH that are encoded by the same polypeptide chain cannot form a single-chain variable region fragment, and form a dimer with another single chain variable region fragment. As a result, diabodies have two antigen binding sites.

scFv can be obtained by ligating the H-chain V region and L-chain V region of an antibody. In scFv, the H-chain V region and L-chain V region are ligated via a linker, preferably a peptide linker (Huston, J. S. et al., Proc. Natl. Acad. Sci. U.S.A., 1988, 85, 5879-5883). The H-chain V region and L-chain V region of scFv may be derived from any of the antibodies described herein. The peptide linker for ligating the V regions is not particularly limited. For example, any single-chain peptide consisting of 3 to 25 residues or so can be used as the linker. More specifically, for example, peptide linkers described below or such can be used.

PCR methods such as those described above can be used for ligating the V regions of both chains. For ligation of the V regions by PCR methods, first, a whole DNA or a DNA encoding a desired partial amino acid sequence selected from the following DNAs can be used as a template:
a DNA sequence encoding the H chain or the H-chain V region of the antibody; and
a DNA sequence encoding the L chain or the L-chain V region of the antibody.

DNAs encoding the H-chain and L-chain V regions are individually amplified by PCR methods using a pair of primers that have sequences corresponding to the sequences of both ends of the DNA to be amplified. Then, a DNA encoding the peptide linker portion is prepared. The DNA encoding the peptide linker can also be synthesized using PCR. To the 5′ end of the primers used, nucleotide sequences that can be ligated to each of the individually synthesized V-region amplification products are added. Then, PCR reaction is carried out using the “H-chain V region DNA”, “peptide linker DNA”, and “L-chain V region DNA”, and the primers for assembly PCR.

The primers for assembly PCR consist of the combination of a primer that anneals to the 5′ end of the “H-chain V region DNA” and a primer that anneals to the 3′ end of the “L-chain V region DNA”. That is, the primers for assembly PCR are a primer set that can amplify a DNA encoding the full-length sequence of scFv to be synthesized. On the other hand, nucleotide sequences that can be ligated to each V-region DNA are added to the “peptide linker DNA”. Thus, these DNAs are ligated, and the full-length scFv is ultimately produced as an amplification product using the primers for assembly PCR. Once the scFv-encoding DNA is constructed, expression vectors containing the DNA, and recombinant cells transformed by these expression vectors can be obtained according to conventional methods. Furthermore, the scFvs can be obtained by culturing the resulting recombinant cells and expressing the scFv-encoding DNA.

scFv-Fc is a low-molecular-weight antibody produced by fusing an Fc domain to an scFV comprising the H-chain V region and L-chain V region of an antibody (Cellular & Molecular Immunology 2006; 3: 439-443). While there is no particular limitation on the origin of the scFv used for scFv-Fc, for example, scFv derived from IgM may be used. Furthermore, while there is no particular limitation on the origin of Fc, for example, mouse IgG (mouse IgG2a and such) and human IgG (human IgG1 and such) may be used. Therefore, in a preferred embodiment, examples of scFv-Fc include an scFv-Fc produced by linking the scFv fragment of an IgM antibody to CH2 (for example, Cγ2) and CH3 (for example, Cγ3) of mouse IgG2a with the hinge region (Hγ) of mouse IgG2a, and an scFv-Fc produced by linking the scFv fragment of an IgM antibody to CH2 and CH3 of human IgG1 with the hinge region of human IgG1.

sc(Fv)2 is a minibody prepared by ligating two VHs and two VLs with linkers or such to form a single chain (Hudson et al., J. Immunol. Methods 1999; 231: 177-189). sc(Fv)2 can be produced, for example, by joining scFvs with a linker.

Moreover, antibodies in which two VHs and two VLs are arranged in the order of VH, VL, VH, and VL ([VH]-linker-[VL]-linker-[VH]linker-[VL]), starting from the N-terminal side of a single chain polypeptide, are preferred.

The order of the two VHs and the two VLs is not particularly limited to the above-mentioned arrangement, and they may be placed in any order. Examples include the following arrangements:
[VL]-linker-[VH]-linker-[VH]-linker-[VL]
[VH-linker-[VL]-linker-[VL]-linker-[VH]
[VH]-linker-[VH]-linker-[VL]-linker-[VL]
[VL]-linker-[VL]-linker [VH]-linker-[VH]
[VL]-linker-[VH]-linker-[VL]-linker-[VH]

Any arbitrary peptide linker can be introduced by genetic engineering, and synthetic linkers (see, for example, those disclosed in Protein Engineering, 9(3), 299-305, 1996) or such can be used as linkers for linking the antibody variable regions. In the present invention, peptide linkers are preferable. The length of the peptide linkers is not particularly limited, and can be suitably selected by those skilled in the art according to the purpose. The length of amino acid residues composing a peptide linker is generally 1 to 100 amino acids, preferably 3 to 50 amino acids, more preferably 5 to 30 amino acids, and particularly preferably 12 to 18 amino acids (for example, 15 amino acids).

Any amino acid sequences composing peptide linkers can be used, as long as they do not inhibit the binding activity of scFv. Examples of the amino acid sequences used in peptide linkers include:

| Ser | |
|---|---|
| Gly-Ser | |
| Gly-Gly-Ser | |
| Ser-Gly-Gly | |
| Gly-Gly-Gly-Ser | (SEQ ID NO: 101) |
| Ser-Gly-Gly-Gly | (SEQ ID NO: 102) |
| Gly-Gly-Gly-Gly-Ser | (SEQ ID NO: 103) |
| Ser-Gly-Gly-Gly-Gly | (SEQ ID NO: 104) |
| Gly-Gly-Gly-Gly-Gly-Ser | (SEQ ID NO: 105) |
| Ser-Gly-Gly-Gly-Gly-Gly | (SEQ ID NO: 106) |
| Gly-Gly-Gly-Gly-Gly-Gly-Ser | (SEQ ID NO: 107) |
| Ser-Gly-Gly-Gly-Gly-Gly-Gly | (SEQ ID NO: 108) |
| (Gly-Gly-Gly-Gly-Ser | (SEQ ID NO: 101))n |
| (Ser-Gly-Gly-Gly-Gly | (SEQ ID NO: 102))n |

in which n is an integer of 1 or larger.

The amino acid sequences of the peptide linkers can be selected appropriately by those skilled in the art according to the purpose. For example, n, which determines the length of the peptide linkers, is generally 1 to 5, preferably 1 to 3, more preferably 1 or 2.

Therefore, a particularly preferred embodiment of sc(Fv)2 in the present invention is, for example, the following sc(Fv)2:
[VH]-peptide linker (15 amino acids)-[VL]-peptide linker (15 amino acids)-[VH]-peptide linker (15 amino acids)-[VL]

Alternatively, synthetic chemical linkers (chemical crosslinking agents) can be used to link the V regions. Crosslinking agents routinely used to crosslink peptide compounds and such can be used in the present invention. For example, the following chemical crosslinking agents are known. These crosslinking agents are commercially available:
N-hydroxy succinimide (NHS);
disuccinimidyl suberate (DSS);
bis(sulfosuccinimidyl) suberate (BS3);
dithiobis(succinimidyl propionate) (DSP);
dithiobis(sulfosuccinimidyl propionate) (DTSSP);
ethylene glycol bis(succinimidyl succinate) (EGS);
ethylene glycol bis(sulfosuccinimidyl succinate) (sulfo-EGS);
disuccinimidyl tartrate (DST);
disulfosuccinimidyl tartrate (sulfo-DST);
bis[2-(succinimidoxycarbonyloxy)ethyl]sulfone (BSOCOES); and
bis[2-(sulfosuccinimidoxycarbonyloxy)ethyl]sulfone (sulfo-BSOCOES).

Usually, three linkers are required to link four antibody variable regions. The multiple linkers to be used may all be of the same type or different types. In the present invention, a preferred minibody is a diabody or an sc(Fv)2. Such minibody can be obtained by treating an antibody with an enzyme, such as papain or pepsin, to generate antibody fragments, or by constructing DNAs that encode these antibody fragments, introducing them into expression vectors, and then expressing them in appropriate host cells (see, for example, Co, M. S. et al., J. Immunol. (1994) 152, 2968-2976; Better, M. and Horwitz, A. H., Methods Enzymol. (1989) 178, 476-496; Pluckthun, A. and Skerra, A., Methods Enzymol. (1989) 178, 497-515; Lamoyi, E., Methods Enzymol. (1986) 121, 652-663; Rousseaux, J. et al., Methods Enzymol. (1986) 121, 663-669; and Bird, R. E. and Walker, B. W., Trends Biotechnol. (1991) 9, 132-137).

The antibodies of the present invention include not only monovalent antibodies but also multivalent antibodies. Multivalent antibodies of the present invention include multivalent antibodies whose antigen binding sites are all the same and multivalent antibodies whose antigen binding sites are partially or completely different.

Antibodies bound to various types of molecules such as polyethylene glycol (PEG) can also be used as modified antibodies. Moreover, chemotherapeutic agents, toxic peptides, or cytotoxic substances such as radioactive chemical substances can be bound to the antibodies. Such modified antibodies (hereinafter referred to as antibody conjugates) can be obtained by subjecting the obtained antibodies to chemical modification. Methods for modifying antibodies are already established in this field. Furthermore, as described below, such antibodies can also be obtained in the molecular form of a bispecific antibody designed using genetic engineering technologies to recognize not only LGR7 proteins, but also chemotherapeutic agents, toxic peptides, cytotoxic substances such as radioactive chemical substances, or such. These antibodies are included in the “antibodies” of the present invention.

Chemotherapeutic agents that are bound to the anti-LGR7 antibodies to drive the cytotoxic activity include the following:

azaribine, anastrozole, azacytidine, bleomycin, bortezomib, bryostatin-1, busulfan, camptothecin, 10-hydroxycamptothecin, carmustine, celebrex, chlorambucil, cisplatin, irinotecan, carboplatin, cladribine, cyclophosphamide, cytarabine, dacarbazine, docetaxel, dactinomycin, daunomycin glucuronide, daunorubicin, dexamethasone, diethylstilbestrol, doxorubicin, doxorubicin glucuronide, epirubicin, ethinyl estradiol, estramustine, etoposide, etoposide glucuronide, floxuridine, fludarabine, flutamide, fluorouracil, fluoxymesterone, gemcitabine, hydroxyprogesterone caproate, hydroxyurea, idarubicin, ifosfamide, leucovorin, lomustine, mechlorethamine, medroxyprogesterone acetate, megestrol acetate, melphalan, mercaptopurine, methotrexate, mitoxantrone, mithramycin, mitomycin, mitotane, phenylbutyrate, prednisone, procarbazine, paclitaxel, pentostatin, semustine streptozocin, tamoxifen, taxanes, taxol, testosterone propionate, thalidomide, thioguanine, thiotepa, teniposide, topotecan, uracil mustard, vinblastine, vinorelbine, and vincristine.

In the present invention, preferred chemotherapeutic agents are low-molecular-weight chemotherapeutic agents. Low-molecular-weight chemotherapeutic agents are unlikely to interfere with antibody function even after binding to antibodies. In the present invention, low-molecular-weight chemotherapeutic agents usually have a molecular weight of 100 to 2000, preferably 200 to 1000. Examples of the chemotherapeutic agents demonstrated herein are all low-molecular-weight chemotherapeutic agents. The chemotherapeutic agents of the present invention include prodrugs that are converted to active chemotherapeutic agents in vivo. Prodrug activation may be enzymatic conversion or non-enzymatic conversion.

Moreover, the antibody can be modified with a toxic peptide. Examples of the toxic peptides include the following: Diphtheria toxin A Chain (Langone J. J., et al., Methods in Enzymology, 93,307-308, 1983); Pseudomonas Exotoxin (Nature Medicine, 2, 350-353, 1996); Ricin A Chain (Fulton R. J., et al., J. Biol. Chem., 261, 5314-5319, 1986; Sivam G, et al., Cancer Res., 47, 3169-3173, 1987; Cumber A. J. et al., J. Immunol. Methods, 135, 15-24, 1990; Wawrzynczak E. J., et al., Cancer Res., 50, 7519-7562, 1990; Gheeite V., et al., J. Immunol. Methods, 142, 223-230, 1991); Deglicosylated Ricin A Chain (Thorpe P. E., et al., Cancer Res., 47, 5924-5931, 1987); Abrin A Chain (Wawrzynczak E. J., et al., Br. J. Cancer, 66, 361-366, 1992; Wawrzynczak E. J., et al., Cancer Res., 50, 7519-7562, 1990; Sivam G, et al., Cancer Res., 47, 3169-3173, 1987; Thorpe P. E., et al., Cancer Res., 47, 5924-5931, 1987); Gelonin (Sivam G, et al., Cancer Res., 47, 3169-3173, 1987; Cumber A. J. et al., J. Immunol. Methods, 135, 15-24, 1990; Wawrzynczak E. J., et al., Cancer Res., 50, 7519-7562, 1990; Bolognesi A., et al., Clin. exp. Immunol., 89, 341-346, 1992); Pokeweed anti-viral protein from seeds (PAP-s) (Bolognesi A., et al., Clin. exp. Immunol., 89, 341-346, 1992); Briodin (Bolognesi A., et al., Clin. exp. Immunol., 89, 341-346, 1992); Saporin (Bolognesi A., et al., Clin. exp. Immunol., 89, 341-346, 1992); Momordin (Cumber A. J., et al., J. Immunol. Methods, 135, 15-24, 1990; Wawrzynczak E. J., et al., Cancer Res., 50, 7519-7562, 1990; Bolognesi A., et al., Clin. exp. Immunol., 89, 341-346, 1992); Momorcochin (Bolognesi A., et al., Clin. exp. Immunol., 89, 341-346, 1992); Dianthin 32 (Bolognesi A., et al., Clin. exp. Immunol., 89, 341-346, 1992); Dianthin 30 (Stirpe F., Barbieri L., FEBS letter 195, 1-8, 1986); Modeccin (Stirpe F., Barbieri L., FEBS letter 195, 1-8,1986); Viscumin (Stirpe F., Barbieri L., FEBS letter 195, 1-8, 1986); Volkesin (Stirpe F., Barbieri L., FEBS letter 195, 1-8, 1986); Dodecandrin (Stirpe F., Barbieri L., FEBS letter 195, 1-8, 1986); Tritin (Stirpe F., Barbieri L., FEBS letter 195, 1-8, 1986); Luffin (Stirpe F., Barbieri L., FEBS letter 195, 1-8, 1986); and Trichokírìn (Casellas P., et al., Eur. J. Biochem. 176, 581-588, 1988; Bolognesi A., et al., Clin. exp. Immunol., 89, 341-346, 1992).

In the present invention, “radioactive chemical substance” refers to a chemical substance containing a radioisotope. There is no particular limitation on the radioisotope. Any radioisotope may be used, and for example ³²P, ¹⁴C, ¹²⁵I, ¹³¹I, ¹⁸⁶R, and ¹⁸⁸Re may be utilized.

In another embodiment, one or two or more of the low-molecular-weight chemotherapeutic agents and toxic peptides can be combined and used for antibody modification. The bonding between an anti-LGR7 antibody and the above-mentioned low-molecular weight chemotherapeutic agent may be covalent bonding or non-covalent bonding. Methods for producing antibodies bound to these chemotherapeutic agents are known.

Furthermore, pharmacologically active proteins or peptide toxins can be bound to antibodies by gene recombination technologies. Specifically, for example, it is possible to construct a recombinant vector by fusing a DNA encoding the above-mentioned toxic peptide with a DNA encoding an anti-LGR7 antibody of the present invention in frame, and inserting this into an expression vector. This vector is introduced into suitable host cells, the obtained transformed cells are cultured, and the incorporated DNA is expressed. Thus, an anti-LGR7 antibody bound to the toxic peptide can be obtained as a fusion protein. When obtaining an antibody as a fusion protein, the pharmacologically active protein or toxin is generally fused at the C terminus of the antibody. A peptide linker can be inserted between the antibody and the pharmacologically active protein or toxin.

Furthermore, the antibody of the present invention may be a bispecific antibody. A bispecific antibody refers to an antibody that carries variable regions that recognize different epitopes within the same antibody molecule. The bispecific antibody may have antigen-binding sites that recognize different epitopes on an LGR7 molecule. Two molecules of such a bispecific antibody can bind to one molecule of LGR7. As a result, stronger cytotoxic action can be expected.

Alternatively, the bispecific antibody may be an antibody in which one antigen-binding site recognizes LGR7, and the other antigen-binding site recognizes a cytotoxic substance. Specifically, cytotoxic substances include chemotherapeutic agents, toxic peptides, and radioactive chemical substances. Such a bispecific antibody binds to LGR7-expressing cells, and at the same time, captures cytotoxic substances. This enables the cytotoxic substances to directly act on LGR7-expressing cells. Therefore, bispecific antibodies that recognize cytotoxic substances specifically injure tumor cells and suppress tumor cell proliferation.

Furthermore, in the present invention, bispecific antibodies that recognize antigens other than LGR7 may be combined. For example, it is possible to combine bispecific antibodies that recognize non-LGR7 antigens that are specifically expressed on the surface of target cancer cells like LGR7.

Methods for producing bispecific antibodies are known. For example, two types of antibodies recognizing different antigens may be linked to prepare a bispecific antibody. The antibodies to be linked may be half molecules each having an H chain or an L chain, or may be quarter molecules consisting of only an H chain. Alternatively, hybrid cells producing a bispecific antibody can be prepared by fusing hybridomas producing different monoclonal antibodies. Bispecific antibodies can also be prepared by genetic engineering technologies.

Known methods can be used to measure the antigen-binding activity of the antibodies (Antibodies A Laboratory Manual. Ed Harlow, David Lane, Cold Spring Harbor Laboratory, 1988). For example, an enzyme linked immunosorbent assay (ELISA), an enzyme immunoassay (EIA), a radioimmunoassay (RIA), or a fluoroimmunoassay can be used.

The antibodies of the present invention may be antibodies with a modified sugar chain. It is known that the cytotoxic activity of an antibody can be increased by modifying its sugar chain. Known antibodies having modified sugar chains include the following:
antibodies with modified glycosylation (for example, WO 99/54342);
antibodies deficient in fucose attached to sugar chains (for example, WO 00/61739, WO 02/31140, WO2006/067913, etc.);
antibodies having a sugar chain with bisecting GlcNAc (for example, WO 02/79255), etc.

When the antibodies of the present invention are used for therapy, they are preferably antibodies having cytotoxic activity.

In the present invention, the cytotoxic activity includes, for example, antibody-dependent cell-mediated cytotoxicity (ADCC) activity and complement-dependent cytotoxicity (CDC) activity. In the present invention, CDC activity refers to complement system-mediated cytotoxic activity. ADCC activity refers to the activity of injuring a target cell when a specific antibody attaches to its cell surface antigen. An Fcγ receptor-carrying cell (immune cell, or such) binds to the Fc portion of the antibody via the Fcγ receptor and the target cell is damaged.

An anti-LGR7 antibody of the present invention can be tested to see whether it has ADCC activity or CDC activity using known methods (for example, Current Protocols in Immunology, Chapter 7. Immunologic studies in humans, Editor, John E. Coligan et al., John Wiley & Sons, Inc., (1993) and the like).

First, specifically, effector cells, complement solution, and target cells are prepared.

(1) Preparation of Effector Cells

Spleen is removed from a CBA/N mouse or the like, and spleen cells are isolated in RPMI1640 medium (manufactured by Invitrogen). After washing in the same medium containing 10% fetal bovine serum (FBS, manufactured by HyClone), the cell concentration is adjusted to 5×10⁶/mL to prepare the effector cells.

(2) Preparation of Complement Solution

Baby Rabbit Complement (manufactured by CEDARLANE) is diluted 10-fold in a culture medium (manufactured by Invitrogen) containing 10% FBS to prepare a complement solution.

(3) Preparation of Target Cells

The target cells can be radioactively labeled by incubating cells expressing the LGR7 protein with 0.2 mCi of sodium chromate-⁵¹Cr (manufactured by GE Healthcare Bio-Sciences) in a DMEM medium containing 10% FBS for one hour at 37° C. For LGR7 protein-expressing cells, one may use transformed cells with a LGR7 gene, ovarian cancer cells, or such. After radioactive labeling, cells are washed three times in RPMI1640 medium with 10% FBS, and the target cells can be prepared by adjusting the cell concentration to 2×10⁵ /mL.

ADCC activity or CDC activity can be measured by the method described below. In the case of ADCC activity measurement, the target cell and anti-LGR7 antibody (50 μL each) are added to a 96-well U-bottom plate (manufactured by Becton Dickinson), and reacted for 15 minutes on ice. Thereafter, 100 μL of effector cells are added and incubated in a carbon dioxide incubator for four hours. The final concentration of the antibody is adjusted to 0 or 10 μg/mL. After culturing, 100 μL of the supernatant is collected, and the radioactivity is measured with a gamma counter (COBRAII AUTO-GAMMA, MODEL D5005, manufactured by Packard Instrument Company). The cytotoxic activity (%) can be calculated using the measured values according to the equation: (A−C)/(B−C)×100, wherein A represents the radioactivity (cpm) in each sample, B represents the radioactivity (cpm) in a sample where 1% NP-40 (manufactured by Nacalai Tesque) has been added, and C represents the radioactivity (cpm) of a sample containing the target cells only.

Meanwhile, in the case of CDC activity measurement, 50 μL of target cell and 50 μL of an anti-LGR7 antibody are added to a 96-well flat-bottomed plate (manufactured by Becton Dickinson), and reacted for 15 minutes on ice. Thereafter, 100 μL of the complement solution is added, and incubated in a carbon dioxide incubator for four hours. The final concentration of the antibody is adjusted to 0 or 3 μg/mL. After incubation, 100 μL of supernatant is collected, and the radioactivity is measured with a gamma counter. The cytotoxic activity can be calculated in the same way as in the ADCC activity determination.

On the other hand, in the case of measuring the cytotoxic activity of an antibody conjugate, 50 μL of target cell and 50 μL of an anti-LGR7 antibody conjugate are added to a 96-well flat-bottomed plate (manufactured by Becton Dickinson), and reacted for 15 minutes on ice. This is then incubated in a carbon dioxide incubator for one to four hours. The final concentration of the antibody is adjusted to 0 or 3 μg/mL. After culturing, 100 μL of supernatant is collected, and the radioactivity is measured with a gamma counter. The cytotoxic activity can be calculated in the same way as in the ADCC activity determination.

Another embodiment of the antibodies used in the present invention is an antibody having internalization activity. In the present invention, “antibody having internalization activity” refers to an antibody that is transported into a cell (into the cytoplasm, vesicles, other organelles, and such) upon binding to LGR7 on the cell surface.

Whether or not an antibody has internalization activity can be confirmed using methods known to those skilled in the art. For example, the internalization activity can be confirmed by the method of contacting an anti-LGR7 antibody bound by a labeled substance with LGR7-expressing cells and determining whether the labeled substance is incorporated into the cells, or the method of contacting an anti-LGR7 antibody bound by a cytotoxic substance with LGR7-expressing cells and determining whether or not cell death is induced in the LGR7-expressing cells. More specifically, whether or not an antibody has internalization activity can be checked, for example, by the method described in the Examples below.

An antibody having internalization activity can be used for a pharmaceutical composition such as an anticancer agent, for example, by binding it with the above-mentioned cytotoxic substance.

Any antibody that recognizes LGR7 can be used as the antibody of the present invention. For example, the antibodies of (1) to (29) described below can be exemplified as preferred antibodies. These antibodies can be, for example, full-length antibodies, low-molecular-weight antibodies, animal antibodies, chimeric antibodies, humanized antibodies, or human antibodies.
(1) an antibody comprising an H chain having the amino acid sequence of SEQ ID NO: 5 as CDR1, the amino acid sequence of SEQ ID NO: 6 as CDR2, and the amino acid sequence of SEQ ID NO: 7 as CDR3 (22DA6 heavy chain);
(2) an antibody comprising an L chain having the amino acid sequence of SEQ ID NO: 10 as CDR1, the amino acid sequence of SEQ ID NO: 11 as CDR2, and the amino acid sequence of SEQ ID NO: 12 as CDR3 (221)A6 light chain);
(3) an antibody comprising the H chain of (1) and the L chain of (2) (22DA6);
(4) an antibody comprising an H chain having the amino acid sequence of SEQ ID NO: 15 as CDR1, the amino acid sequence of SEQ ID NO: 16 as CDR2, and the amino acid sequence of SEQ ID NO: 17 as CDR3 (22DA7 heavy chain);
(5) an antibody comprising an L chain having the amino acid sequence of SEQ ID NO: 20 as CDR1, the amino acid sequence of SEQ ID NO: 21 as CDR2, and the amino acid sequence of SEQ ID NO: 22 as CDR3 (22DA7 light chain);
(6) an antibody comprising the H chain of (4) and the L chain of (5) (22DA7);
(7) an antibody comprising an H chain having the amino acid sequence of SEQ. ID NO: 25 as CDR1, the amino acid sequence of SEQ ID NO: 26 as CDR2, and the amino acid sequence of SEQ ID NO: 27 as CDR3 (22DA17 heavy chain);
(8) an antibody comprising an L chain having the amino acid sequence of SEQ ID NO: 30 as CDR1, the amino acid sequence of SEQ ID NO: 31 as CDR2, and the amino acid sequence of SEQ ID NO: 32 as CDR3 (22DA17 light chain);
(9) an antibody comprising the H chain of (7) and the L chain of (8) (22DA17);
(10) an antibody comprising an H chain having the amino acid sequence of SEQ ID NO: 35 as CDR1, the amino acid sequence of SEQ ID NO: 36 as CDR2, and the amino acid sequence of SEQ ID NC): 37 as CDR3 (22DA22 heavy chain);
(11) an antibody comprising an L chain having the amino acid sequence of SEQ ID NO: 40 as CDR1, the amino acid sequence of SEQ ID NO: 41 as CDR2, and the amino acid sequence of SEQ ID NO: 42 as CDR3 (22DA22 light chain);
(12) an antibody comprising the H chain of (10) and the L chain of (11) (22DA22);
(13) an antibody comprising an H chain having the amino acid sequence of SEQ ID NO: 45 as CDR1, the amino acid sequence of SEQ ID NO: 46 as CDR2, and the amino acid sequence of SEQ ID NO: 47 as CDR3 (22DA23 heavy chain);
(14) an antibody comprising an L chain having the amino acid sequence of SEQ ID NO: 50 as CDR1, the amino acid sequence of SEQ ID NO: 51 as CDR2, and the amino acid sequence of SEQ ID NO: 52 as CDR3 (22DA23 light chain);
(15) an antibody comprising the H chain of (13) and the L chain of (14) (22DA23);
(16) an antibody comprising an H chain having the amino acid sequence of SEQ ID NO: 55 as CDR1, the amino acid sequence of SEQ ID NO: 56 as CDR2, and the amino acid sequence of SEQ ID NO: 57 as CDR3 (22DA24 heavy chain);
(17) an antibody comprising an L chain having the amino acid sequence of SEQ ID NO: 60 as CDR1, the amino acid sequence of SEQ ID NO: 61 as CDR2, and the amino acid sequence of SEQ ID NO: 62 as CDR3 (22DA24 light chain);
(18) an antibody comprising the H chain of (16) and the L chain of (17) (22DA24);
(19) an antibody comprising an H chain having the amino acid sequence of SEQ ID NO: 65 as CDR1, the amino acid sequence of SEQ ID NO: 66 as CDR2, and the amino acid sequence of SEQ ID NO: 67 as CDR3 (22SD7 heavy chain);
(20) an antibody comprising an L chain having the amino acid sequence of SEQ ID NO: 70 as CDR1, the amino acid sequence of SEQ ID NO: 71 as CDR2, and the amino acid sequence of SEQ ID NO: 72 as CDR3 (22SD7 light chain);
(21) an antibody comprising the H chain of (19) and the L chain of (20) (22SD7);
(22) an antibody comprising an H chain having the amino acid sequence of SEQ ID NO: 75 as CDR1, the amino acid sequence of SEQ ID NO: 76 as CDR2, and the amino acid sequence of SEQ ID NO: 77 as CDR3 (22SD11 heavy chain);
(23) an antibody comprising an L chain having the amino acid sequence of SEQ ID NO: 80 as CDR1, the amino acid sequence of SEQ ID NO: 81 as CDR2, and the amino acid sequence of SEQ ID NO: 82 as CDR3 (22SD11 light chain);
(24) an antibody comprising the H chain of (22) and the L chain of (23) (22SD11);
(25) an antibody comprising an H chain having the amino acid sequence of SEQ ID NO: 85 as CDR1, the amino acid sequence of SEQ ID NO: 86 as CDR2, and the amino acid sequence of SEQ ID NO: 87 as CDR3 (22SD48 heavy chain);
(26) an antibody comprising an L chain having the amino acid sequence of SEQ ID NO: 90 as CDR1, the amino acid sequence of SEQ ID NO: 91 as CDR2, and the amino acid sequence of SEQ ID NO: 92 as CDR3 (22SD48 light chain);
(27) an antibody comprising the H chain of (25) and the L chain of (26) (22SD48);
(28) an antibody that has equivalent activity as the antibody of any one of (1) to (27);
(29) an antibody that recognizes the same epitope recognized by the antibody of any one of (1) to (27).

In the present invention, “having equivalent activity as an antibody of the present invention” means having binding activity towards LGR7 and/or cytotoxic activity against LGR7-expressing cells that are equivalent to those of an antibody of the present invention.

A method of introducing mutations into polypeptides is one of the methods well known to those skilled in the art for preparing polypeptides that are functionally equivalent to a certain polypeptide. For example, those skilled in the art can prepare an antibody functionally equivalent to an antibody of the present invention by introducing appropriate mutations into the antibody using site-directed mutagenesis (Hashimoto-Gotoh, T. et al. (1995) Gene 152, 271-275; Zoller, M J, and Smith, M. (1983) Methods Enzymol. 100, 468-500; Kramer, W. et al. (1984) Nucleic Acids Res. 12, 9441-9456; Kramer W, and Fritz H J (1987) Methods. Enzymol. 154, 350-367; Kunkel, T A (1985) Proc. Natl. Acad. Sci. USA. 82, 488-492; Kunkel (1988) Methods Enzymol. 85, 2763-2766) and such. Amino acid mutations may also occur naturally. In this way, the antibodies of the present invention also comprise antibodies comprising amino acid sequences with one or more amino acid mutations in the amino acid sequences of the antibodies of the present invention, and which are functionally equivalent to the antibodies of the present invention.

The number of amino acids that are mutated in such mutants is generally considered to be 50 amino acids or less, preferably 30 amino acids or less, and more preferably 10 amino acids or less (for example, 5 amino acids or less).

It is desirable that the amino acid residues are mutated into amino acids in which the properties of the amino acid side chains are conserved. For example, the following categories have been established depending on the amino acid side chain properties:
hydrophobic amino acids (A, I, L, M, F, P, W, Y, and V);
hydrophilic amino acids (R, D, N, C, E, Q, G, H, K, S, and T);
amino acids having aliphatic side chains (G A, V, L, I, and P);
amino acids having hydroxyl-containing side chains (S, T, and Y);
amino acids having sulfur-containing side chains (C and M);
amino acids having carboxylic acid- and amide-containing side chains (D, N, E, and Q);
amino acids having basic side chains (R, K, and H); and
amino acids having aromatic ring-containing side chains (H, F, Y, and W)
(amino acids are represented by one-letter codes in parentheses).

Polypeptides comprising a modified amino acid sequence, in which one or more amino acid residues in a certain amino acid sequence is deleted, added_{;} and/or substituted with other amino acids, are known to retain their original biological activities (Mark, D. F. et al., Proc. Natl. Acad. Sci. USA (1984) 81, 5662-5666; Zoller, M. J. & Smith, M. Nucleic Acids Research (1982) 10, 6487-6500; Wang, A. et al., Science 224, 1431-1433; Dalbadie-McFarland, G et al., Proc. Natl. Acad. Sci. USA (1982) 79, 6409-6413). That is, it is generally said that, in an amino acid sequence constituting a certain polypeptide, the activity of the polypeptide is highly likely to be maintained when amino acids classified into the same group are mutually substituted. In the present invention, the above-mentioned substitution between amino acids within the same amino acid group is referred to as conservative substitution.

Moreover, the present invention also provides antibodies that bind to the same epitope bound by an anti-LGR7 antibody disclosed in the present invention. More specifically, the present invention relates to antibodies that recognize the same epitope recognized by the 22DA6, 22DA7, 22DA17, 22DA22, 22DA23, 22DA24, 22SD7, 22SD11, or 22SD48 antibody, and to the use of such antibodies. These antibodies can be obtained, for example, by the methods described below.

Whether a test antibody shares an epitope with a certain antibody can be determined by their competition for the same epitope. The competition between antibodies is detected by cross-blocking assay and the like. For example, competitive ELISA assay is a preferred cross-blocking assay.

More specifically, in cross-blocking assay, microtiter plate wells coated with an LGR7 protein are preincubated either in the presence or absence of a candidate competing antibody, and an anti-LGR7 antibody of the present invention is added thereto. The quantity of the anti-LGR7 antibody of the present invention that binds to the LGR7 protein in a well correlates indirectly with the binding ability of the candidate competing antibody (test antibody) that competes for binding to the same epitope. In other words, the higher the affinity of a test antibody to the same epitope, the lower the binding of the anti-LGR7 antibody of the present invention to a well coated with the LGR7 protein, and the higher the binding of the test antibody to a well coated with the LGR7 protein.

The quantity of the antibody bound to the well can be easily measured by labeling the antibody in advance. For example, a biotin-labeled antibody can be measured using an avidin/peroxidase conjugate and a suitable substrate. Cross-blocking assay using an enzyme label such as peroxidase is particularly referred to as competitive ELISA assay. The antibody can be labeled with another labeling substance that can be detected or measured. More specifically, radiolabels, fluorescent labels and the like are known.

Alternatively, competitive FACS assay is also a preferable cross-blocking assay.

Specifically, in competitive ELISA assay, instead of using the LGR7 protein to coat the wells of a microtiter plate, LGR7 protein-expressing cells are used. This is pre-incubated in the presence or absence of a candidate competitive antibody, then a biotin-labeled anti-LGR7 antibody of the present invention is added thereto, and a streptavidin/fluorescein conjugate can be used to detect the competition between antibodies. Cross-blocking assay that uses flow cytometry is particularly referred to as competitive FACS assay. The antibody can be labeled with another fluorescent labeling substance that can be detected or measured.

Alternatively, when a test antibody has a constant region derived from a species other than that of the anti-LGR7 antibody of the present invention, each antibody bound to the well can be measured by a labeled antibody that recognizes its constant region. Alternatively, even when the antibodies are derived from the same species, if their classes are different, each antibody bound to the well can be measured with an antibody that recognizes its class.

If a candidate antibody can block the binding of the anti-LGR7 antibody by at least 20%, preferably at least 30%, and more preferably at least 50% compared to the binding activity obtained in a control test performed in the absence of the candidate competing antibody, then the candidate competing antibody either binds to essentially the same epitope as the anti-LGR7 antibody of the present invention, or competes with the anti-LGR7 antibody of the invention for binding to the same epitope. When determining the epitope, the constant region of an antibody of the present invention can be substituted with the same constant region as the test antibody.

### Pharmaceutical Compositions

From a different standpoint, the present invention provides pharmaceutical compositions comprising an LGR7 protein-binding antibody as an active ingredient. Furthermore, the present invention relates to cell growth inhibitors, in particular anticancer agents, which comprise an LGR7 protein-binding antibody as an active ingredient. Preferably, the cell growth inhibitors and anticancer agents of the present invention are administered to a subject who suffers or may suffer from cancer. Since the level of LGR7 expression is very low in normal cells except in the brain but is enhanced in cancer cells, it is thought that cancer cell-specific cytotoxic effect can be obtained by administration of an anti-LGR7 antibody.

There is no particular limitation on the anti-LGR7 antibodies used in the pharmaceutical compositions (for example, anticancer agents) of the present invention, and they may be any anti-LGR7 antibodies. For example, the anti-LGR7 antibodies described above may be used.

In the present invention, “comprising an LGR7-binding antibody as an active ingredient” means comprising an anti-LGR7 antibody as a primary active ingredient, and there is no limitation on the content ratio of the anti-LGR7 antibody.

When the disease targeted by a pharmaceutical composition of the present invention is cancer, the targeted cancer is not particularly limited; however, ovarian cancer is preferred, and ovarian clear cell adenocarcinoma is particularly preferred. The cancer may be either primary or metastatic lesions.

The pharmaceutical compositions of the present invention can be administered orally or parenterally to a patient. Preferably, the administration is parenteral administration. Specifically, the method of administration is, for example, administration by injection, transnasal administration, transpulmonary administration, or transdermal administration. Examples of administration by injection include systemic and local administrations of a pharmaceutical composition of the present invention by intravenous injection, intramuscular injection, intraperitoneal injection, subcutaneous injection, or such. A suitable administration method may be selected according to the age of the patient and symptoms. The dosage may be selected, for example, within the range of 0.0001 mg to 1000 mg per kg body weight in each administration. Alternatively, for example, the dosage for each patient may be selected within the range of 0.001 to 100,000 mg/body. However, the pharmaceutical composition of the present invention is not limited to these doses.

The pharmaceutical compositions of the present invention can be formulated according to conventional methods (for example, Remington's Pharmaceutical Science, latest edition, Mark Publishing Company, Easton, U.S.A), and may also contain pharmaceutically acceptable carriers and additives. Examples include, but are not limited to, surfactants, excipients, coloring agents, perfumes, preservatives, stabilizers, buffers, suspending agents, isotonization agents, binders, disintegrants, lubricants, fluidity promoting agents, and flavoring agents; and other commonly used carriers can be suitably used. Specific examples of the carriers include light anhydrous silicic acid, lactose, crystalline cellulose, mannitol, starch, carmellose calcium, carmellose sodium, hydroxypropyl cellulose, hydroxypropyl methyl cellulose, polyvinylacetal diethylaminoacetate, polyvinylpyrrolidone, gelatin, medium chain fatty acid triglyceride, polyoxyethylene hardened castor oil 60, saccharose, carboxymethyl cellulose, corn starch, inorganic salt, and such.

The present invention also provides methods for damaging or inhibiting proliferation of LGR7-expressing cells by contacting the LGR7-expressing cells with an antibody that binds to the LGR7 protein.

There is no particular limitation on the antibodies used in the methods of the present invention, but for example, the antibodies described above may be used. The cells bound by the anti-LGR7 antibodies are not particularly limited as long as they are cells expressing LGR7. The LGR7-expressing cells in the present invention are preferably cancer cells. More preferably, they are ovarian cancer cells. The methods of the present invention can be applied to both primary and metastatic lesions of these cancers. Even more preferable cancer cells are primary and metastatic ovarian cancer cells.

In the present invention, “contact” is carried out, for example, by adding the antibody to the culture medium of LGR7-expressing cells cultured in vitro. Alternatively, in the present invention, “contact” is carried out by administering to a non-human animal into which cells expressing LGR7 have been transplanted, or to an animal that endogenously has LGR7-expressing cancer cells.

The following method is suitably used as a method for evaluating or measuring cell damage induced in LGR7-expressing cells by contacting them with an anti-LGR7 antibody. Examples of a method for evaluating or measuring the cytotoxic activity in a test tube include methods for measuring the above-mentioned antibody-dependent cell-mediated cytotoxicity (ADCC) activity, complement-dependent cytotoxicity (CDC) activity, and such. Whether or not an anti-LGR7 antibody has ADCC activity or CDC activity can be measured by known methods (for example, Current protocols in Immunology, Chapter 7. Immunologic studies in humans, Editor, John E. Coligan et al., John Wiley & Sons, Inc., (1993) and the like). For activity measurements, an binding antibody having the same isotype as an anti-LGR7 antibody but not having any cytotoxic activity can be used as a control antibody in the same manner as the anti-LGR7 antibody, and it can be determined that the activity is present when the anti-LGR7 antibody shows a stronger cytotoxic activity than the control antibody.

The isotype of an antibody is defined by the sequence of its H chain constant region in the antibody amino acid sequence. The isotype of an antibody is ultimately determined in vivo by class switching that arises from genetic recombinations in chromosomes which occur during maturation of antibody-producing B-cells. Difference in isotype is reflected in the difference of physiological and pathological functions of antibodies. Specifically, for example, the strength of cytotoxic activity is known to be influenced by antibody isotype in addition to the expression level of the antigen. Therefore, when measuring the above-described cell damaging activity, an antibody of the same isotype as the test antibody is preferably used as the control.

To evaluate or measure cell damaging activity in vivo, for example, LGR7-expressing cancer cells are intradermally or subcutaneously transplanted to a non-human test animal, and then a test antibody is intravenously or intraperitoneally administered daily or at the interval of few days, starting from the day of transplantation or the following day. Cytotoxicity can be determined by daily measurement of tumor size. In a similar manner to the evaluation in a test tube, cytotoxicity can be determined by administering a control antibody having the same isotype, and observing that the tumor size in the anti-LGR7 antibody-administered group is significantly smaller than the tumor size in the control antibody-administered group. When using a mouse as the non-human test animal, it is suitable to use a nude (nu/nu) mouse whose thymus has been made genetically defective so that its T lymphocyte function is lost. The use of such a mouse can eliminate the participation of T lymphocytes in the test animals when evaluating or measuring the cytotoxicity of the administered antibody.

Furthermore, the present invention provides cancer diagnostic methods that comprise detecting an LGR7 protein or a gene encoding the LGR7 protein. While LGR7 expression is significantly enhanced in various cancer tissues or cancer cell lines, LGR7 expression in normal cells is very low. Therefore, LGR7 is useful as a marker for specific detection of cancer.

In one embodiment of the methods of the present invention, cancer is diagnosed by detecting the LGR7 protein in a sample. Preferably, an extracellular domain of the LGR7 protein is detected. Detection of the LGR7 protein is preferably carried out using an antibody that recognizes the LGR7 protein.

A specific example of the diagnostic methods of the present invention is a cancer diagnostic method comprising the following steps:
(a) providing a sample collected from a subject; and
(b) detecting an LGR7 protein contained in the collected sample using an LGR7 protein-binding antibody.

In the present invention, “detection” includes both quantitative and qualitative detection. The following measurements are examples of qualitative detection:
measurement to simply determine whether or not the LGR7 protein is present;
measurement to determine whether or not a predetermined amount or more of LGR7 protein is present; and
measurement to compare the quantity of LGR7 protein with that of another sample (for example, a control sample, etc.).

On the other hand, quantitative detection includes measurement of the concentration of LGR7 protein, the quantity of LGR7 protein, and such.

The test sample of the present invention is not particularly limited as long as it is a sample that possibly contains the LGR7 protein. More specifically, a sample collected from the body of an organism such as a mammal is preferable. A sample collected from a human is more preferable. Specific examples of the test sample include blood, interstitial fluid, plasma, extravascular fluid, cerebrospinal fluid, synovial fluid, pleural fluid, serum, lymph, saliva, urine, tissue, ascites, and intraperitoneal lavage. Preferably, the sample is obtained from a test sample such as a specimen of fixed tissues or cells collected from the body of an organism, or liquid culture medium of cells.

The cancer to be diagnosed by the present invention is not particularly limited and can be any kind of cancer. Specific examples include ovarian cancer. In the present invention both primary and metastatic lesions of these cancers can be diagnosed. In the present invention, primary ovarian cancer and metastatic ovarian cancer are particularly preferred cancers.

In the present invention, if the LGR7 protein is detected in a test sample, its level is used as an indicator for cancer diagnosis. Specifically, if the quantity of the LGR7 protein detected in a test sample is greater than that in a negative control or healthy individual, it indicates that the patient has cancer or is highly likely to suffer from cancer in the future. More specifically, the present invention relates to a cancer diagnostic method comprising the following steps:
(1) detecting the level of LGR7 expression in a test sample collected from a subject; and
(2) indicating that the subject has cancer when the level of LGR7 expression detected in (1) is higher than that in a control.

In the present invention, “control” refers to a sample that serves as a standard for comparison, and it includes both a negative control and a biological sample from a healthy individual. A negative control can be obtained by collecting biological samples from healthy individuals and mixing them as necessary. The level of LGR7 expression in the control can be detected in parallel with the level of LGR7 expression in a biological sample from a subject. Alternatively, the level of LGR7 expression in biological samples from a plurality of healthy individuals can be detected in advance, and the standard expression level in healthy individuals can be determined statistically. More specifically, for example, a standard value of mean±2 standard deviations (S.D.) or mean±3 standard deviations (S.D.) can be used. Statistically, the mean±2 standard deviation (S.D.) includes values of 80% of the healthy individuals, and the mean±3 standard deviation (S.D.) includes values of 90% of the healthy individuals.

Alternatively, the level of LGR7 expression in the control can be established using a receiver operating characteristic (ROC) curve. An ROC curve is a graph that shows the detection sensitivity on the vertical axis and the false positive rate (that is, “1-specificity”) on the horizontal axis. In the present invention, an ROC curve can be obtained by plotting changes in the sensitivity and false positive rate when continuously varying the standard value for determining the level of LGR7 expression in a biological sample.

The “standard value” for obtaining an ROC curve is a numerical value temporarily used for statistical analysis. Generally, the “standard value” for obtaining an ROC curve is varied continuously within a range that covers all the standard values that can be selected. For example, the standard value can be varied between the maximum and minimum of the measured values of LGR7 in the population to be analyzed.

Based on the obtained ROC curve, expected standard values for the desired detection sensitivity and accuracy can be selected. A standard value statistically established by an ROC curve and such is also called the “cut-off value”, In cut-off value-based cancer detection methods, in step (2) mentioned above, the LGR7 expression level detected in (1) is compared with the cut-off value. Furthermore, the subject is detected of cancer when the LGR7 expression level detected in (1) is higher than the cut-off value.

In the present invention, the LGR7 expression level can be determined by any method. More specifically, it is possible to determine the LGR7 expression level by evaluating the LGR7 mRNA quantity, LGR7 protein quantity, and biological activity of the LGR7 protein. The quantity of LGR7 mRNA or protein can be determined by the methods described herein.

In the present invention, any animal species expressing an LGR7 protein can be the subject. For example, it is known that many nonhuman mammals such as chimpanzee (Pan troglodytes) (ENSPTRG00000016551), rhesus monkey (Macaca mulatta) (ENSMMUG00000004647), mouse (Mus musculus) (ENSMUSG00000034009), rat (Rattus norvegicus) (ENSRNOG00000024120), guinea pig (Cavia porcellus) (ENSCPOG00000015517), dog (Canis familiaris) (ENSCAFG00000008672), chicken (Gallus gallus) (ENSGALG00000009429) and the like express the LGR7 protein. Thus, these animals are included in the subjects of the present invention. Human is a particularly preferred subject. Needless to say, when a nonhuman animal is used as a subject, the LGR7 protein of the animal species will be detected.

There is no particular limitation on the method for detecting the LGR7 protein contained in a test sample; however, detection by an immunological method exemplified below using the anti-LGR7 antibody is preferable:
radioimmunoassay (RIA);
enzyme immunoassay (EIA);
fluoroimmunoassay (FIA);
luminescence immunoassay (LIA);
immunoprecipitation (IP);
turbidimetric immunoassay (TIA);
Western blotting (WB);
immunohistochemistry (IHC); and
single radial immunodiffusion (SRID).

Of these techniques, the immunohistochemistry (IHC) method comprises the step of detecting the LGR7 protein on a section of fixed tissues or cells collected from a patient suffering from cancer, and it is one of the preferable immunological assays for cancer diagnostic methods. The above immunological methods such as the immunohistochemistry (IHC) method are known to those skilled in the art.

That is, because LGR7 is a membrane protein whose expression is specifically increased in cancer cells, cancer cells or tissues can be detected by an anti-LGR7 antibody. Cancer cells contained in cells or tissues collected from the body are detected by the above-mentioned immunohistological analysis.

In another preferred embodiment, it is possible to detect cancer tissues in vivo using an anti-LGR7 antibody. More specifically, the present invention relates to a method for detecting cancer which comprises the steps of: (1) administering to a subject an LGR7 protein-binding antibody labeled with a labeling substance such as a radioisotope; and (2) detecting accumulation of the labeling substance. To trace the antibody administered into the body, the antibody can be labeled so that it can be detected. For example, it is possible to monitor the in vivo behavior of an antibody labeled with a fluorescent or luminescent substance, or a radioisotope. An antibody labeled with a fluorescent or luminescent substance can be observed using an endoscope or laparoscope. With radioisotope, the localization of the antibody can be imaged by tracing radioactivity. In the present invention, the localization of the anti-LGR7 antibody in vivo represents the presence of cancer cells.

A positron-emitting nuclide can be used as a radioisotope for labeling the antibody for cancer detection in vivo. For example, the antibody can be labeled with a positron-emitting nuclide such as ¹⁸F, ⁵⁵Co, ⁶⁴Cu, ⁶⁶Ga, ⁶⁸Ga, ⁷⁶Br, ⁸⁹Zr and ¹²⁴I. A known method (Acta Oncol. 32, 825-830, 1993) can be used for labeling the anti-LGR7 antibody with such a positron-emitting nuclide.

After administering the anti-LGR7 antibody labeled with a positron-emitting nuclide to a human or animal, the radiation emitted by the radionuclide is measured from outside the body by a positron emission tomography (PET) device, and this is converted to an image using a computerized tomography technique. PET is a device for non-invasive collection of data on the in vivo behavior and such of a drug. The radiation intensity can be quantitatively imaged by PET as signal strength. Using PET as described above, it is possible to detect an antigen molecule that is highly expressed in a specific cancer without collecting a sample from the patient. The anti-LGR7 antibody can be labeled with a short-lived nuclide using a positron-emitting nuclide such as ¹¹C, ¹³N, ¹⁵O, ¹⁸F, ⁴⁵Ti, and such, in addition to the above-mentioned nuclides.

Research and development of the production of short-lived nuclides using the above nuclides by a medical cyclotron, manufacturing techniques for compounds labeled with short-lived nuclides, and the like are progressing. An anti-LGR7 antibody can be labeled with various radioisotopes using these techniques. The anti-LGR7 antibody administered to a patient accumulates in primary and metastatic lesions in accordance with the anti-LGR7 antibody specificity for each site of pathological tissue. If the anti-LGR7 antibody is labeled with a positron-emitting nuclide, by determining radioactivity, the presence of the primary and metastatic lesions can be detected based on the localization of the radioactivity. For the diagnostic applications, the activity value of a gamma-particle or positron emission level of 25 to 4000 keV can be suitably used. In addition, if a suitable nuclide is selected and administered in a large quantity, therapeutic effects can also be expected. To obtain an anticancer effect by radiation, a nuclide providing a gamma-particle or positron emission level of 70 to 700 keV can be used.

In another embodiment of the methods of the present invention, LGR7 gene expression is detected. There is no particular limitation on the gene detected in the present invention; however, mRNA is preferable. In the present invention, “detection” includes both quantitative and qualitative detection. Examples of qualitative detection include the following measuring operations:
measurement to simply determine whether or not LGR7 mRNA is present;
measurement to determine whether or not a predetermined amount or more of LGR7 mRNA is present; and
measurement to comparing the quantity of LGR7 mRNA with that of another sample (for example, a control sample, and such)

On the other hand, quantitative detection includes measurement of the concentration of LGR7 mRNA, the quantity of LGR7 mRNA, and such.

Any sample that possibly contains LGR7 mRNA can be used as a test sample of the present invention. A sample collected from the body of an organism such as a mammal is preferable. A sample collected from a human is more preferable. Specific examples of the test sample include blood, interstitial fluid, plasma, extravascular fluid, cerebrospinal fluid, synovial fluid, pleural fluid, serum, lymph, saliva, urine, tissues, ascites, and intraperitoneal lavage. As a preferable sample, the test sample of the present invention includes a sample obtained from a test sample such as a specimen of fixed tissues or cells collected from the body of an organism, or a culture medium of cells.

When using samples obtained from test samples such as a specimen of fixed tissues or cells collected from the body of an organism, or a culture medium of cells, the in situ hybridization method can be suitably used. The in situ hybridization method has been developed as a means for determining the presence or distribution of specific DNA and RNA molecules in cells and tissues, and their expression intensity. The principle is that the method utilizes the property of specific complex formation of a specified nucleic acid sequence in a cell by a probe nucleic acid having a nucleotide sequence complementary thereto. The in situ hybridization method is used for detection of DNA, RNA, and such in cells, because if the probe is labeled with a radioisotope (RI) or an antigenic substance (hapten) in advance, then the site of hybridization can be distinguished by detecting the label. An RI label can be suitably used as the probe label. A more suitable example is the use of fluorescent labels utilizing non-radioactive substances such as haptens including biotin, digoxigenin, and the like. A particularly suitable example is the use of a detection method by fluorescence in situ hybridization called FISH.

Examples of cancers to be diagnosed include clear cell adenocarcinoma of ovarian cancer. In the present invention, both primary and metastatic lesions of these cancers can be diagnosed.

Any animal species expressing the LGR7 protein can be used as a subject of the present invention. For example, it is known that various mammals other than humans such as mice, rats, rhesus monkeys, and chimpanzees express LGR7. A particularly preferred subject is human. When using a non-human animal species as the subject, LGR7 mRNA of the animal species will be detected.

A specific embodiment of the detection methods is described below. First, a sample is prepared from a subject. Then, LGR7 mRNA contained in the sample is detected. In the present invention, cDNA synthesized from mRNA can also be detected. In the present invention, when an LGR7 mRNA or LGR7-encoding cDNA is detected in the test sample, it is determined that cancer may be possible. For example, when the quantity of LGR7 mRNA or LGR7-encoding cDNA detected in the test sample is greater than that in a negative control or healthy individual, this indicates that the subject has cancer or high possibility of suffering from cancer in the future.

Methods for detecting mRNA are known. More specifically, methods such as northern blotting, RT-PCR, and DNA array can be used in the present invention.

The above detection methods of the present invention can be automated using various automatic detection devices. With automation, it is possible to test a large number of samples in a short time.

The present invention also provides a diagnostic agent or kit for cancer diagnosis which comprises a reagent for detecting the LGR7 protein in a test sample. The diagnostic agent of the present invention contains at least an anti-LGR7 antibody.

The kit for cancer diagnosis can be prepared by combining the cancer diagnostic agent of the present invention with another component used for detecting LGR7. More specifically, the present invention relates to a kit for cancer diagnosis which contains an LGR7-binding antibody and a reagent that detects the binding between the antibody and LGR7. The kit may also include a control sample comprising a biological sample containing LGR7. The kit of the present invention may also be accompanied by instructions explaining the measurement procedure.

All prior art references cited herein are incorporated by reference into this description.

### EXAMPLES

Herein below, the present invention will be specifically described with reference to the Examples, but the technical scope of the present invention is not to be construed as being limited thereto.

### Example 1

### Expression Analysis of Human LGR7 mRNA by Affymetrix U133 Plus 2.0 Array

Total RNA was extracted from surgical specimens of ten ovarian cancer cases that were collected after obtaining written consents at the University of Tokyo Hospital (Japan), and stored by freezing. Herein, the surgical samples were embedded in an OCT compound, and this was sliced and dissolved in TRIZOL (Invitrogen), and then total RNA was extracted according to the method described in the manual attached to the product. At the same time, HE-stained specimens were prepared to confirm that a cancerous part is included. The tissue types of the ten ovarian cancer cases are as follows: clear cell carcinomas (four cases), serous adenocarcinoma (two cases), endometrioid adenocarcinoma (three cases), and carcinosarcoma (one case). Expression analysis was performed by Affymetrix U-133 Plus 2.0 Array using these total RNAs, and genes showing high expression specifically in ovarian clear cell adenocarcinoma were selected. As controls, total RNAs derived from normal tissues (Clontech) and ovarian cancer cell lines (purchased from ATCC, JCRB, and Riken) were used.

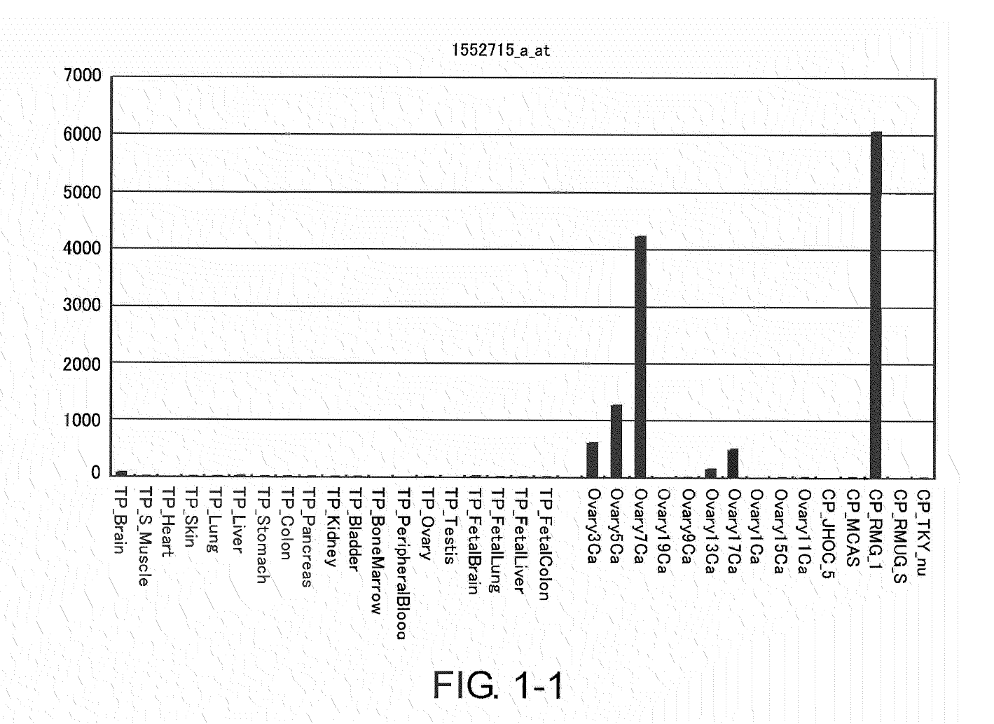

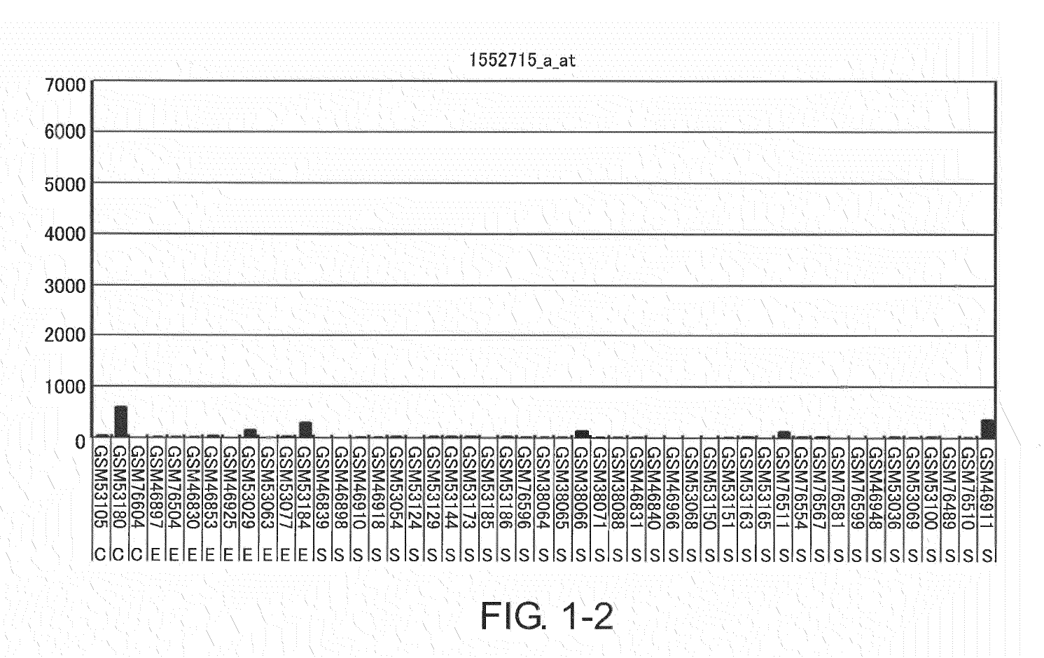

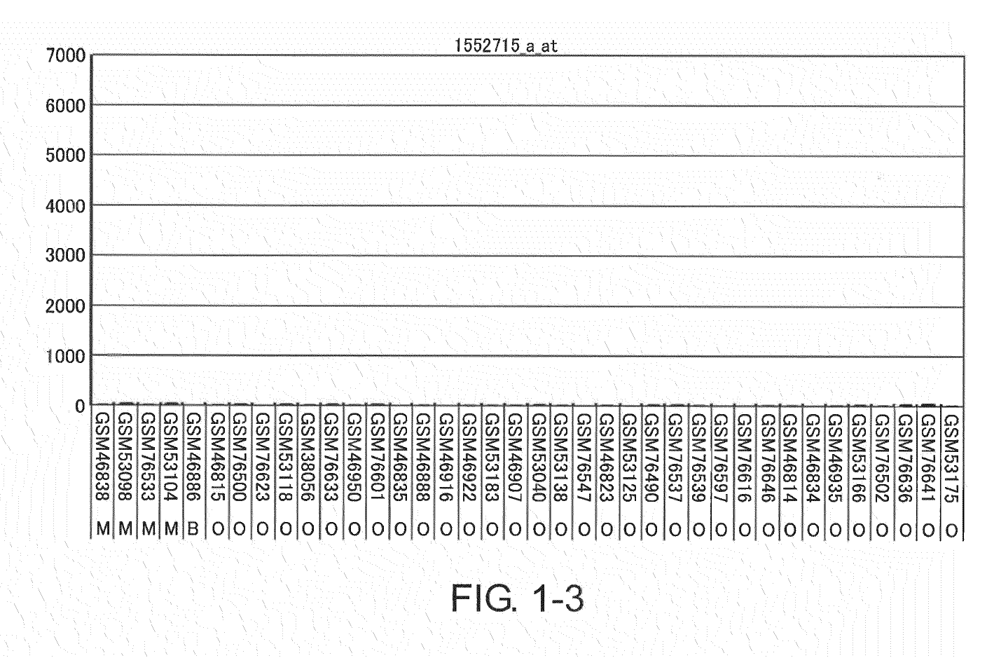

The probe sets were narrowed down to 11761 sets that have an expression level of 200 or more in at least one out of four ovarian clear cell adenocarcinoma cases, which was used as a standard for selecting target molecules suitable for treatment of clear cell adenocarcinoma. Next, by comparing the third highest expression level among the four ovarian clear cell adenocarcinomas cases, and the highest expression level among normal ovary, peripheral blood, bone marrow, and major organs (liver, kidney, lung, stomach, intestine, and pancreas), the probe sets were further narrowed down to 197 sets showing a ratio of 1.8 or more. Of them, LGR7 was selected as the molecule that showed the highest value of the above ratio and which had not been reported to be related to ovarian clear cell adenocarcinoma. When making this selection, expression data of 87 ovarian cancer cases including three clear cell adenocarcinoma cases disclosed by the International Genomics Consortium (IGC) were taken into consideration. FIG. 1 shows a graph indicating the signal levels of LGR7 probe 1552715_a_at in ovarian cancers, ovarian cancer cell lines, and normal tissues. The meaning of the sample names are shown in Table 1.

Among ovarian cancers, LGR7 is expressed specifically in clear cell adenocarcinoma, and thus antitumor agents that target human LGR7 are expected to be effective for this type of cancer.

TABLE 1

| | | |
|---|---|---|
| Brain | normal tissue | brain |
| Muscle | normal tissue | skeletal muscle |
| Heart | normal tissue | heart |
| Skin | normal tissue | skin |
| Lung | normal tissue | lung |
| Liver | normal tissue | liver |
| Stomach | normal tissue | stomach |
| Colon | normal tissue | colon |
| Pancreas | normal tissue | pancreas |
| Kidney | normal tissue | kidney |
| Bone Marrow | normal tissue | bone marrow |
| Peripheral Blood | normal tissue | peripheral blood |
| Ovary | normal tissue | ovary |
| Testis | normal tissue | testis |
| Fetal Brain | normal tissue | fetal brain |
| Fetal Lung | normal tissue | fetal lung |
| Fetal Liver | normal tissue | fetal liver |
| Fetal Colon | normal tissue | fetal colon |
| Ovary3Ca | tissue isolated from | ovarian clear cell |
| | ovarian cancer | adenocarcinoma |
| Ovary5Ca | tissue isolated from | ovarian clear cell |
| | ovarian cancer | adenocarcinoma |
| Ovary7Ca | tissue isolated from | ovarian clear cell |
| | ovarian cancer | adenocarcinoma |
| Ovary19Ca | tissue isolated from | ovarian clear cell |
| | ovarian cancer | adenocarcinoma |
| Ovary9Ca | tissue isolated from | ovarian endometrioid |
| | ovarian cancer | adenocarcinoma |
| Ovary13Ca | tissue isolated from | ovarian endometrioid |
| | ovarian cancer | adenocarcinoma |
| Ovary 17Ca | tissue isolated from | ovarian endometrioid |
| | ovarian cancer | adenocarcinoma |
| Ovary 1Ca | tissue isolated from | ovarian serous |
| | ovarian cancer | adenocarcinoma |
| Ovary 15Ca | tissue isolated from | ovarian serous |
| | ovarian cancer | adenocarcinoma |
| Ovary 11Ca | tissue isolated from | ovarian |
| | ovarian cancer | carcinosarcoma |
| CP_JHOC_5 | ovarian cancer | ovarian clear cell |
| | cell line | adenocarcinoma |
| CP_MCAS | ovarian cancer | ovarian mucinous |
| | cell line | adenocarcinoma |
| CP_RMG_1 | ovarian cancer | ovarian clear cell |
| | cell line | adenocarcinoma |
| CP_RMUG_S | ovarian cancer | ovarian mucinous |
| | cell line | adenocarcinoma |
| CP_TKY_nu | ovarian cancer | ovarian undifferentiated |
| | cell line | adenocarcinoma |

### Example 2

### Establishment of Cells Expressing Full-Length Human LGR7

Full-length human LGR7 cDNA was isolated by the PCR method using Human Uterus QUICK-CLONE cDNA (Clontech) based on NCBI Accession Nos. NP_{—}067647 (SEQ ID NO: 1, amino acid sequence) and NM_{—}021634 (SEQ ID NO: 2, nucleotide sequence). This was cloned into pGEM-T Easy (Promega), and an HA tag sequence was added to the N terminus. Then, this was cloned into the pMCN2i vector for expression in mammalian cells.

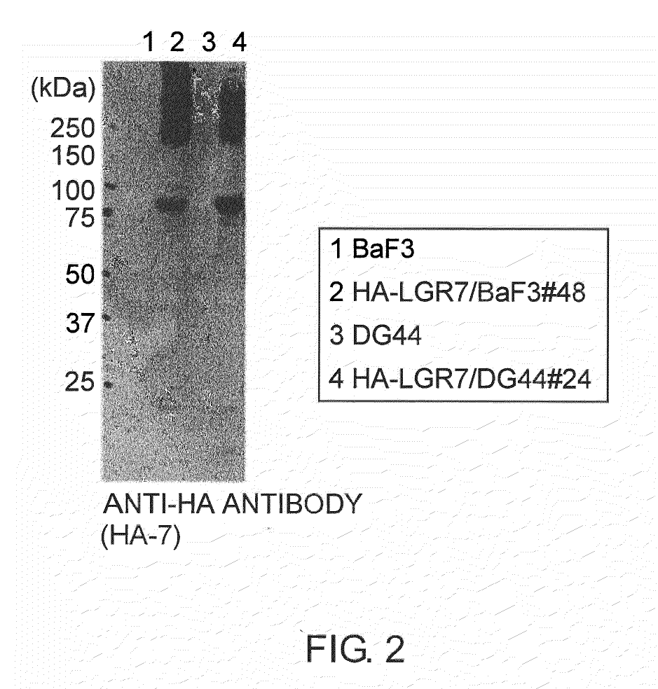

Gene introduction into the Chinese hamster ovary-derived DG44 cell line was carried out using the BioRad GenePulser to obtain the HA-LGR7-expressing cell line HA-LGR7/DG#24. Introduction into Ba/F3 which is a mouse pro-B cell was performed to obtain the HA-LGR7-expressing cell line HA-LGR/BaF3#48. LGR7 expression was confirmed by Western blotting using the HA-7 antibody (Sigma) against the HA tag (FIG. 2).

In addition, a vector into which the LGR7 gene is inserted was constructed for DNA immunization. The expression vector pMCN allows expression of an inserted gene under the mouse CMV promoter (Accession No. U68299), and it has a neomycin resistance gene incorporated as a drug resistance marker. The LGR7 gene was cloned into pMCN using a conventional method to prepare the LGR7 expression vector pMCN-LGR7.

### Example 3

### Preparation of Anti-LGR7 Monoclonal Antibodies by DNA Immunization

DNA immunization by gene introduction into mice was carried out using the GeneGun Particle method. The method was performed according to the manual by Bio-Rad. The bullets for DNA immunization were prepared by mixing 1-mm gold particles (Bio-Rad) and pMCN-LGR7 DNA, and coating the inside of a tube with the mixture. Gene introduction was carried out by shooting the bullets coated with pMCN-LGR7 DNA into the abdominal skin of six-week-old female MRL/1 pr mice using a Helios Gene Gun (Bio-Rad) at a pressure of 200 to 300 psi. It is thought that the gene introduced into keratinocytes, Langerhans cells, and dermal dendritic cells in the skin expresses the LGR7 protein=and thus the cells become antigen-presenting cells (APC) and induce immunity (Methods 31, 232-242 (2003); Immunization with DNA through the skin). DNA immunization was carried out six times at one-week intervals. For the final immunization, 1×10⁶ cells of the BaF3 cell line HA-LGR/BaF3#48 expressing LGR7 were diluted in PBS and then administered into the tail vein. Measurement of the antibody titer was performed by FACS analysis using HA-LGR7/DG#24 cells. The sera from the immunized mice were compared based on their reactivity to the LGR7 protein expressed on the cell membrane surface of HA-LGR7/DG#24 cells. The mouse showing the highest reactivity was subjected to final immunization and cell fusion. The spleen cells were isolated three days after the final immunization, and mixed at 2:1 ratio with P3-X63Ag8U1 mouse myeloma cells (P3U1, purchased from ATCC). Cell fusion was carried out by gradually adding PEG1500 (Roche Diagnostics), and hybridoma cells were prepared. The PEG1500 concentration was diluted by carefully adding RPMI 1640 medium (Gibco BRL), and then PEG1500 was removed by centrifugation. Next, the hybridoma cells were suspended in an RPMI 1640 medium containing 10% FBS, 1× HAT media supplement (SIGMA), and 0.5× BM-Condimed H1 Hybridoma cloning supplement (Roche Diagnostics) (hereinafter, HAT medium), and inoculated into a 96-well culture plate at 200 μL/well. The cell concentration at the time of inoculation was diluted according to the number of P3U1 cells used, and the hybridoma cells were cultured for about one week in HAT medium in the 96-well culture plate at 37° C. under 5% CO₂. Screening of hybridomas that secrete an antibody into the culture supernatant was performed by flow cytometry.

### Example 4

### Preparation of sLGR7Fc

A fragment containing the amino acids of positions 1 to 555 of the LGR7 protein was amplified by PCR, and a vector was constructed to express this fragment as a fusion protein with a human Fc protein (nucleotide sequence, SEQ ID NO: 95; amino acid sequence, SEQ ID NO: 96). The constructed vector was introduced into DG44 cells, and cells that can express the sLGR7Fc fusion protein were selected as a neomycin-resistant strain. The obtained cell strain was subjected to large-scale culture, and the culture supernatant was collected, and the sLGR7Fc protein was purified. The sLGR7Fc protein which was affinity-purified as an Fc fusion protein using an rProtein A column was used as an antigen for protein immunization and screening of hybridomas.

### Example 5

### Production of Anti-LGR7 Antibodies by sLGR7Fc Protein Immunization

50 μg of the affinity-purified sLGR7Fc protein was mixed with Freund's complete adjuvant, and this was subcutaneously immunized into mice. Then, antibodies were induced by subcutaneous immunization of mice twice with a mixture of 50 μg of the sLGR7Fc protein and Freund's incomplete adjuvant. 25 μg of the sLGR7Fc protein was injected into the tail vein of the mouse showing the highest reactivity to the LGR7 protein. After three days, the spleen was removed from the mouse, and subjected to cell fusion with the mouse myeloma cell line P3 X63Ag8U.1, and hybridomas were prepared as in Example 3.

### Example 6

### Evaluation of the Binding Activity by FACS (Flow Cytometry)

The binding of the obtained hybridomas to human LGR7/DG44 cells was evaluated by flow cytometry. The cell line expressing human LGR7 suspended in FACS buffer (2% FBS/PBS/0.05% NaN₃) was diluted to 1×10⁶ cells/mL using FACS buffer, and this was aliquoted into a Falcon 353910 round-bottom 96-well plate at 50 μL/well. The hybridoma culture supernatant diluted to a suitable concentration was added to the wells containing the cells, and this was reacted for 60 minutes on ice. Next, the cells were rinsed once with FACS buffer. As a secondary antibody, a goat F(ab′)₂ fragment anti-mouse IgG Fcγ-FITC (Beckman Coulter) was added to the wells containing the cells, and this was reacted for 30 minutes on ice. After the reaction, the supernatant was removed by centrifugation, and the cells were suspended in 100 μL of FACS buffer, and subjected to flow cytometry. The FACS Calibur (Becton Dickinson) was used for the flow cytometry. A gate for the viable cell population was established using a forward scatter/side scatter dot blot, and an FL1 histogram was produced for the cells included in the population, and their binding activity was evaluated.

The hybridoma supernatants were reacted with DG44 cells induced to express LGR7 and the parental DG44 cells, respectively. Thus, hybridomas that specifically react with LGR7-expressing cells were obtained. The hybridomas from the wells were made into single clones by the limiting dilution method. The isotype of each antibody was analyzed using an IsoStrip® mouse monoclonal antibody isotyping kit (Roche Diagnostics). As a result, the isotype of 22DA6, 22DA7, 22DA11, 22DA12, 22DA23, and 22DA24 is IgG1; and the isotype of 22DA4, 22DA10, 22SD7, 22SD25, 22SD31, and 22SD48 is IgG2a; and the isotype of 22DA17 and 22DA20 is IgG2b. Expansion culture of a single-clone hybridoma was performed, and then the antibody was purified from the culture supernatant using a protein G column according to the manual. The purified antibody was subjected to protein quantification by DC Protein Assay, etc.

### Example 7

### Measurement of the ADCC Activity of the Anti-LGR7 Monoclonal Antibodies

The ADCC activities of the anti-human LGR7 monoclonal antibodies against DG44 cells forcedly expressing LGR7 were evaluated by the chromium release method. Target cells were cultured for a few hours in a culture solution supplemented with Chromium-51 (CHO-S SFM II, manufactured by Invitrogen). Then, the culture solution was removed, and the cells were rinsed with a culture medium. The cells were suspended in a fresh culture medium, and this was added to a 96-well round-bottom plate at 1×10⁴ cells per well. Next, the antibody was added at final concentrations of 1 μg/mL or 0.1 μg/mL, and effector cells (recombinant cells (WO2008/093688) produced by forced expression of a chimeric protein comprising the extracellular domain of mouse Fc-gamma receptor 3 (NM_{—}010188) and the transmembrane and intracellular domains of the human gamma chain (NM_{—}004106) in NK-92 (ATCC, CRL-2407)), were added to each well at approximately five times the amount of the target cells. The plate was left to stand for four hours at 37° C. in a 5% CO₂ incubator. Subsequently, the plate was centrifuged, and a fixed amount of the supernatant was collected from each well. The radioactivity was measured using a Wallac 1480 gamma counter, and the specific chromium release rate (%) was determined using the following equation:

Specific chromium release rate (%)=(A−C)×100/(B−C)

where A represents the radioactivity in each well; B represents the mean value of radioactivity released into the medium upon cell lysis with Nonidet P-40 at a final concentration of 1%; and C represents the mean value of radioactivity when the medium alone is added.

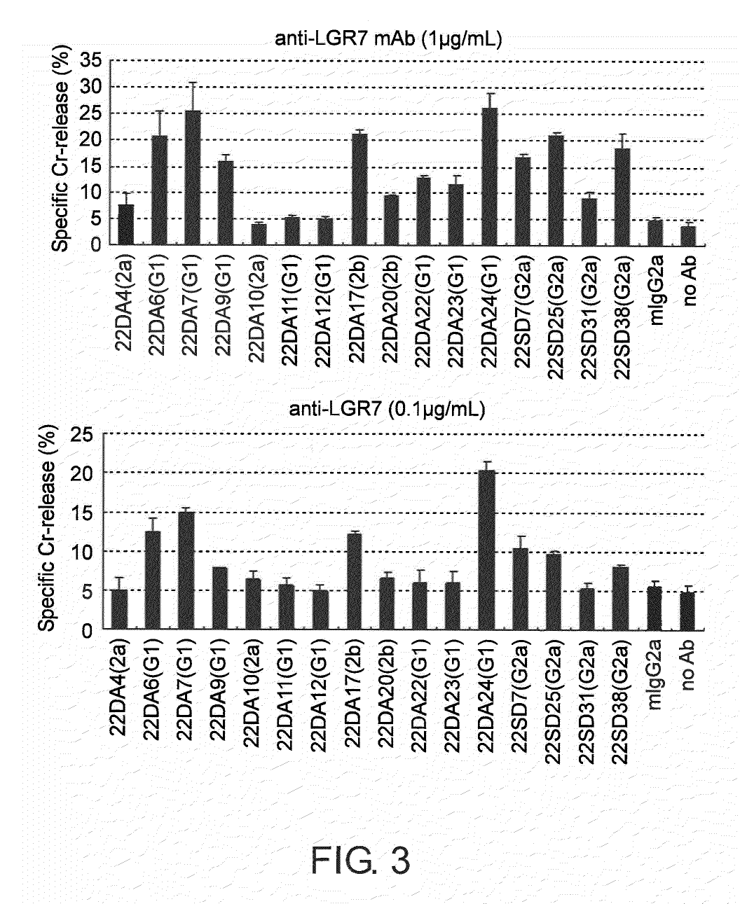

As a result, as indicated in FIG. 3, of the anti-human LGR7 monoclonal antibodies used in the test, in particular 22DA6, 22DA7, 22DA17, 22DA22, 22DA23, 22DA24, 22SD7, 22SD25, and 22SD38 induced very strong ADCC activities against human LGR7-expressing cells. The result indicates that anti-tumor antibody therapy that targets human LGR7 is very useful.

### Example 8

### Measurement of the CDC Activity of the Anti-LGR7 Monoclonal Antibodies

The CDC activity was measured using the extent of 7-AAD uptake by cells in which cell injury has occurred as an indicator.

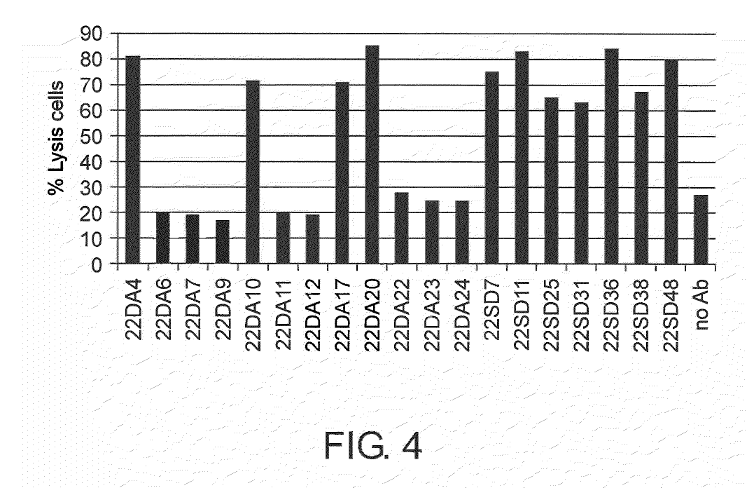

LGR7-expressing DG44 cells were reacted with the monoclonal antibodies at a concentration of 10 μg/mL at 4° C. for 30 minutes. Next, Baby Rabbit Complement (Cedarlane Laboratories) was added thereto at a final concentration of 10%, and the reaction was further performed for 90 minutes at 37° C. After adding 7-AAD (Beckman Coulter) at a final concentration of 1 μg/mL, this was left to stand for 10 minutes at room temperature. Thereafter, the cells were rinsed with FACS buffer, and then the ratio of cells in which cell injury has occurred was evaluated by FACS Calibur. The value of “% FL3” shows the ratio of 7-AAD-stained cells in which cell injury has occurred. As indicated in FIG. 4, multiple anti-LGR7 antibodies showed complement-dependent cytotoxicity (CDC) activity against DG44 cells expressing HA-LGR7.

### Example 9

### Cloning, of the Antigen Genes

Sequences of the antibody variable region genes of the nine hybridomas, 22DA6, 22DA7, 22DA17, 22DA22, 22DA23, 22DA24, 22SD7, 22SD11, and 22SD48, which showed ADCC activity and CDC activity, were determined. The antibody genes were amplified by the RT-PCR method using total RNAs extracted from the hybridomas producing the anti-LGR7 antibodies. Total RNA was extracted from 1×10⁷ hybridoma cells using the RNeasy Plant Mini Kit (QIAGEN). A RACE library was constructed using 1 μg of total RNA and the SMART RACE cDNA Amplification Kit (Clontech). Using synthetic oligonucleotides complementary to the mouse IgG1 constant region sequences, MHC-IgG1 (SEQ ID NO: 97, GGGCCAGTGGATAGACAGATG), MHC-IgG2a (SEQ ID NO: 98, CAGGGGCCAGTGGATAGACCGATG), MHC-IgG2b (SEQ ID NO: 99, CAGGGGCCAGTGGATAGACTGATG), or a synthetic oligonucleotide complementary to the mouse κ chain constant region nucleotide sequence, kappa (SEQ ID NO: 100, GCTCACTGGATGGTGGGAAGATG), a 5′-end gene fragment was amplified from the gene encoding the antibody produced by the hybridoma. Reverse transcription reaction was carried out at 42° C. for 1.5 hours. 50 μL of the PCR solution contained 5 μL of 10× Advantage 2 PCR Buffer, 5 μL of 10×]Universal Primer A Mix, 0.2 mM dNTPs (dATP, dGTP, dCTP, and dTTP), 1 ρL of Advantage 2 Polymerase Mix (all manufactured by Clontech), 2.5 μL of reverse transcription reaction product, and 10 pmol of the synthetic oligonucleotide MHC-IgG1, MHC-IgG2a, MHC-IgG2b, or kappa. PCR reaction was carried out as follows: reaction at a starting temperature of 94° C. for 30 seconds; five cycles of 94° C. for five seconds and 72° C. for three minutes; five cycles of 94° C. for five seconds, 70° C. for ten seconds, and 72° C. for three minutes; and then 25 cycles of 94° C. for five seconds, 68° C. for ten seconds, and 72° C. for three minutes. Finally, the reaction product was heated at 72° C. for seven minutes. Each PCR product was purified from agarose gel using the QIAquick Gel Extraction Kit (manufactured by QIAGEN). Then, the PCR product was cloned into the pGEM-T Easy vector (manufactured by Promega), and its nucleotide sequence was determined.

For 22DA6, the nucleotide and amino acid sequences of the H-chain variable region are shown in SEQ ID NO: 3 and SEQ ID NO: 4, respectively; and the nucleotide and amino acid sequences of the L-chain variable region are shown in SEQ ID NO: 8 and SEQ ID NO: 9, respectively. Furthermore, for 22DA6, SEQ ID NO: 5 shows the amino acid sequence of heavy-chain CDR1, SEQ ID NO: 6 shows the amino acid sequence of heavy-chain CDR2, SEQ ID NO: 7 shows the amino acid sequence of heavy-chain CDR3, SEQ ID NO: 10 shows the amino acid sequence of light-chain CDR1, SEQ ID NO: 11 shows the amino acid sequence of light-chain CDR2, and SEQ ID NO: 12 shows the amino acid sequence of light-chain CDR3.

For 22DA7, the nucleotide and amino acid sequences of the H-chain variable region are shown in SEQ ID NO: 13 and SEQ ID NO: 14, respectively; and the nucleotide and amino acid sequences of the L-chain variable region are shown in SEQ ID NO: 18 and SEQ ID NO: 19, respectively. Furthermore, for 22DA7, SEQ ID NO: 15 shows the amino acid sequence of heavy-chain CDR1, SEQ ID NO: 16 shows the amino acid sequence of heavy-chain CDR2, SEQ ID NO: 17 shows the amino acid sequence of heavy-chain CDR3, SEQ ID NO: 20 shows the amino acid sequence of light-chain CDR1, SEQ ID NO: 21 shows the amino acid sequence of light-chain CDR2, and SEQ ID NO: 22 shows the amino acid sequence of light-chain CDR3.

For 22DA17, the nucleotide and amino acid sequences of the H-chain variable region are shown in SEQ ID NO: 23 and SEQ ID NO: 24, respectively; and the nucleotide and amino acid sequences of the L-chain variable region are shown in SEQ ID NO: 28 and SEQ ID NO: 29, respectively. Furthermore, for 22DA17, SEQ ID NO: 25 shows the amino acid sequence of heavy-chain CDR1, SEQ ID NO: 26 shows the amino acid sequence of heavy-chain CDR2, SEQ ID NO: 27 shows the amino acid sequence of heavy-chain CDR3, SEQ ID NO: 30 shows the amino acid sequence of light-chain CDR1, SEQ ID NO: 31 shows the amino acid sequence of light-chain CDR2, and SEQ ID NO: 32 shows the amino acid sequence of light-chain CDR3.

For 22DA22, the nucleotide and amino acid sequences of the H-chain variable region are shown in SEQ ID NO: 33 and SEQ ID NO: 34, respectively; and the nucleotide and amino acid sequences of the L-chain variable region are shown in SEQ ID NO: 38 and SEQ ID NO: 39, respectively. Furthermore, for 22DA22, SEQ ID NO: 35 shows the amino acid sequence of heavy-chain CDR1, SEQ ID NO: 36 shows the amino acid sequence of heavy-chain CDR2, SEQ ID NO: 37 shows the amino acid sequence of heavy-chain CDR3, SEQ ID NO: 40 shows the amino acid sequence of light-chain CDR1, SEQ ID NO: 41 shows the amino acid sequence of light-chain CDR2, and SEQ ID NO: 42 shows the amino acid sequence of light-chain CDR3.

For 22DA23, the nucleotide and amino acid sequences of the H-chain variable region are shown in SEQ ID NO: 43 and SEQ ID NO: 44, respectively; and the nucleotide and amino acid sequences of the L-chain variable region are shown in SEQ ID NO: 48 and SEQ ID NO: 49, respectively. Furthermore, for 22DA23, SEQ ID NO: 45 shows the amino acid sequence of heavy-chain CDR1, SEQ ID NO: 46 shows the amino acid sequence of heavy-chain CDR2, SEQ ID NO: 47 shows the amino acid sequence of heavy-chain CDR3, SEQ ID NO: 50 shows the amino acid sequence of light-chain CDR1, SEQ ID NO: 51 shows the amino acid sequence of light-chain CDR2, and SEQ ID NO: 52 shows the amino acid sequence of light-chain CDR3.

For 22DA24, the nucleotide and amino acid sequences of the H-chain variable region are shown in SEQ ID NO: 53 and SEQ ID NO: 54, respectively; and the nucleotide and amino acid sequences of the L-chain variable region are shown in SEQ ID NO: 58 and SEQ ID NO: 59, respectively. Furthermore, for 22DA24, SEQ ID NO: 55 shows the amino acid sequence of heavy-chain CDR1, SEQ ID NO: 56 shows the amino acid sequence of heavy-chain CDR2, SEQ ID NO: 57 shows the amino acid sequence of heavy-chain CDR3, SEQ ID NO: 60 shows the amino acid sequence of light-chain CDR1, SEQ ID NO: 61 shows the amino acid sequence of light-chain CDR2, and SEQ ID NO: 62 shows the amino acid sequence of light-chain CDR3.

For 22SD7, the nucleotide and amino acid sequences of the H-chain variable region are shown in SEQ ID NO: 63 and SEQ ID NO: 64, respectively; and the nucleotide and amino acid sequences of the L-chain variable region are shown in SEQ ID NO: 68 and SEQ ID NO: 69, respectively. Furthermore, for 22SD7, SEQ ID NO: 65 shows the amino acid sequence of heavy-chain CDR1, SEQ ID NO: 66 shows the amino acid sequence of heavy-chain CDR2, SEQ ID NO: 67 shows the amino acid sequence of heavy-chain CDR3, SEQ ID NO: 70 shows the amino acid sequence of light-chain CDR1, SEQ ID NO: 71 shows the amino acid sequence of light-chain CDR2, and SEQ ID NO: 72 shows the amino acid sequence of light-chain CDR3.

For 22SD11, the nucleotide and amino acid sequences of the H-chain variable region are shown in SEQ ID NO: 73 and SEQ ID NO: 74, respectively; and the nucleotide and amino acid sequences of the L-chain variable region are shown in SEQ ID NO: 78 and SEQ ID NO: 79, respectively. Furthermore, for 22SD11, SEQ ID NO: 75 shows the amino acid sequence of heavy-chain CDR1, SEQ ID NO: 76 shows the amino acid sequence of heavy-chain CDR2, SEQ ID NO: 77 shows the amino acid sequence of heavy-chain CDR3, SEQ ID NO: 80 shows the amino acid sequence of light-chain CDR1, SEQ ID NO: 81 shows the amino acid sequence of light-chain CDR2, and SEQ ID NO: 82 shows the amino acid sequence of light-chain CDR3.

For 22SD48, the nucleotide and amino acid sequences of the H-chain variable region are shown in SEQ ID NO: 83 and SEQ ID NO: 84, respectively; and the nucleotide and amino acid sequences of the L-chain variable region are shown in SEQ ID NO: 88 and SEQ ID NO: 89, respectively. Furthermore, for 22SD48, SEQ ID NO: 85 shows the amino acid sequence of heavy-chain CDR1, SEQ ID NO: 86 shows the amino acid sequence of heavy-chain CDR2, SEQ ID NO: 87 shows the amino acid sequence of heavy-chain CDR3, SEQ ID NO: 90 shows the amino acid sequence of light-chain CDR1, SEQ ID NO: 91 shows the amino acid sequence of light-chain CDR2, and SEQ ID NO: 92 shows the amino acid sequence of light-chain CDR3.

### Example 10

### Cell-Killing Effect by Internalization of Mab-Zap

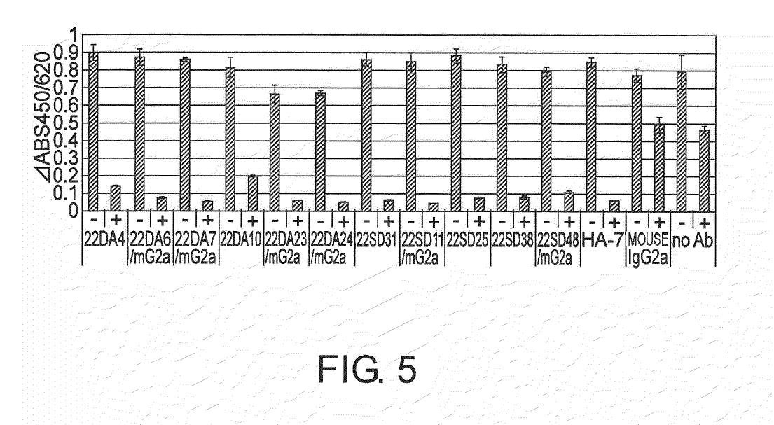

Mab-Zap (manufactured by Advanced Targeting Systems) which is a secondary antibody bound to a toxin called saporin was used to evaluate the cell killing ability against LGR7-expressing cells. For the development model of an antibody pharmaceutical, the mechanism of action is that an antibody bound to a toxin or such binds to a target cell, and is incorporated into the cell, and then kills the target cell by the action of the conjugated toxin. BaF3 cells made to express HA-LGR7 were used. 100 ng of the antibody and 100 ng of Mab-Zap were added to each well. After incubation at 37° C. in a 5% CO₂ incubator for three days, the number of viable cells was determined by WST8 assay using the cell viability assay reagent SF (Nakalai Tesque). The results are shown in FIG. 5. Compared to a control antibody of the same isotype, the values of WST8 assay were clearly lower for the cells co-treated with Mab-ZAP, and the cell-killing effect can be confirmed in all of the analyzed antibodies. This shows that antibodies labeled with a toxin or radioisotope can be incorporated into cells and kill these cells.

### Example 11

### Reactivity of Anti-LGR7 Monoclonal Antibodies to Mouse LGR7

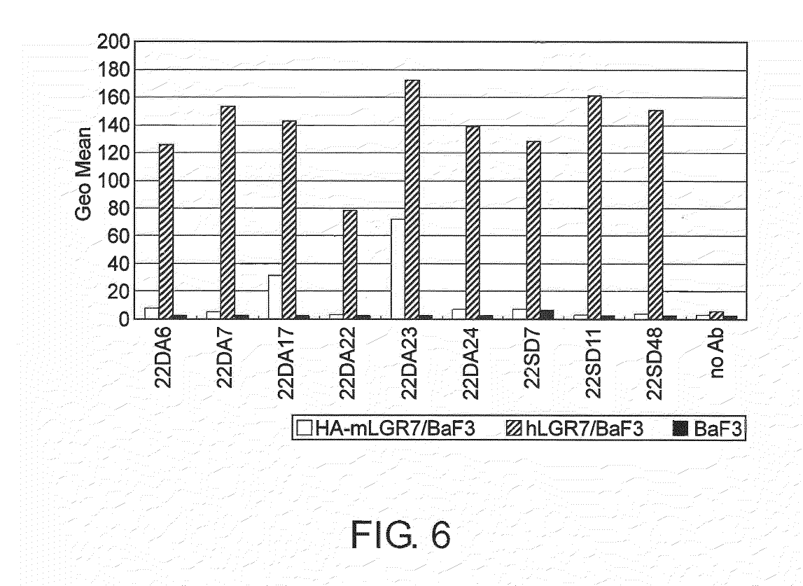

Mouse LGR7 (nucleotide sequence, SEQ ID NO: 93; amino acid sequence, SEQ ID NO: 94) was inserted into an expression vector. Cross-reactivity towards mouse LGR7 was evaluated by flow cytometry using the cell line of forced expression HA-mLGR7/BaF3 obtained by gene introduction into BaF3 cells. As shown in FIG. 6, 22DA17 and 22DA23 were found to exhibit cross-reactivity towards mouse LGR7.

### Example 12

### Epitope Classification by Competition FACS Analysis

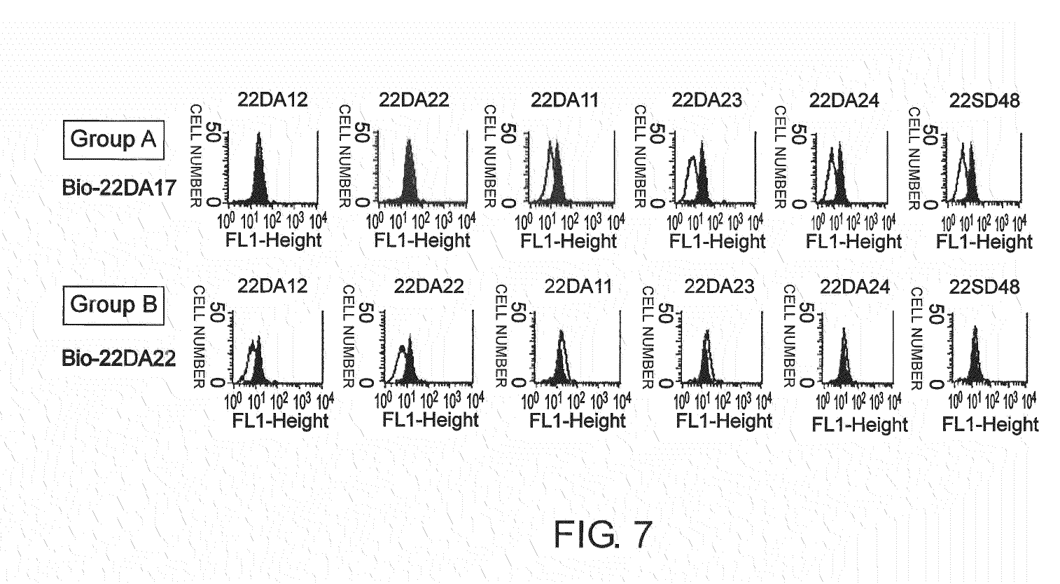

The epitopes were classified by competition FACS analysis. Antibodies were biotinylated using the Biotin Protein Labeling Kit (Roche) according to the manual. Competition FACS analysis is a method in which an excess amount of unlabeled antibody is reacted in advance, a biotinylated antibody is reacted, and the biotinylated antibody is subsequently detected with an FITC-labeled streptavidin. When the antibodies recognize the same epitope, the biotinylated antibody is unable to access the antigen since the unlabeled antibody masks the epitope, and the peak shifts to the left in FACS analysis. When the unlabeled antibody and biotinylated antibody of the same antibody are reacted in order, competition occurs and the shift moves to the left as compared to when an FITC-labeled anti-mouse antibody is reacted. On the other hand, when the antibodies recognize different epitopes, the binding can take place without competition with the unlabeled antibody, and thus the shift to the left is small. Several antibodies were biotinylated and competition assay was carried out. Some of the results are shown in FIG. 7. From competition FACS analysis using the biotinylated Bio-22DA17 and Bio-22DA22 antibodies, it was found that 22DA12 and 22DA22 are antibodies that recognize epitopes different from those of other antibodies.

### Example 13

### Production of a Mouse IgG2a Chimeric Antibody with Mouse IgG2a CH and CL

As methods for enhancing the ADCC activity of an antibody, methods of modifying antibody sugar chains are known. WO2006/067913 and such describe the production of antibodies carrying a sugar chain that does not include α-1,6-core fucose by using CHO cells in which the fucose transporter gene is knocked out (CHO_FTKO).

The H-chain and L-chain variable regions of the antibody gene of the anti-human LGR7 monoclonal antibody 22DA23 cloned as described in Example 9 were respectively amplified by PCR. They were respectively ligated to the H-chain C region Cγ2a and L-chain C region Cκ of a mouse antibody, and this was inserted into an expression vector for mammalian cells so that they can be expressed as a mouse IgG2a chimeric molecule. The obtained vector was transfected into the fucose transporter-deficient CHO cell CHO_FTKO to establish a neomycin-resistant strain.

The cells were cultured in RPMI-1640/10% Ultra-low IgG FBS (Invitrogen)/500 μg/mL Geneticin in the presence of penicillin/streptomycin, and the mouse IgG2a chimeric antibody was purified from the culture supernatant using a Protein A column according to the manual. The purified 22DA23-mIgG2a/FTPKO antibody was subjected to a drug efficacy test using a mouse xenograft model.

### Example 14

### Drug Efficacy Test Using a Mouse Xenograft Model

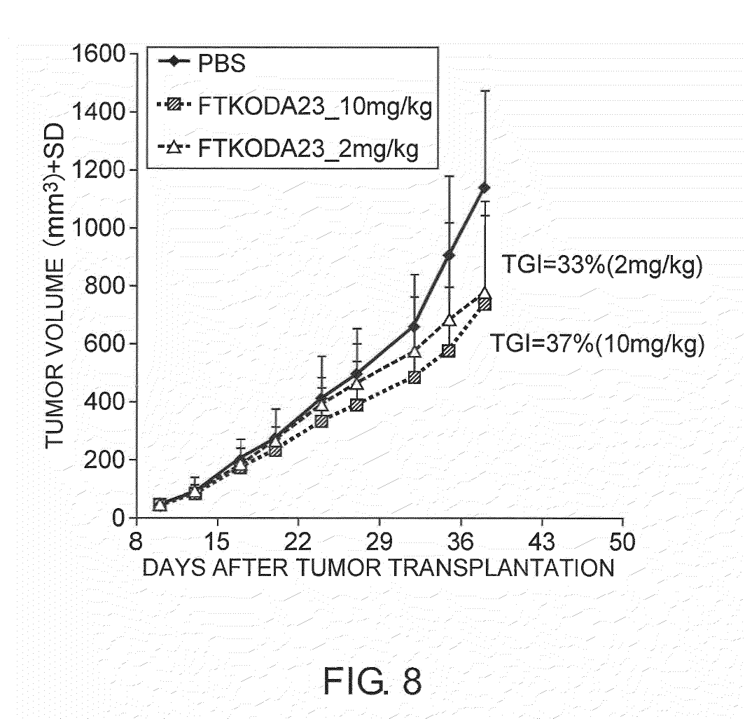

The purified 22DA23-mIgG2a/FTPKO (FTKODA23) antibody was subjected to a drug efficacy test in mice. An approximately 3-mm-square piece of RMG-1 in vivo passaged tumor was transplanted subcutaneously into seven-week-old Scid female mice (Clea Japan). Ten days after transplantation, groups were formed according to the tumor volume and body weight, and they were subjected to the examination. For the RMG-1 in vivo passaged tumor, a tumor isolated 42 days after subcutaneous transplantation of cultured RMG-1 cells at 1e7 cells/body was used. A group included six mice, and the FTKODA23 antibody was administered to the mice once a week at 2 mg/kg or 10 mg/kg. PBS(−) was administered to the control group. The first administration was carried out ten days after transplantation, the second administration 17 days after transplantation, the third administration 24 days after transplantation, and the fourth administration 31 days after transplantation. The tumor volume was measured twice a week, and the final measurement was performed one week after the fourth administration. A graph showing the changes in the tumor volume is presented in FIG. 8. The tumor volumes of the control group and the antibody administration group were compared. TGI (Tumor Growth Inhibition) was 37% on average in the 10 mg/kg administration group and 33% on average in the 2 mg/kg administration group, and the tumor regression effect was confirmed.

### INDUSTRIAL APPLICABILITY

The anti-LGR7 antibodies of the present invention can exhibit anticancer effects by antibody-dependent cell-mediated cytotoxicity activity and complement-dependent cytotoxicity activity towards LGR7-expressing cells. Furthermore, when conjugated to a toxin (cell-damaging substance), they can cause cell injury in LGR7-expressing cells, thus they are useful for diagnosis, prevention, and treatment of various primary and metastatic cancers.

The LGR7 protein-specific antibodies of the present invention are specifically expressed, in particular, in clear cell adenocarcinoma of ovarian cancer, and thus can be used as agents for diagnosing clear cell adenocarcinoma. The diagnostic agents of the present invention are useful for diagnosis of primary and metastatic cancers. More specifically, the possibility of cancer can be evaluated by detecting the LGR7 protein contained in a biological sample collected from a patient. Alternatively, the presence of ovarian clear cell adenocarcinoma can be determined in vivo by detecting the localization of LGR7-expressing cells in vivo.

In addition, the anti-LGR7 antibodies having cytotoxic activity of the present invention are useful for prevention and treatment of cancers expressing the LGR7 protein. More specifically, the present invention provides cytotoxic agents and cell growth inhibitors against cancer cells of ovarian clear cell adenocarcinoma. The cytotoxic agents and cell growth inhibitors against cancer cells of the present invention can be applied to both primary and metastatic cancers.

Furthermore, the anti-LGR7 antibodies having cytotoxic activity of the present invention can be used as therapeutic drugs for ovarian clear cell carcinoma. In the present invention, the anti-LGR7 antibodies are useful as therapeutic drugs against both primary and metastatic cancers.

Additionally, genes encoding the antibodies of the present invention and recombinant cells transformed with the genes can be used to prepare recombinant antibodies that have the above effects or more preferable effects.

1. An antibody that binds to an LGR7 protein and which has cell growth inhibitory activity against cells expressing the LGR7 protein.

2. The antibody of claim 1, wherein the cell growth inhibitory activity is cytotoxic activity.

3. The antibody of claim 2, wherein the cytotoxic activity is antibody-dependent cell-mediated cytotoxicity activity.

4. The antibody of claim 2, wherein the cytotoxic activity is complement-dependent cytotoxicity activity.

5. The antibody of claim 1, which is an antibody to which a cytotoxic substance is bound.

6. The antibody of claim 5, which is an antibody having an internalizing activity.

7. The antibody claim 1, which is an antibody that suppresses the growth of cancer cells.

8. The antibody of claim 7, wherein the cancer cells are clear cell ovarian cancer cells.

9. An antibody of any one of (1) to (29) below:
(1) an antibody comprising an H chain having the amino acid sequence of SEQ ID NO: 5 as CDR1, the amino acid sequence of SEQ ID NO: 6 as CDR2, and the amino acid sequence of SEQ D NO: 7 as CDR3 (22DA6 heavy chain);
(2) an antibody comprising an L chain having the amino acid sequence of SEQ ID NO: 10 as CDR1, the amino acid sequence of SEQ ID NO: 11 as CDR2, and the amino acid sequence of SEQ ID NO: 12 as CDR3 (22DA6 light chain);
(3) an antibody comprising the H chain of (1) and the L chain of (2) (22DA6);
(4) an antibody comprising an H chain having the amino acid sequence of SEQ ID NO: 15 as CDR1, the amino acid sequence of SEQ ID NO: 16 as CDR2, and the amino acid sequence of SEQ ID NO: 17 as CDR3 (22DA7 heavy chain);
(5) an antibody comprising an L chain having the amino acid sequence of SEQ ID NO: 20 as CDR1, the amino acid sequence of SEQ ID NO: 21 as CDR2, and the amino acid sequence of SEQ ID NO: 22 as CDR3 (22DA7 light chain);
(6) an antibody comprising the H chain of (4) and the L chain of (5) (22DA7);
(7) an antibody comprising an H chain having the amino acid sequence of SEQ ID NO: 25 as CDR1, the amino acid sequence of SEQ ID NO: 26 as CDR2, and the amino acid sequence of SEQ ID NO: 27 as CDR3 (22DA17 heavy chain);
(8) an antibody comprising an L chain having the amino acid sequence of SEQ ID NO 30 as CDR1, the amino acid sequence of SEQ ID NO: 31 as CDR2, and the amino acid sequence of SEQ ID NO: 32 as CDR3 (22DA17 light chain);
(9) an antibody comprising the H chain of (7) and the L chain of (8) (22DA17);
(10) an antibody comprising an H chain having the amino acid sequence of SEQ ID NO: 35 as CDR1, the amino acid sequence of SEQ ID NO: 36 as CDR2, and the amino acid sequence of SEQ ID NO: 37 as CDR3 (22DA22 heavy chain);
(11) an antibody comprising an L chain having the amino acid sequence of SEQ ID NO 40 as CDR1, the amino acid sequence of SEQ ID NO: 41 as CDR2, and the amino acid sequence of SEQ ID NO: 42 as CDR3 (22DA22 light chain);
(12) an antibody comprising the H chain of (10) and the L chain of (11) (22DA22);
(13) an antibody comprising an H chain having the amino acid sequence of SEQ ID NO: 45 as CDR1, the amino acid sequence of SEQ ID NO: 46 as CDR2, and the amino acid sequence of SEQ ID NO: 47 as CDR3 (22DA23 heavy chain);
(14) an antibody comprising an L chain having the amino acid sequence of SEQ ID NO: 50 as CDR1, the amino acid sequence of SEQ ID NO: 51 as CDR2, and the amino acid sequence of SEQ ID NO: 52 as CDR3 (22DA23 light chain);
(15) an antibody comprising the H chain of (13) and the L chain of (14) (22DA23);
(16) an antibody comprising an H chain having the amino acid sequence of SEQ ID NO: 55 as CDR1, the amino acid sequence of SEQ ID NO: 56 as CDR2, and the amino acid sequence of SEQ ID NO: 57 as CDR3 (22DA24 heavy chain);
(17) an antibody comprising an L chain having the amino acid sequence of SEQ ID NO: 60 as CDR1, the amino acid sequence of SEQ ID NO: 61 as CDR2, and the amino acid sequence of SEQ ID NO: 62 as CDR3 (22DA24 light chain);
(18) an antibody comprising the H chain of (16) and the L chain of (17) (22DA24);
(19) an antibody comprising an H chain having the amino acid sequence of SEQ ID NO: 65 as CDR1, the amino acid sequence of SEQ ID NO: 66 as CDR2, and the amino acid sequence of SEQ ID NO: 67 as CDR3 (22SD7 heavy chain);
(20) an antibody comprising an L chain having the amino acid sequence of SEQ ID NO: 70 as CDR1, the amino acid sequence of SEQ ID NO: 71 as CDR2, and the amino acid sequence of SEQ ID NO: 72 as CDR3 (22SD7 light chain);
(21) an antibody comprising the H chain of (19) and the L chain of (20) (22SD7);
(22) an antibody comprising an H chain having the amino acid sequence of SEQ ID NO: 75 as CDR1, the amino acid sequence of SEQ ID NO: 76 as CDR2, and the amino acid sequence of SEQ ID NO: 77 as CDR3 (22SD11 heavy chain);
(23) an antibody comprising an L chain having the amino acid sequence of SEQ ID NO: 80 as CDR1, the amino acid sequence of SEQ ID NO: 81 as CDR2, and the amino acid sequence of SEQ ID NO: 82 as CDR3 (22SD11 light chain);
(24) an antibody comprising the H chain of (22) and the L chain of (23) (22SD11);
(25) an antibody comprising an H chain having the amino acid sequence of SEQ ID NO: 85 as CDR1, the amino acid sequence of SEQ ID NO: 86 as CDR2, and the amino acid sequence of SEQ ID NO: 87 as CDR3 (22SD48 heavy chain);
(26) an antibody comprising an L chain having the amino acid sequence of SEQ ID NO: 90 as CDR1, the amino acid sequence of SEQ ID NO: 91 as CDR2, and the amino acid sequence of SEQ ID NO: 92 as CDR3 (22SD48 light chain);
(27) an antibody comprising the H chain of (25) and the L chain of (26) (22SD48);
(28) an antibody that has equivalent activity as the antibody of any one of (1) to (27);
(29) an antibody that recognizes the same epitope recognized by the antibody of any one of (1) to (27).

10. The antibody of claim 1, which has a human constant region.

11. The antibody of claim 10, which is a chimeric antibody, humanized antibody, or human antibody.

12. The antibody of claim 1, which is a fucose-deficient antibody.

13. A pharmaceutical composition comprising the antibody of claim 1 as an active ingredient.

14. A cell growth inhibitor comprising the antibody of claim 1 as an active ingredient.

15. An anticancer agent comprising the antibody of claim 1 as an active ingredient.

16. The anticancer agent of claim 15, wherein the cancer to be treated is ovarian cancer.

17. The anticancer agent of claim 16, wherein the ovarian cancer is clear cell adenocarcinoma.

18. A method for diagnosing cancer, which comprises detecting an LGR7 protein or a gene encoding an LGR7 protein.

19. A method for diagnosing cancer, which comprises detecting an LGR7 protein.

20. The diagnostic method of claim 19, wherein the LGR7 protein is detected using an antibody that binds to the LGR7 protein.

21. A method for diagnosing cancer, comprising:
(a) providing a sample collected from a subject; and
(b) detecting an LGR7 protein contained in the sample of (a) using an antibody that binds to the LGR7 protein.

22. A method for diagnosing cancer, comprising:
(a) administering to a subject an antibody that has binding activity towards an LGR7 protein and which is labeled with a radioisotope; and
(b) detecting accumulation of the radioisotope.

23. The diagnostic method of claim 18, wherein the cancer to be diagnosed is ovarian cancer.

24. The diagnostic method of claim 23, wherein the ovarian cancer is clear cell adenocarcinoma.

25. The antibody of claim 9, which has a human constant region.

26. The antibody of claim 25, which is a chimeric antibody, humanized antibody, or human antibody.

27. The antibody of claim 9, which is a fucose-deficient antibody.

28. A pharmaceutical composition comprising the antibody of claim 9 as an active ingredient.

29. A cell growth inhibitor comprising the antibody of claim 9 as an active ingredient.

30. An anticancer agent comprising the antibody of claim 9 as an active ingredient.

31. The anticancer agent of claim 30, wherein the cancer to be treated is ovarian cancer.

32. The anticancer agent of claim 31, wherein the ovarian cancer is clear cell adenocarcinoma.

33. The diagnostic method of claim 19, wherein the cancer to be diagnosed is ovarian cancer.

34. The diagnostic method of claim 33, wherein the ovarian cancer is clear cell adenocarcinoma.

35. The diagnostic method of claim 21, wherein the cancer to be diagnosed is ovarian cancer.

36. The diagnostic method of claim 35, wherein the ovarian cancer is clear cell adenocarcinoma.

37. The diagnostic method of claim 22, wherein the cancer to be diagnosed is ovarian cancer.

38. The diagnostic method of claim 37, wherein the ovarian cancer is clear cell adenocarcinoma.


SEQUENCE LISTING (108 sequences)

SEQ ID NO: 1 - protein, 757 aa, Homo sapiens
MTSGSVFFYILIFGKYFSHGGGQDVKCSLGYFPCGNITKCLPQLLHCNGVDDCGNQADEDNCGDNNGWSLQFDKYFASYYKMTSQYPFEAETPECLVGSVPVQCLCQGLELDCDETNLRAVPSVSSNVTAMSLQWNLIRKLPPDCFKNYHDLQKLYLQNNKITSISIYAFRGLNSLTKLYLSHNRITFLKPGVFEDLHRLEWLIIEDNHLSRISPPTFYGLNSLILLVLMNNVLTRLPDKPLCQHMPRLHWLDLEGNHIHNLRNLTFISCSNLTVLVMRKNKINHLNENTFAPLQKLDELDLGSNKIENLPPLIFKDLKELSQLNLSYNPIQKIQANQFDYLVKLKSLSLEGIEISNIQQRMFRPLMNLSHIYFKKFQYCGYAPHVRSCKPNTDGISSLENLLASIIQRVFVWVVSAVTCFGNIFVICMRPYIRSENKLYAMSIISLCCADCLMGIYLFVIGGFDLKFRGEYNKHAQLWMESTHCQLVGSLAILSTEVSVLLLTFLTLEKYICIVYPFRCVRPGKCRTITVLILIWITGFIVAFIPLSNKEFFKNYYGTNGVCFPLHSEDTESIGAQIYSVAIFLGINLAAFIIIVFSYGSMFYSVHQSAITATEIRNQVKKEMILAKRFFFIVFTDALCWIPIFVVKFLSLLQVEIPGTITSWVVIFILPINSALNPILYTLTTRPFKEMIHRFWYNYRQRKSMDSKGQKTYAPSFIWVEMWPLQEMPPELMKPDLFTYPCEMSLISQSTRLNSYS

SEQ ID NO: 2 - DNA, 2274 bp, Homo sapiens
atgacatctggttctgtcttcttctacatcttaatttttggaaaatatttttctcatgggggtggacaggatgtcaagtgctcccttggctatttcccctgtgggaacatcacaaagtgcttgcctcagctcctgcactgtaacggtgtggacgactgcgggaatcaggccgatgaggacaactgtggagacaacaatggatggtctctgcaatttgacaaatattttgccagttactacaaaatgacttcccaatatccttttgaggcagaaacacctgaatgtttggtcggttctgtgccagtgcaatgtctttgccaaggtctggagcttgactgtgatgaaaccaatttacgagctgttccatcggtttcttcaaatgtgactgcaatgtcacttcagtggaacttaataagaaagcttcctcctgattgcttcaagaattatcatgatcttcagaagctgtacctgcaaaacaataagattacatccatctccatctatgctttcagaggactgaatagccttactaaactgtatctcagtcataacagaataaccttcctgaagccgggtgtttttgaagatcttcacagactagaatggctgataattgaagataatcacctcagtcgaatttccccaccaacattttatggactaaattctcttattctcttagtcctgatgaataacgtcctcacccgtttacctgataaacctctctgtcaacacatgccaagactacattggctggaccttgaaggcaaccatatccataatttaagaaatttgacttttatttcctgcagtaatttaactgttttagtgatgaggaaaaacaaaattaatcacttaaatgaaaatacttttgcacctctccagaaactggatgaattggatttaggaagtaataagattgaaaatcttccaccgcttatattcaaggacctgaaggagctgtcacaattgaatctttcctataatccaatccagaaaattcaagcaaaccaatttgattatcttgtcaaactcaagtctctcagcctagaagggattgaaatttcaaatatccaacaaaggatgtttagacctcttatgaatctctctcacatatattttaagaaattccagtactgtgggtatgcaccacatgttcgcagctgtaaaccaaacactgatggaatttcatctctagagaatctcttggcaagcattattcagagagtatttgtctgggttgtatctgcagttacctgctttggaaacatttttgtcatttgcatgcgaccttatatcaggtctgagaacaagctgtatgccatgtcaatcatttctctctgctgtgccgactgcttaatgggaatatatttattcgtgatcggaggctttgacctaaagtttcgtggagaatacaataagcatgcgcagctgtggatggagagtactcattgtcagcttgtaggatctttggccattctgtccacagaagtatcagttttactgttaacatttctgacattggaaaaatacatctgcattgtctatccttttagatgtgtgagacctggaaaatgcagaacaattacagttctgattctcatttggattactggttttatagtggctttcattccattgagcaataaggaatttttcaaaaactactatggcaccaatggagtatgcttccctcttcattcagaagatacagaaagtattggagcccagatttattcagtggcaatttttcttggtattaatttggccgcatttatcatcatagttttttcctatggaagcatgttttatagtgttcatcaaagtgccataacagcaactgaaatacggaatcaagttaaaaaagagatgatccttgccaaacgttttttctttatagtatttactgatgcattatgctggatacccatttttgtagtgaaatttctttcactgcttcaggtagaaataccaggtaccataacctcttgggtagtgatttttattctgcccattaacagtgctttgaacccaattctctatactctgaccacaagaccatttaaagaaatgattcatcggttttggtataactacagacaaagaaaatctatggacagcaaaggtcagaaaacatatgctccatcattcatctgggtggaaatgtggccactgcaggagatgccacctgagttaatgaagccggaccttttcacatacccctgtgaaatgtcactgatttctcaatcaacgagactcaattcctattcatga

SEQ ID NO: 3 - DNA, 366 bp, Artificial
  An artificially synthesized nucleotide sequence
caggtccaactgcagcagcctggggctgagctggtgaggcctggagcttcagtgaaactgtcctgcaaggcttctggctactccttcaacagctactggatgaactgggtgaaacagaggcctggacaaggccttgagtggattggcatgtttcatccttccgatagtgaaagtagattaaatcagaagttcaaggacaaggccacattgactgcagacaaatcctccagcacagcctacatgcaactcagcagcccgacatctgaagactctgcggtctattactgtgcaagattggatgatgataactaccccttacaaagtgtggactactggggtcaaggaacctcagtcaccgtctcctca

SEQ ID NO: 4 - protein, 122 aa, Artificial
  An artificially synthesized peptide sequence
QVQLQQPGAELVRPGASVKLSCKASGYSFNSYWMNWVKQRPGQGLEWIGMFHPSDSESRLNQKFKDKATLTADKSSSTAYMQLSSPTSEDSAVYYCARLDDDNYPLQSVDYWGQGTSVTVSS

SEQ ID NO: 5 - protein, 5 aa, Artificial
  An artificially synthesized peptide sequence
SYWMN

SEQ ID NO: 6 - protein, 17 aa, Artificial
  An artificially synthesized peptide sequence
MFHPSDSESRLNQKFKD

SEQ ID NO: 7 - protein, 13 aa, Artificial
  An artificially synthesized peptide sequence
LDDDNYPLQSVDY

SEQ ID NO: 8 - DNA, 321 bp, Artificial
  An artificially synthesized nucleotide sequence
gatatccagatgacacagactacatcctccctgtctgcctctctgggagacagagtcaccatcagttgcagggcaagtcaggacattagcaatcatttaaactggtatcagcagaaaccagatggaactgttaaactcctgatctactacacatcaagattacactcaggagtcccatcaaggttcagtggcagtgggtctggaacagattattcgctcaccattagcaacctggagcaagaagatgttgccacttacttttgccaacagggtaatacgcttccgctcacgttcggtgctgggaccaagctggagctgaaa

SEQ ID NO: 9 - protein, 107 aa, Artificial
  An artificially synthesized peptide sequence
DIQMTQTTSSLSASLGDRVTISCRASQDISNHLNWYQQKPDGTVKLLIYYTSRLHSGVPSRFSGSGSGTDYSLTISNLEQEDVATYFCQQGNTLPLTFGAGTKLELK

SEQ ID NO: 10 - protein, 11 aa, Artificial
  An artificially synthesized peptide sequence
RASQDISNHLN

SEQ ID NO: 11 - protein, 7 aa, Artificial
  An artificially synthesized peptide sequence
YTSRLHS

SEQ ID NO: 12 - protein, 9 aa, Artificial
  An artificially synthesized peptide sequence
QQGNTLPLT

SEQ ID NO: 13 - DNA, 363 bp, Artificial
  An artificially synthesized nucleotide sequence
caggtccaactgcagcagcctggggctgaggttgtgaagcctggggctccagtgaagttgtcctgcaaggcttctggctacaccttcaccacctcctggatgaactgggtgaaacagaggcctggacgaggcctcgagtggattggaaggattgatccttccgatagtgaaactcactacaatcaaaagttcaaggacaaggccaaactgactgtagacaaatcctccagtacagcctacatccaactcagcagcctgacatctgaggactctgcggtctattactgtgcaagagggaactatgattacggcggctatgctgtcgactactggggtcagggaacctcagtcaccgtctcctca

SEQ ID NO: 14 - protein, 121 aa, Artificial
  An artificially synthesized peptide sequence
QVQLQQPGAEVVKPGAPVKLSCKASGYTFTTSWMNWVKQRPGRGLEWIGRIDPSDSETHYNQKFKDKAKLTVDKSSSTAYIQLSSLTSEDSAVYYCARGNYDYGGYAVDYWGQGTSVTVSS

SEQ ID NO: 15 - protein, 5 aa, Artificial
  An artificially synthesized peptide sequence
TSWMN

SEQ ID NO: 16 - protein, 17 aa, Artificial
  An artificially synthesized peptide sequence
RIDPSDSETHYNQKFKD

SEQ ID NO: 17 - protein, 12 aa, Artificial
  An artificially synthesized peptide sequence
GNYDYGGYAVDY

SEQ ID NO: 18 - DNA, 336 bp, Artificial
  An artificially synthesized nucleotide sequence
gatgttgtgatgacccaaagtccactctccctgcctgtcagtcttggagatcaagcctccatctcttgcaaatccagtcagagccttgtacacagtaatggaaacacccatttacattggtacctgaagaggccaggccagtctccaaagctcctgatctacaaagtttccaaccgattttctggggtcccagacaggttcagtggcagtggatcagggacagatttcacactcaagatcagcagagtggaggctgaggatctgggaatttatttctgctctcaaagtacacatgttccgtacacgttcggaggggggaccaagctggaaataaaa

SEQ ID NO: 19 - protein, 112 aa, Artificial
  An artificially synthesized peptide sequence
DVVMTQSPLSLPVSLGDQASISCKSSQSLVHSNGNTHLHWYLKRPGQSPKLLIYKVSNRFSGVPDRFSGSGSGTDFTLKISRVEAEDLGIYFCSQSTHVPYTFGGGTKLEIK

SEQ ID NO: 20 - protein, 16 aa, Artificial
  An artificially synthesized peptide sequence
KSSQSLVHSNGNTHLH

SEQ ID NO: 21 - protein, 7 aa, Artificial
  An artificially synthesized peptide sequence
KVSNRFS

SEQ ID NO: 22 - protein, 9 aa, Artificial
  An artificially synthesized peptide sequence
SQSTHVPYT

SEQ ID NO: 23 - DNA, 354 bp, Artificial
  An artificially synthesized nucleotide sequence
caggtccaactgcagcagtctggggctgagttggtgaggcctggagtttcagtgaagctgtcctgcaaggcttctggctactccctcaccacctactggatgaactgggtgaagcagaggcctggacaaggccttgagtggattggcatgattcatccttccgatagtgaaactgtaataaatcagaagttcaaagacaaggccacattgactgtagacaaatcctccagcacagcctacatgcaactcagcagcccgacatctgaggactctgcggtctattactgtgcaagatgggattacgactactactttgactactggggccaaggcaccactctcacagtctcctca

SEQ ID NO: 24 - protein, 118 aa, Artificial
  An artificially synthesized peptide sequence
QVQLQQSGAELVRPGVSVKLSCKASGYSLTTYWMNWVKQRPGQGLEWIGMIHPSDSETVINQKFKDKATLTVDKSSSTAYMQLSSPTSEDSAVYYCARWDYDYYFDYWGQGTTLTVSS

SEQ ID NO: 25 - protein, 5 aa, Artificial
  An artificially synthesized peptide sequence
TYWMN

SEQ ID NO: 26 - protein, 17 aa, Artificial
  An artificially synthesized peptide sequence
MIHPSDSETVINQKFKD

SEQ ID NO: 27 - protein, 9 aa, Artificial
  An artificially synthesized peptide sequence
WDYDYYFDY

SEQ ID NO: 28 - DNA, 336 bp, Artificial
  An artificially synthesized nucleotide sequence
gatgttttgatgacccaaactccactctccctgcctgtcagtcttggagatcaagcctccatctcttgcagatctagtcagagcatagtacatagtaatggaaacacctatttagaatggtacctgcagaaaccgggccagtctccgaacctcctgatctacaaagtttccaaccgattttctggggtcccagacaggttcagtggcagtggatcagggacagatttcacactcaagatcagcagagtggaggctgaggatctgggagtttattactgctttcaaggttctcatgttccattcacgttcggctcggggacaaagttggaaataaaa

SEQ ID NO: 29 - protein, 112 aa, Artificial
  An artificially synthesized peptide sequence
DVLMTQTPLSLPVSLGDQASISCRSSQSIVHSNGNTYLEWYLQKPGQSPNLLIYKVSNRFSGVPDRFSGSGSGTDFTLKISRVEAEDLGVYYCFQGSHVPFTFGSGTKLEIK

SEQ ID NO: 30 - protein, 16 aa, Artificial
  An artificially synthesized peptide sequence
RSSQSIVHSNGNTYLE

SEQ ID NO: 31 - protein, 7 aa, Artificial
  An artificially synthesized peptide sequence
KVSNRFS

SEQ ID NO: 32 - protein, 9 aa, Artificial
  An artificially synthesized peptide sequence
FQGSHVPFT

SEQ ID NO: 33 - DNA, 363 bp, Artificial
  An artificially synthesized nucleotide sequence
cagattcagcttaaggagtctggacctgttgtcatcaagccatcacagtcactgtctctcacctgcatagtttctggattctccatcacgagtagtcgtttttgctggaattggatccgccagcccccaggaaaggggttagagtggatggggcgcatatgttatgaaggttcaagatactatagtccatccatcaaaagccgcagcaccatctccagagacacatctctgaacaaaatcttttttcagctgaattctgtgacaagcgaggacacagccatgtactactgttccagggaaaaacagattgctgggacggggtttgactactggggccaaggcaccactctcacagtctcctca

SEQ ID NO: 34 - protein, 121 aa, Artificial
  An artificially synthesized peptide sequence
QIQLKESGPVVIKPSQSLSLTCIVSGFSITSSRFCWNWIRQPPGKGLEWMGRICYEGSRYYSPSIKSRSTISRDTSLNKIFFQLNSVTSEDTAMYYCSREKQIAGTGFDYWGQGTTLTVSS

SEQ ID NO: 35 - protein, 7 aa, Artificial
  An artificially synthesized peptide sequence
SSRFCWN

SEQ ID NO: 36 - protein, 16 aa, Artificial
  An artificially synthesized peptide sequence
RICYEGSRYYSPSIKS

SEQ ID NO: 37 - protein, 11 aa, Artificial
  An artificially synthesized peptide sequence
EKQIAGTGFDY

SEQ ID NO: 38 - DNA, 321 bp, Artificial
  An artificially synthesized nucleotide sequence
gacattgtgatgacccagtctcacaaattcatgtccacatcagtaggagacagggtcagtatcacctgcaaggccagtcaggatgtgagtactgctgtagcctggtatcaacagaaaccaggacaatctcctaaactactgatttactcgtcatcctaccgttacactggagtccctgatcgcttcactggcagtggatctgggacggatttcactttcaccatcagcagtgtgcagtctgaagacctggcagtttattactgtcagcaacattatagtactccgctcacgttcggtgctgggaccaagctggagctgaaa

SEQ ID NO: 39 - protein, 107 aa, Artificial
  An artificially synthesized peptide sequence
DIVMTQSHKFMSTSVGDRVSITCKASQDVSTAVAWYQQKPGQSPKLLIYSSSYRYTGVPDRFTGSGSGTDFTFTISSVQSEDLAVYYCQQHYSTPLTFGAGTKLELK

SEQ ID NO: 40 - protein, 11 aa, Artificial
  An artificially synthesized peptide sequence
KASQDVSTAVA

SEQ ID NO: 41 - protein, 7 aa, Artificial
  An artificially synthesized peptide sequence
SSSYRYT

SEQ ID NO: 42 - protein, 9 aa, Artificial
  An artificially synthesized peptide sequence
QQHYSTPLT

SEQ ID NO: 43 - DNA, 357 bp, Artificial
  An artificially synthesized nucleotide sequence
gagatccagctgcagcagtctagacctgagctggtgaggcctggggcttcagtgaaggtatcctgccaggcttctggttatgcattcactaactacaacatgtactgggtgaagcagagccatggaaagagccttgagtggattggatatattgatccttacaatgatgatactacctacaaccagaagttcaagggcaaggccacattgactgttgacaagtcctccagcacagcctacatgcatctcaacagcctgacatctgaggactctgcagtcttttactgtgcaagatttcgactgggatactatgctatggactactggggtcaaggagcctcagtcaccgtctcctca

SEQ ID NO: 44 - protein, 119 aa, Artificial
  An artificially synthesized peptide sequence
EIQLQQSRPELVRPGASVKVSCQASGYAFTNYNMYWVKQSHGKSLEWIGYIDPYNDDTTYNQKFKGKATLTVDKSSSTAYMHLNSLTSEDSAVFYCARFRLGYYAMDYWGQGASVTVSS

SEQ ID NO: 45 - protein, 5 aa, Artificial
  An artificially synthesized peptide sequence
NYNMY

SEQ ID NO: 46 - protein, 17 aa, Artificial
  An artificially synthesized peptide sequence
YIDPYNDDTTYNQKFKG

SEQ ID NO: 47 - protein, 10 aa, Artificial
  An artificially synthesized peptide sequence
FRLGYYAMDY

SEQ ID NO: 48 - DNA, 333 bp, Artificial
  An artificially synthesized nucleotide sequence
gacattgtgctgacccaatctccagcttctttggctgtgtctctagggcagagggccaccatctcctgcaaggccagccaaagtgttgattatgatcttgatagttatatgaactggtaccaacagaaaccaggacagccacccaaactcctcatctatgttgcatccaatctagaatctgggatcccagccaggtttagtggcagtgggtctgggacagacttcaccctcaacatccatcctgtggaggaggaggatgctgcaacctattactgtcaacaaagtaatgaggatccattcacgttcggctcggggacaaagttggaaataaaa

SEQ ID NO: 49 - protein, 111 aa, Artificial
  An artificially synthesized peptide sequence
DIVLTQSPASLAVSLGQRATISCKASQSVDYDLDSYMNWYQQKPGQPPKLLIYVASNLESGIPARFSGSGSGTDFTLNIHPVEEEDAATYYCQQSNEDPFTFGSGTKLEIK

SEQ ID NO: 50 - protein, 15 aa, Artificial
  An artificially synthesized peptide sequence
KASQSVDYDLDSYMN

SEQ ID NO: 51 - protein, 7 aa, Artificial
  An artificially synthesized peptide sequence
VASNLES

SEQ ID NO: 52 - protein, 9 aa, Artificial
  An artificially synthesized peptide sequence
QQSNEDPFT

SEQ ID NO: 53 - DNA, 354 bp, Artificial
  An artificially synthesized nucleotide sequence
caggtccaactgcagcagcctgggcctgagctggtgaggcctggagcttcagtgacgctgtcctgcaaggcttctggctactccttcaccagctactggatgaactgggtgaagcagaggcctggacaaggccttgagtggattggcatgattcatccttccgatagtgaaactatattaaatcagaagttcaaggacaaggccacattgactgtagacaaatcctccagcacagcctccatgcaactcagcagcccgacatctgaggactctgcggtctattactgtgcaagatgcttactaccccactactttgactactggggccaaggcaccactctcacagtctcctca

SEQ ID NO: 54 - protein, 118 aa, Artificial
  An artificially synthesized peptide sequence
QVQLQQPGPELVRPGASVTLSCKASGYSFTSYWMNWVKQRPGQGLEWIGMIHPSDSETILNQKFKDKATLTVDKSSSTASMQLSSPTSEDSAVYYCARCLLPHYFDYWGQGTTLTVSS

SEQ ID NO: 55 - protein, 5 aa, Artificial
  An artificially synthesized peptide sequence
SYWMN

SEQ ID NO: 56 - protein, 17 aa, Artificial
  An artificially synthesized peptide sequence
MIHPSDSETILNQKFKD

SEQ ID NO: 57 - protein, 9 aa, Artificial
  An artificially synthesized peptide sequence
CLLPHYFDY

SEQ ID NO: 58 - DNA, 339 bp, Artificial
  An artificially synthesized nucleotide sequence
gatgttgtgatgacccaagctccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccaaccgattttctggggtcccagacaggttcagtggcagtggatcagggacagatttcacactcaagatcagtagagtggaggctgaggatctgggagtttatttctgctctcaaagtacacatgttcctccattcacgttcggctcggggacaaagttggaaataaaa

SEQ ID NO: 59 - protein, 113 aa, Artificial
  An artificially synthesized peptide sequence
DVVMTQAPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPPFTFGSGTKLEIK

SEQ ID NO: 60 - protein, 16 aa, Artificial
  An artificially synthesized peptide sequence
RSSQSLVHSNGNTYLH

SEQ ID NO: 61 - protein, 7 aa, Artificial
  An artificially synthesized peptide sequence
KVSNRFS

SEQ ID NO: 62 - protein, 10 aa, Artificial
  An artificially synthesized peptide sequence
SQSTHVPPFT

SEQ ID NO: 63 - DNA, 354 bp, Artificial
  An artificially synthesized nucleotide sequence
caggtccaactgcagcagcctggggctgagctggtgaggcctggggcttcagtgaagctgtcctgcaaggcttctggctacacgttcaccagctactggatgaactgggttaagcagaggcctgagcaaggccttgagtggattggaaggattgatccttacgatagtgaaactcactacaatcaaaagttcaaggacaaggccatattgactgtagacaaatcctccagcacagcctacatgcaactcagcagcctgacatctgaggactctgcggtctattactgtgcaagatgggaggtagtagccccctgggactactggggccaaggcaccactctcacagtctcctca

SEQ ID NO: 64 - protein, 118 aa, Artificial
  An artificially synthesized peptide sequence
QVQLQQPGAELVRPGASVKLSCKASGYTFTSYWMNWVKQRPEQGLEWIGRIDPYDSETHYNQKFKDKAILTVDKSSSTAYMQLSSLTSEDSAVYYCARWEVVAPWDYWGQGTTLTVSS

SEQ ID NO: 65 - protein, 5 aa, Artificial
  An artificially synthesized peptide sequence
SYWMN

SEQ ID NO: 66 - protein, 17 aa, Artificial
  An artificially synthesized peptide sequence
RIDPYDSETHYNQKFKD

SEQ ID NO: 67 - protein, 9 aa, Artificial
  An artificially synthesized peptide sequence
WEVVAPWDY

SEQ ID NO: 68 - DNA, 336 bp, Artificial
  An artificially synthesized nucleotide sequence
gatattgtgatgactcaggctgcaccctctgtacctgtcactcctggagagtcagtatccatctcctgcaggtctagtaagagtctcctgcatagtaatggcaacacttacttgtattggttcctgcagaggccaggccagtctcctcagctcctgatatatcggatgtccaaccttgcctcaggagtcccagacaggttcagtggcagtgggtcaggaactgctttcacactgagaatcagtagagtggaggctgaggatgtgggtgtttattactgtatgcaacatctagaatatccgtggacgttcggtggaggcaccaagctggaaatcaaa

SEQ ID NO: 69 - protein, 112 aa, Artificial
  An artificially synthesized peptide sequence
DIVMTQAAPSVPVTPGESVSISCRSSKSLLHSNGNTYLYWFLQRPGQSPQLLIYRMSNLASGVPDRFSGSGSGTAFTLRISRVEAEDVGVYYCMQHLEYPWTFGGGTKLEIK

SEQ ID NO: 70 - protein, 16 aa, Artificial
  An artificially synthesized peptide sequence
RSSKSLLHSNGNTYLY

SEQ ID NO: 71 - protein, 7 aa, Artificial
  An artificially synthesized peptide sequence
RMSNLAS

SEQ ID NO: 72 - protein, 9 aa, Artificial
  An artificially synthesized peptide sequence
MQHLEYPWT

SEQ ID NO: 73 - DNA, 357 bp, Artificial
  An artificially synthesized nucleotide sequence
caggtccaactgcagcagcctggggctgagctggtgaggcctggagcttcagtgaagctgtcctgcaaggcttctggctactccttcaccagctactggatgaactgggtgaagcagaggcctggacaaggccttgagtggattggcatgattcatccttccgatagtgaaactaggttaaatcagaagttcaaggacaaggccacattgactgtagacaaatcctccagcacagcctccatgcaactcagcagcccgacatctgaggactctgcggtctattactgtgcaagagggggggattatccctactactttgactactggggccaaggcaccactctcacagtctcctca

SEQ ID NO: 74 - protein, 119 aa, Artificial
  An artificially synthesized peptide sequence
QVQLQQPGAELVRPGASVKLSCKASGYSFTSYWMNWVKQRPGQGLEWIGMIHPSDSETRLNQKFKDKATLTVDKSSSTASMQLSSPTSEDSAVYYCARGGDYPYYFDYWGQGTTLTVSS

SEQ ID NO: 75 - protein, 5 aa, Artificial
  An artificially synthesized peptide sequence
SYWMN

SEQ ID NO: 76 - protein, 17 aa, Artificial
  An artificially synthesized peptide sequence
MIHPSDSETRLNQKFKD

SEQ ID NO: 77 - protein, 10 aa, Artificial
  An artificially synthesized peptide sequence
GGDYPYYFDY

SEQ ID NO: 78 - DNA, 339 bp, Artificial
  An artificially synthesized nucleotide sequence
gacattgtgatgtcacagtctccatcctccctggctgtgtcagcaggagagaaggtcactatgagctgcaaatccagtcagagtctgctcaacagtagaacccgaaagaactacttggcttggtaccagcagaaaccagggcagtctcctaaactgctgatctactgggcatccactagggaatctggggtccctgatcgcttcacaggcagtggatctgggacagatttcactctcaccatcagcagtgtgcaggctgaagacctggcagtttattactgcaagcactcttataatcttatattcacgttcggctcggggacaaagttggaaataaaa

SEQ ID NO: 79 - protein, 113 aa, Artificial
  An artificially synthesized peptide sequence
DIVMSQSPSSLAVSAGEKVTMSCKSSQSLLNSRTRKNYLAWYQQKPGQSPKLLIYWASTRESGVPDRFTGSGSGTDFTLTISSVQAEDLAVYYCKHSYNLIFTFGSGTKLEIK

SEQ ID NO: 80 - protein, 17 aa, Artificial
  An artificially synthesized peptide sequence
KSSQSLLNSRTRKNYLA

SEQ ID NO: 81 - protein, 7 aa, Artificial
  An artificially synthesized peptide sequence
WASTRES

SEQ ID NO: 82 - protein, 9 aa, Artificial
  An artificially synthesized peptide sequence
KHSYNLIFT

SEQ ID NO: 83 - DNA, 357 bp, Artificial
  An artificially synthesized nucleotide sequence
caggtccaactgcagcagcctggggctgagctggtgaggcctggggcttcagtgaagctgtcctgcaaggcttctggctacacgttcaccagctactgggtgaactgggttaagcagaggcctgaggaaggccttgagtggattggaaggattgatccttacgatagtgaaattcactacaatcaaaagttcaaggacaaggccatattgactgtagacaaatcctccagcacagcctacatgcaactcagcagcctgacatctggggactctgcggtctattactgtgcaaaagggggggaattaccttactactttgactattggggccacggcaccactctcacagtctcctca

SEQ ID NO: 84 - protein, 119 aa, Artificial
  An artificially synthesized peptide sequence
QVQLQQPGAELVRPGASVKLSCKASGYTFTSYWVNWVKQRPEEGLEWIGRIDPYDSEIHYNQKFKDKAILTVDKSSSTAYMQLSSLTSGDSAVYYCAKGGELPYYFDYWGHGTTLTVSS

SEQ ID NO: 85 - protein, 5 aa, Artificial
  An artificially synthesized peptide sequence
SYWVN

SEQ ID NO: 86 - protein, 17 aa, Artificial
  An artificially synthesized peptide sequence
RIDPYDSEIHYNQKFKD

SEQ ID NO: 87 - protein, 10 aa, Artificial
  An artificially synthesized peptide sequence
GGELPYYFDY

SEQ ID NO: 88 - DNA, 336 bp, Artificial
  An artificially synthesized nucleotide sequence
gacattgtgatgtcacagtctccatcctccctggctgtgtcagcaggagagaaggtcactatgagctgcaaatccagtcagaatctgctcaacagtggaacccgaaagaactacttggcttggtaccagcagaaaccagggcagtctcctaaactgctgatctactgggcatccactagggaatctggggtccctgatcgcttcacaggcagtggatctgggacagatttcactctcaccatcagcagtgtgcaggctgaagacctggcagtttattactgcaagcaatcttataatctgtggacgttcggtggaggcaccaagctggaaatcaaa

SEQ ID NO: 89 - protein, 112 aa, Artificial
  An artificially synthesized peptide sequence
DIVMSQSPSSLAVSAGEKVTMSCKSSQNLLNSGTRKNYLAWYQQKPGQSPKLLIYWASTRESGVPDRFTGSGSGTDFTLTISSVQAEDLAVYYCKQSYNLWTFGGGTKLEIK

SEQ ID NO: 90 - protein, 17 aa, Artificial
  An artificially synthesized peptide sequence
KSSQNLLNSGTRKNYLA

SEQ ID NO: 91 - protein, 7 aa, Artificial
  An artificially synthesized peptide sequence
WASTRES

SEQ ID NO: 92 - protein, 8 aa, Artificial
  An artificially synthesized peptide sequence
KQSYNLWT

SEQ ID NO: 93 - protein, 758 aa, Mus musculus
MTSGPFFFCIFIIGKYFTLGSAQDVSCPLGSFPCGNMSRCLPQLLHCNGVDDCGNRADEDHCGDNNGWSLQLDKYFANYYKLASTNSFEAETSECLVGSVPMHCLCRDLELDCDEANLRAVPSVSSNVTVMSLQRNFIRTLPPNGFRKYHELQKLCLQNNRIHSVSVSAFRGLRSLTKLYLSHNRITFLKPGVFEDLHRLEWLIIEDNHLSRISPLTFYGLNSLILLVLMNNALTRLPDKPLCQHMPRLHWLDFEGNRIHNLRNLTFISCNNLTVLVMRKNKINYLNEHAFTHLQKLDELDLGSNKIENLPPNIFKDLKELSQLNISYNPIQKIEVNQFDCLAKLKSLSLEGIEISNIQQRMFRPLINLSHIYFKKFQYCGYAPHVRSCKPNTDGISSLENLLASIIQRVFVWVVSAITCFGNIFVICMRPYIRSENKLHAMSIISLCCADCLMGVYLFVIGAFDLKFRGEYNKHAQPWMESVHCQFMGSLAILSTEVSVLLLTFLTLEKYICIVYPFRCLRPRKCRTITVLIFIWIIGFIVAFAPLGNKEFFKNYYGTNGVCFPLHSEDTGSTGAQIYSVVIFLGINLVAFIIIVFSYGSMFYSVHQSSVTVTEIQKQVKKEVVLAKRFFFIVFTDALCWIPIFILKFLSLLQVEIPDSITSWVVIFILPINSALNPIIYTLTTRPFKEMIHQLWHNYRQRRSVDRKETQKAYAPSFIWVEMWPLQEMSSGFMKPGAFTDPCDLSLVSQSSRLNSYS

SEQ ID NO: 94 - DNA, 2277 bp, Mus musculus
atgacatctggtccattcttcttctgcatcttcattattggaaaatatttcactctcggaagtgcacaggatgtcagttgtcccctcggctccttcccctgtgggaacatgagcaggtgcctgcctcagctgcttcactgcaatggtgtggacgactgcgggaaccgagctgatgaggaccactgcggagacaacaatgggtggtctctgcaactggacaaatattttgctaattactataaactggcttccactaactcctttgaggcagaaacttccgaatgcttggttggctctgtgcccatgcattgtttgtgccgagatctagaacttgactgtgatgaagccaatttacgagctgtcccatcagtttcttccaatgtgactgtaatgtccctgcagaggaacttcataagaacactccctcccaacggcttcaggaagtaccatgagctgcagaagctgtgcctgcaaaacaataggattcactccgtgagcgtctctgccttcagaggactgcgcagcctcactaaactgtacctcagtcataacagaataaccttcctaaagccaggagtttttgaagatctccacagactggaatggctgataattgaagacaatcacctcagtcgaatttccccactcacattttacggactaaattctcttattctcttagtgctgatgaataatgccctcacccgtttgcctgacaaacccctctgtcagcacatgccccgcctgcactggctggactttgaaggcaaccgtatccataatttaagaaatttgacttttatttcctgcaataatttaactgttttggtaatgaggaagaacaaaatcaattacttaaatgaacatgcatttacacacctccagaaactagatgaattggatttaggaagtaataagattgaaaatcttccaccgaacatatttaaggatctgaaggagctgtcgcaattgaatatttcttataacccaatccagaaaattgaagtaaatcaattcgattgtcttgccaaactcaagtctctcagccttgaaggaatagaaatttcaaatatccaacaaaggatgtttagaccgctcataaatctctctcacatctattttaagaaatttcagtactgtggatacgcaccacatgtgcgcagctgtaagcccaacacggatgggatttcatctctagagaacctcttggcaagcatcatccagagagtctttgtctgggttgtatctgccattacctgctttggaaacatttttgttatttgtatgcgaccatacatcagatctgagaacaagctgcatgccatgtcgatcatttctctctgctgtgctgactgcctaatgggggtgtatctatttgtgattggtgcctttgacctcaagtttcggggagagtacaataagcacgcacagccgtggatggagagtgttcattgtcagttcatggggtcgttggccattctgtccacagaagtatccgttttacttttaacatttctgacattggaaaagtacatctgcattgtgtatccttttcggtgtttaaggcctcgaaaatgcagaacaatcacggttctgattttcatttggattattgggtttatagtggcttttgctccacttggcaataaggaatttttcaaaaactactatggcaccaatggagtatgctttcctcttcattcagaagacacgggaagtactggagcccagatttattcagtggtaatttttcttggtattaacctggtggcatttatcatcattgtgttctcctatggaagcatgttttacagtgttcatcaaagctccgtaacagtaaccgaaatacagaagcaagtgaagaaggaggtggttctcgccaaacgctttttctttatcgttttcaccgacgcgctgtgctggattcccatcttcatactgaaatttctttcactgcttcaggtggaaataccagattctattacctcttgggtggtgatttttattctgcctatcaacagtgctttgaacccaattatctacacgttgaccactagacctttcaaggaaatgatccatcaactctggcacaactacagacaaagaaggtctgttgacaggaaagagactcagaaggcatatgctccatcattcatctgggtggaaatgtggcccttgcaggagatgtcctctgggttcatgaagccaggcgctttcacagacccctgtgatctgtcgctagtttctcagtcatctagactcaattcttattcgtaa

SEQ ID NO: 95 - DNA, 691 bp, Homo sapiens
ggtcaccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 96 - protein, 229 aa, Homo sapiens
VTDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 97 - DNA, 21 bp, Artificial
  An artificially synthesized nucleotide sequence
gggccagtggatagacagatg

SEQ ID NO: 98 - DNA, 24 bp, Artificial
  An artificially synthesized nucleotide sequence
caggggccagtggatagaccgatg

SEQ ID NO: 99 - DNA, 24 bp, Artificial
  An artificially synthesized nucleotide sequence
caggggccagtggatagactgatg

SEQ ID NO: 100 - DNA, 23 bp, Artificial
  An artificially synthesized nucleotide sequence
gctcactggatggtgggaagatg

SEQ ID NO: 101 - protein, 4 aa, Artificial
  An artificially synthesized peptide sequence
GGGS

SEQ ID NO: 102 - protein, 4 aa, Artificial
  An artificially synthesized peptide sequence
SGGG

SEQ ID NO: 103 - protein, 5 aa, Artificial
  An artificially synthesized peptide sequence
GGGGS

SEQ ID NO: 104 - protein, 5 aa, Artificial
  An artificially synthesized peptide sequence
SGGGG

SEQ ID NO: 105 - protein, 6 aa, Artificial
  An artificially synthesized peptide sequence
GGGGGS

SEQ ID NO: 106 - protein, 6 aa, Artificial
  An artificially synthesized peptide sequence
SGGGGG

SEQ ID NO: 107 - protein, 7 aa, Artificial
  An artificially synthesized peptide sequence
GGGGGGS

SEQ ID NO: 108 - protein, 7 aa, Artificial
  An artificially synthesized peptide sequence
SGGGGGG